(19) United States
(12) Patent Application Publication
(10) Pub. No.: US 2004/0028613 A1
(43) Pub. Date: Feb. 12, 2004

(54) Dopamine agonist formulations for enhanced central nervous system delivery

(75) Inventor: Steven C. Quay, Edmonds, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Nastech Pharmaceutical Company Inc, Hauppauge, NY

(21) Appl. No.: 09/891,630

(22) Filed: Jun. 25, 2001

(51) Int. Cl. A61K 31/473; A61L 9/04

(52) U.S. Cl. 424/45; 514/295

(57) ABSTRACT

Pharmaceutical formulations are described comprising at least one dopamine receptor agonist and one or more mucosal delivery-enhancing agents for enhanced mucosal delivery of the dopamine receptor agonist. In one aspect, the mucosal delivery formulations and methods provide enhanced delivery of the dopamine receptor agonist to the central nervous sytstem (CNS), for example by yielding dopamine receptor agonist concentrations in the cerebral spinal fluid of 5% or greater of the peak dopamine agonist concentrations in the blood plasma following administration to a mammalian subject. Exemplary formulations and methods within the invention utilize apomorphine as the dopamine receptor agonist. Other exemplary methods and formulations focus in intranasal administration of a dopamine receptor agonist. The formulations and methods of the invention are useful for treating a variety of diseases and conditions in mammalian subjects, including Parkinson's disease, male erectile dysfunction, female sexual dysfunction, among others. In alternate aspects, the mucosal delivery formulations and methods of the invention include one, or any combination of, mucosal delivery-enhancing agents selected from (a) aggregation inhibitory agents; (b) charge modifying agents; (c) pH control agents; (d) degradative enzyme inhibitors; (e) mucolytic or mucus clearing agents; (f) ciliostatic agents; (g) membrane penetration-enhancing agents; (h) modulatory agents of epithelial junction physiology; (i) vasodilator agents; (j) selective transport-enhancing agents; and (k) stabilizing delivery vehicles, carriers, supports or complex-forming agents. These methods and formulations of the invention provide for significantly enhanced absorption of dopamine receptor agonists into or across a nasal mucosal barrier to a target site of action, for example the CNS.

### BACKGROUND OF THE INVENTION

A major disadvantage of drug administration by injection is that trained personnel are often required to administer the drug. For self-administered drugs, many patients are reluctant or unable to give themselves injections on a regular basis. Injection is also associated with increased risks of infection. Other disadvantages of drug injection include variability of delivery results between individuals, as well as unpredictable intensity and duration of drug action.

Despite these noted disadvantages, injection remains the only approved delivery mode for a large assemblage of important therapeutic compounds. These include conventional drugs, as well as a rapidly expanding list of peptide and protein biotherapeutics. Delivery of these compounds via alternate routes of administration, for example, oral, nasal and other mucosal routes, often yields variable results and adverse side effects, and fails to provide suitable bioavailabilty. For macromolecular species in particular, especially peptide and protein therapeutics, alternate routes of administration are limited by susceptibility to inactivation and poor absorption across mucosal barriers.

Mucosal administration of therapeutic compounds may offer certain advantages over injection and other modes of administration, for example in terms of convenience and speed of delivery, as well as by reducing or elimination compliance problems and side effects that attend delivery by injection. However, mucosal delivery is limited of biologically active agents is limited by mucosal barrier functions and other factors. For these reasons, mucosal drug administration typically requires larger amounts of drug than administration by injection. Other therapeutic compounds, including large molecule drugs, peptides and proteins, are often refractory to mucosal delivery.

The ability of drugs to permeate mucosal surfaces, unassisted by delivery-enhancing agents, appears to be related to a number of factors, including molecular size, lipid solubility, and ionization. Small molecules, less than about 300-1,000 daltons, are often capable of penetrating mucosal barriers, however, as molecular size increases, permeability decreases rapidly. Lipid-soluble compounds are generally more permeable through mucosal surfaces than are non-lipid-soluble molecules. Peptides and proteins are poorly lipid soluble, and hence exhibit poor absorption characteristics across mucosal surfaces.

In addition to their poor intrinsic permeability, large macromolecular drugs, including proteins and peptides, are often subject to limited diffusion, as well as lumenal and cellular enzymatic degradation and rapid clearance at mucosal sites. These mucosal sites generally serve as a first line of host defense against pathogens and other adverse environmental agents that come into contact with the mucosal surface. Mucosal tissues provide a substantial barrier to the free diffusion of macromolecules, while enzymatic activities present in mucosal secretions can severely limit the bioavailability of therapeutic agents, particularly peptides and proteins. At certain mucosal sites, such as the nasal mucosa, the typical residence time of proteins and other macromolecular species delivered is limited, e.g., to about 15-30 minutes or less, due to rapid mucociliary clearance.

Various methods and formulations have been attempted to enhance the absorption of drugs across mucosal surfaces. Penetration enhancing substances that facilitate the transport of solutes across biological membranes are widely reported in the art for facilitating mucosal drug delivery (See, e.g., Lee et al., 8 Critical Reviews in Therapeutic Drug Carrier Systems 91, 1991). Mucosal penetration enhancers represented in these reports include (a) chelators (e.g., EDTA, citric acid, salicylates), (b) surfactants (e.g., sodium dodecyl sulfate (SDS)), (c) non-surfactants (e.g., unsaturated cyclic ureas), (d) bile salts (e.g., sodium deoxycholate, sodium taurocholate), and (e) fatty acids (e.g., oleic acid, acylcarnitines, mono- and diglycerides). Numerous additional agents and mechanisms have been proposed for enhancing mucosal penetration of drugs. These include, for example, reducing the viscosity and/or elasticity of mucus layers that cover mucosal surfaces; facilitating transcellular transport by increasing the fluidity of the lipid bilayer of membranes; altering the physicochemical properties (e.g., lipophilicity, stability) of drugs; facilitating paracellular transport by altering tight junctions across the epithelial cell layer; overcoming enzymatic barriers; and increasing the thermodynamic activity of candidate drugs.

While many penetration enhancing methods and additives have been reported to be effective in improving mucosal drug delivery, few penetration enhanced products have been developed and approved for mucosal delivery of drugs. This failure can be attributed to a variety of factors, including poor safety profiles relating to mucosal irritation, and undesirable disruption of mucosal barrier functions.

In view of the foregoing, there remains a substantial unmet need in the art for new methods and tools to facilitate mucosal delivery of biotherapeutic compounds. Related to this need, there is a compelling need in the art for methods and formulations to facilitate mucosal delivery of biotherapeutic compounds that have heretofore proven refractory to delivery via this route, to avail the medical community of the numerous potential advantages of mucosal drug delivery.

One group of therapeutic compounds of interest for mucosal delivery are dopamine receptor agonists, for example apomorphine and its pharmaceutically acceptable salts and derivatives. As reviewed by Hagell and Odin in J. Neurosci. Nurs., 33(1):21-34, 37-8, February 2001, apomorphine is a potent, nonselective, direct-acting dopamine agonist that works by binding to dopamine receptors, primarily in the central nervous system (CNS).

Given subcutaneously, apomporphine has a rapid onset of antiparkinsonian action qualitatively comparable to that of levodopa. Despite its long history, it was not until peripheral dopaminergic side effects could be controlled by oral domperidone that the clinical usefulness of apomorphine in Parkinson's disease began to be investigated thoroughly in the mid-1980s. Although several routes have been tried, subcutaneous administration, either as intermittent injections or continuous infusion, is so far the most common application in the treatment of advanced, fluctuating Parkinson's disease. However, methods to increase the amount of the dose reaching the cerebral spinal fluid (CSF) are needed.

Studies with males show that sublingual administration of dopamine agonists such as apomorphine can be used to induce an erection in a psychogenic male patient, as long as the apomorphine dose required to achieve a significant erectile response is not accompanied by nausea and vomiting or other serious undesirable side effects such as arterial hypotension, flushing and diaphoresis (see, e.g., copending U.S. patent application Ser. No. 09/334,304, filed Jun. 16, 1999 (and its corresponding priority U.S. Provisional Application Serial No. 60/096,545, filed Aug. 14, 1998 and corresponding PCT Publication WO 00/76509, published Dec. 21, 2000); and U.S. patent application Ser. No. 09/665,500, filed Sep. 19, 2000, each incorporated herein by reference, and U.S. Pat. No. 5,624,677 to El-Rashidy et al. and Heaton et al., Urology, 45, 200-206, 1995).

The specific mechanisms by which apomorphine acts to produce an erectile response in a human patient are not yet completely understood but are believed to be centrally acting through dopamine receptor stimulation in the medial preoptic area of the brain. However, the dose needed to produce high enough levels in the CSF to produce an erection has often been accompanied with nausea, vomiting, hypotension and syncope. In addition, the sublingual dosage form is associated with low bioavailability compared to the subcutaneous injection.

Apomorphine previously has been shown to have very poor oral bioavailability. See, for example, Baldessarini et al., in Gessa et al., (eds.), Apomorphine and Other Dopaminomimetics, Basic Pharmacology, 1, 219-228, Raven Press, N.Y. (1981). This is another aspect of the long felt need for formulations that provide better delivery to the CSF.

Recently, results have been reported on the intranasal application of apomorphine in patients with Parkinson's disease to relieve “off-period” symptoms in patients with response fluctuations (T. van Laar et al, Arch. Neurol., 49: 482-484, 1992). The intranasally administered apomorphine reportedly used by these authors consisted of an aqueous solution of apomorphine HCl (10 mg/ml). This formulation has also used for parenteral application and is published in different Pharmacopoeia's. The reported nasal apomorphine formulation disclosed by T. van Laar et al., (1992) was:

| | | |
|---|---|---|
| Apomorphine HCl 0.5 H2O | 1 | g |
| Sodium metabisulphite | 0.100 | g |
| Sodium EDTA | 0.010 | g |
| NaCl | 0.600 | g |
| Benzalkonium Chloride | 0.01% | |
| NaH.sub.2 PO.sub.4.2H.sub.2 O | 0.150 | g |
| Na.sub.2 HPO.sub.4.2H.sub.2 O | 0.050 | g |
| NaOH 1 M to adjust pH at | | |
| 5.8 purified water to 100 ml | | |

(from Pharm. Weekblad 1991; 126: 1113-1114)

The above formulation was reportedly administered by a metered dose nebulizer in a dose of 1 mg apomorphine HCl (0.1 ml of the solution) delivered with each nasal application by puff to the patients.

Notably, the formulation reported by van Laar and coworkers would possess a major deficiency for pharmaceutical use in terms if its inherent instability. In particular, the above formulation was replicated herein and was observed to turn green, indicative of oxidation of the apomorphine, within days of preparation. It therefore does not provide a sufficiently stable formulation to be useful for pharmaceutical use.

Additional disclosures presented by Merkus et al. (U.S. Pat. No. 5,756,483) and Illum (PCT publication WO 99/27905) also report formulations of apomorphine. Comparative formulations for side-by-side tests were made in accordance with the disclosures of Merkus et al. and Illum, from which the closest formulations were ascertained and made as described in Example 3, columns 5-6 of Merkus et al., and in Example 4, page23 of Illum, respectively.

Experiments conducted herein provide detailed stability analyses of apomorphine formulations reported by Merkus et al. and Illum. To complete one analysis, the following formulation described in Example 3, columns 5-6 of Merkus et al., was made and tested in accordance with the teachings of the reference:

| 1 | Apomorphine HCl, USP | 1 | g |
|---|---|---|---|
| 2 | Methylated-β-Cylclodextrin | 4 | g |
| 3 | Sodium Metabisulfite | 0.15% | |
| 4 | Sodium EDTA | 0.1% | |
| 5 | Benzalkonium Chloride | 0.01% | |
| 6 | NaCl | 0.8% | |
| 7 | pH adjusted | to 5.5 | |
| 8 | purified water | to 100 | ml |

The formulation of Merkus et al., described above, oxidized to a green color within ten days of preparation. Accordingly, this formulation is considered unstable and not suitable for pharmaceutical use.

Additional analyses were conducted involving production and testing of the apomorphine formulation described in Example 4, page 23 of Illum. Specifically, an aqueous solution of apomorphine at a concentration of 5% w/v was mixed and pH adjusted to pH 7. However, the solution almost immediately oxidized to a green color even before the prepared microspheres could be added. Accordingly, this formulation is also considered unstable and not suitable for commercial pharmaceutical use.

In addition to these deficiencies, the Merkus disclosure cited above, relies on a very narrow dosage range of apomorphine that is specifically tailored for the treatment of the “off-period” symptoms of Parkinson's disease. In additional publications (see, e.g., U.S. Pat. No. 5,770,606 issued to El-Rashidy et al.) effective delivery of apomorphine for alleviating psychogenic impotence or erectile dysfunction is reportedly best achieved in a sublingual dosage unit. The El-Rashidy et al. disclosure includes results from a study conducted by the inventors on the effect of apomorphine delivered intranasally on erectile dysfunction. The conclusions that followed this study suggested that intranasal delivery of apomorphine at concentrations of 2.5 mg to 3.5 mg yielded extensive and serious side effects, including hypotension, nausea, vomiting, impaired vision, diaphoresis and ashen coloring. On this basis, the researchers concluded that intranasal delivery of apomorphine to treat erectile dysfunction was insufficiently safe and reliable to be a viable commercial product.

Accordingly, one of the purposes of the invention, among others, is to provide a safe and reliable methods and compositions for mucosal delivery of dopamine receptor agonists, including apomorphine, that provide for delivery of the drug via different mucosal routes in therapeutic amounts into the bloodstream or to other target site(s) for delivery, and which is fast acting, easily administered and causes no substantial adverse side effects, in particular adverse mucosal side effects such as mucosal irritation or tissue damage.

Although subcutaneous injection of dopamine receptor agonists (exemplified by apomorphine) can be used medically as noted above, this mode of administration in the case of apomorphine provides cerebral spinal fluid (CSF) levels of the active drug of less than 5% of the levels as found in the plasma. Since there is a strong correlation between apomorphine CSF levels and clinical motor responses (between 0.89 and 0.93 in one study; Hofstee et al., Clin Neuropharmacol., 17: 45-52, 1994, incorporated herein by reference), achieving delivery of dopamine receptor agonists at increased levels in the CSF represents an urgent unfulfilled need in the medical arts.

In summary, previous attempts to successfully deliver dopamine receptor agonists for therapeutic purposes have suffered from a number of important and confounding deficiencies. These deficiencies point to a long-standing unmet need in the art for pharmaceutical formulations and methods of administering dopamine agonists, such as apomorphine, that are stable and well tolerated and that provide enhanced delivery to the central nervous system (e.g., as measured by the CSF levels).

### BRIEF SUMMARY OF THE INVENTION

The present invention fulfills the foregoing needs and satisfies additional needs and advantages by providing novel, effective methods and compositions for mucosal delivery of dopamine receptor agonists yielding improved pharmacokinetic and pharmacodynamic results. In certain aspects of the invention, the dopamine receptor agonist is delivered mucosally along with one or more mucosal delivery-enhancing agent(s) to yield substantially increased absorption and/or bioavailability of the dopamine receptor agonist as compared to controls where the dopamine receptor agonist is administered to the same mucosal site alone or formulated according to previously disclosed teachings as described above.

The enhancement of mucosal delivery of dopamine receptor agonists according to the methods and compositions of the invention allows for the effective pharmaceutical use of these agents to treat a variety of diseases and conditions in mammalian subjects. Briefly, the methods and compositions provided herein provide for enhanced delivery of the dopamine receptor agonist across mucosal barriers to reach novel target sites for drug action in an enhanced, therapeutically effective rate or concentration of delivery. More specifically, the employment of one or more mucosal delivery-enhancing agents provided herein facilitates the effective delivery of a dopamine receptor agonist to a targeted, extracellular or cellular compartment, for example the systemic circulation, a selected cell population, tissue or organ. Exemplary targets for enhanced delivery in this context are target physiological compartments and fluids (e.g., within the cerebral spinal fluid (CSF)) or selected tissues or cells of the central nervous system (CNS)).

The enhanced delivery methods and compositions of the invention provide for therapeutically effective mucosal delivery of dopamine receptor agonists for prevention or treatment of a variety of disease and conditions in mammalian subjects. The dopamine receptor agonist can be administered via a variety of mucosal routes, for example by contacting to dopamine receptor agonist to a nasal mucosal epithelium, a bronchial or pulmonary mucosal epithelium, an oral, gastric, intestinal or rectal mucosal epithelium, or a vaginal mucosal epithelium. Typically, the methods and compositions are directed to or formulated for intranasal delivery.

In one aspect of the invention, pharmaceutical formulations suitable for mucosal administration are provided that comprise a therapeutically effective amount of dopamine receptor agonist and one or more mucosal delivery-enhancing agents as described herein, which formulation is effective in a mucosal delivery method of the invention to prevent the onset or progression of Parkinson's desease, or to alleviate one or more clinically well-recognized symptoms (including “off-peak” symptoms) of the disease in a mammalian subject.

In another aspect of the invention, pharmaceutical formulations suitable for mucosal administration are provided that comprise a therapeutically effective amount of a dopamine receptor agonist and one or more mucosal delivery-enhancing agents as described herein, which formulation is effective in a mucosal delivery method of the invention to prevent the onset or lower the incidence or severity of sexual dysfunction in a mammalian subject. In certain embodiments, the pharmaceutical formulations and methods of the invention prevent or alleviate male or female erectile dysfunction (e.g., as marked by engorgement and/or enhanced neural stimulation potential of male or female erectile tissues). In other embodiments, the pharmaceutical formulations and methods of the invention prevent or alleviate diminished sexual desire and/or a diminished ability to reach orgasm during sexual stimulation in a male or female mammalian subject.

In more detailed aspects of the invention, the methods and compositions which comprise a dopamine receptor agonist and one or more mucosal delivery-enhancing agent(s) (combined in a pharmaceutical formulation together or coordinately administered in a coordinate mucosal delivery protocol) yield a two- to five-fold increase, more typically a five- to ten-fold increase, and commonly a ten- to twenty-five- up to a fifty- one hundred-fold increase in transmucosal delivery of the dopamine receptor agonist (e.g., as alternately measured by maximal concentration (Cmax) or time to maximal concentration (tmax) in serum, cerebral spinal fluid, or in another selected physiological compartment or target tissue or organ for delivery), compared to delivery efficacy for the dopamine receptor agonist administered alone or in accordance with conventional technologies.

In exemplary embodiments, the methods and compositions of the invention yield a two- to five-fold increase, more typically a five- to ten-fold increase, and commonly a ten- to twenty-five- up to a fifty- one hundred-fold increase in a transmucosal delivery rate (tmax) of the dopamine receptor agonist in serum, cerebral spinal fluid, or in another selected physiological compartment or target tissue or organ for delivery), compared to delivery rates for the dopamine receptor agonist administered alone or in accordance with conventional technologies.

For example, pharmaceutical preparations formulated for mucosal (e.g., intranasal) delivery are provided for treating sexual dysfunction in a mammalian subject that comprise a therapeutically effective amount of a dopamine receptor agonist (e.g., apomorphine) combined with one or more mucosal delivery-enhancing agents as disclosed herein. These preparations surprisingly yield enhanced mucosal absorption of the dopamine receptor agonist to produce a therapeutic effect (e.g., an erection sufficient for vaginal penetration or yielding improved sexual arousal) in the subject in about 45 minutes or less, 30 minutes or less, 20 minutes or less, or as little as 15 minutes or less following administration of the preparation.

Other exemplary pharmaceutical preparations formulated for enhanced mucosal (e.g., intranasal) delivery according to the invention provided for a surprisingly increased rate of delivery of a dopamine receptor agonist (e.g., apomorphine) for treating a selected disease or condition in a mammalian subject, wherein a time to maximal plasma concentration (tmax) of the dopamine agonist following mucosal administration of the preparation is about 30 minutes or less, 20 minutes or less, or as little as 15 minutes or less.

In other aspects of the invention, the methods and formulations for mucosally administering a dopamine receptor agonist described herein yield a significantly enhanced rate or level of delivery (e.g., increased tmax or Cmax) of the dopamine receptor agonist into the central nervous system (CNS) of the subject. This includes enhanced delivery rates or levels into the cerebral spinal fluid (CSF), or to selected tissues or cells (e.g., a particular brain region or neuron population) of the CNS, compared to delivery rates and levels for the dopamine receptor agonist administered alone or in accordance with conventional technologies. Thus, in certain aspects of the invention, the foregoing methods and compositions are administered to a mammalian subject to yield enhanced delivery of the dopamine receptor agonist to a physiological compartment, fluid, tissue or cell within the central nervous system (CNS) of a mammalian subject.

In exemplary embodiments, administration of one or more dopamine receptor agonists formulated with one or more mucosal delivery-enhancing agents as described herein yields effective CNS delivery to alleviate a selected disease or condition (e.g., Parkinson's disease or a symptom thereof) in a mammalian subject. In more detailed aspects, the methods and formulations for mucosally administering a dopamine receptor agonist according to the invention yield a significantly enhanced rate or level of delivery (e.g., increased tmax or Cmax) of the dopamine receptor agonist into the CNS (including but not limited to enhanced delivery rates or levels into the cerebral spinal fluid (CSF)), or to selected tissues or cells (e.g., a particular brain region or neuron population) of the CNS), compared to delivery rates and levels for the dopamine receptor agonist administered alone or in accordance with conventional technologies. Within specific aspects, the enhanced delivery rate or level of the dopamine receptor agonist provides for effective treatment of sexual dysfunction or Parkinson's disease in a subject. For example, by using the mucosal adminstration methods and formulations of the invention, an effective concentration of a dopamine receptor agonist (e.g., apomorphine) can be delivered to the CSF to mediate stimulation of an erectile (increased hemodynamic or sensory) response in the subject, usually within about 45 min, 30 min, 20 min, and even 15 min or less following administration. The rate and level of delivery of the dopamine receptor agonist is effective for this and other therapeutic purposes (e.g., to alleviate off peak Parkinson's symptoms) disclosed herein, without unacceptable adverse side effects such as severe nausea, vomiting, hypotension and syncope.

Within other detailed embodiments of the invention, the foregoing methods and formulations are administered to a mammalian subject to yield enhanced CNS delivery of the dopamine receptor agonist, whereby the peak dopamine agonist concentration in a CNS target site for delivery (e.g., within the CSF or within or surrounding a selected tissue or cell population) is at least 5% of the peak dopamine agonist concentration in the blood plasma following administration of the formulation to the subject. In exemplary embodiments, administration of one or more dopamine receptor agonists formulated with one or more mucosal delivery-enhancing agents as described herein yields a peak dopamine agonist concentration in the CSF of about 5-10% or greater versus the peak dopamine agonist concentration in the blood plasma following administration of the formulation to the subject. In other exemplary embodiments, the peak dopamine agonist concentration in the CSF is about 15% or greater versus the peak dopamine agonist concentration in the blood plasma. In yet additional exemplary embodiments, the peak dopamine agonist concentration in the CSF is about 20% or greater, 30% or greater, 35% or greater, or up to 40% or greater, versus the peak dopamine agonist concentration in the blood plasma. These enhanced rates and levels of delivery are correlated directly with the efficacy of the mucosal delivery methods and formulations of the invention for prophylaxis and treatment of diseases and conditions in mammalian subjects amenable to prophylaxis and treatment by CNS delivery of therapeutic levels of selected dopamine receptor agonists.

The foregoing mucosal drug delivery formulations and preparative and delivery methods of the invention provide for improved mucosal delivery of dopamine receptor agonists to mammalian subjects. These compositions and methods can involve combinatorial formulation or coordinate administration of one or more dompamine receptor agonist(s) with one or more mucosal delivery-enhancing agents. Among the mucosal delivery-enhancing agents to be selected from to achieve these formulations and methods are (a) aggregation inhibitory agents; (b) charge modifying agents; (c) pH control agents; (d) degradative enzyme inhibitors; (e) mucolytic or mucus clearing agents; (f) ciliostatic agents; (g) membrane penetration-enhancing agents (e.g., (i) a surfactant, (ii) a bile salt, (ii) a phospholipid or fatty acid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a clyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, (x) an inhibitor of cholesterol synthesis; or (xi) any combination of the membrane penetration enhancing agents of (i)-(x)); (h) modulatory agents of epithelial junction physiology, such as nitric oxide (NO) stimulators, chitosan, and chitosan derivatives; (i) vasodilator agents; (j) selective transport-enhancing agents; and (k) stabilizing delivery vehicles, carriers, supports or complex-forming species with which the dopamine receptor agonist(s) is/are effectively combined, associated, contained, encapsulated or bound to stabilize the active agent for enhanced mucosal delivery.

In various embodiments of the invention, one or more dopamine receptor agonist(s) is/are combined with one, two, three, four or more of the mucosal delivery-enhancing agents recited in (a)-(k), above. These delivery-enhancing agents may be admixed, alone or together, with the dopamine receptor agonist, or otherwise combined therewith in a pharmaceutically acceptable formulation or delivery vehicle. Formulation of a dopamine receptor agonist with one or more of the mucosal delivery-enhancing agents according to the teachings herein (optionally including any combination of two or more delivery-enhancing agents selected from (a)-(k) above) provides for increased bioavailability of the dopamine receptor agonist following delivery thereof to a mucosal surface of a mammalian subject.

In related aspects of the invention, a variety of coordinate administration methods are provided for enhanced mucosal delivery of a dopamine receptor agonist, such as apomorphine. These methods comprise the step, or steps, of administering to a mammalian subject a mucosally effective amount of at least one dopamine receptor agonist in a coordinate administration protocol with one or more mucosal delivery-enhancing agents selected from (a) aggregation inhibitory agents; (b) charge modifying agents; (c) pH control agents; (d) degradative enzyme inhibitors; (e) mucolytic or mucus clearing agents; (f) ciliostatic agents; (g) membrane penetration-enhancing agents (e.g., (i) a surfactant, (ii) a bile salt, (ii) a phospholipid or fatty acid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a clyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, (x) an inhibitor of cholesterol synthesis; or (xi) any combination of the membrane penetration enhancing agents of (i)-(x)); (h) modulatory agents of epithelial junction physiology, such as nitric oxide (NO) stimulators, chitosan, and chitosan derivatives; (i) vasodilator agents; (j) selective transport-enhancing agents; and (k) stabilizing delivery vehicles, carriers, supports or complex-forming species with which the dopamine receptor agonist(s) is/are effectively combined, associated, contained, encapsulated or bound to stabilize the active agent for enhanced mucosal delivery. To practice a coordinate administration method according to the invention, any combination of one, two or more of the mucosal delivery-enhancing agents recited in (a)-(k), above, may be admixed or otherwise combined for simultaneous mucosal administration. Alternatively, any combination of one, two or more of the intranasal delivery-enhancing agents recited in (a)-(k) can be mucosally administered, collectively or individually, in a predetermined temporal sequence separated from mucosal administration of the dopamine receptor agonist (e.g., by pre-administering one or more of the delivery-enhancing agent(s)), and via the same or different delivery route as the dopamine receptor agonist (e.g., to the same or to a different mucosal surface as the dopamine receptor agonist, or even via a non-mucosal (e.g., subcutaneous, or intravenous) route). Coordinate administration of dopamine receptor agonists with any one, two or more of the mucosal delivery-enhancing agents according to the teachings herein provides for increased bioavailability of the dopamine receptor agonists following delivery thereof to a mucosal surface of a mammalian subject.

In additional related aspects of the invention, various “multi-processing” or “co-processing” methods are provided for preparing formulations of dopamine receptor agonists for for enhanced mucosal delivery. These methods comprise one or more processing or formulation steps wherein one or more dopamine receptor agonist(s) is/are serially, or simultaneously, contacted with, reacted with, or formulated with, one, two or more (including any combination of) of the mucosal delivery-enhancing agents selected from (a) aggregation inhibitory agents; (b) charge modifying agents; (c) pH control agents; (d) degradative enzyme inhibitors; (e) mucolytic or mucus clearing agents; (f) ciliostatic agents; (g) membrane penetration-enhancing agents (e.g., (i) a surfactant, (ii) a bile salt, (ii) a phospholipid or fatty acid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a clyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, (x) an inhibitor of cholesterol synthesis; or (xi) any combination of the membrane penetration enhancing agents of (i)-(x)); (h) modulatory agents of epithelial junction physiology, such as nitric oxide (NO) stimulators, chitosan, and chitosan derivatives; vasodilator agents; (j) selective transport-enhancing agents; and (k) stabilizing delivery vehicles, carriers, supports or complex-forming species with which the dopamine receptor agonist(s) is/are effectively combined, associated, contained, encapsulated or bound to stabilize the dopamine receptor agonist for enhanced mucosal delivery.

To practice the multi-processing or co-processing methods according to the invention, the dopamine receptor antagonist(s) is/are exposed to, reacted with, or combinatorially formulated with any combination of one, two or more of the mucosal delivery-enhancing agents recited in (a)-(k), above, either in a series of processing or formulation steps, or in a simultaneous formulation procedure, that modifies the dopamine receptor agonist (or other formulation ingredient) in one or more structural or functional aspects, or otherwise enhances mucosal delivery of the active agent in one or more (including multiple, independent) aspect(s) that are each attributed, at least in part, to the contact, modifying action, or presence in a combinatorial formulation, of a specific mucosal delivery-enhancing agent recited in (a)-(k), above.

In certain detailed aspects of the invention, a stable pharmaceutical formulation is provided which comprises a dopamine receptor agonist and one or more delivery-enhancing agent(s), wherein the formulation administered mucosally to a mammalian subject yields a peak concentration of the dopamine receptor agonist in a central nervous system tissue or fluid (e.g., cerebral spinal fluid) of the subject that is 5% or greater compared to a peak concentration of the dopamine receptor agonist in a blood plasma (e.g., venous serum) of the subject. Often the formulation is administered to a nasal mucosal surface of the subject. In certain embodiments, the dopamine receptor agonist is apomorphine or a pharmaceutically acceptable salt or derivative thereof. An effective dose of the dopamine receptor agonist is, for example, between about 0.25 and 2.0 mg.

In certain embodiments of the invention, the mucosal formulation of the dopamine receptor agonist(s) and one or more delivery-enhancing agent(s) yields a peak dopamine receptor agonist concentration in a cerebral spinal fluid of the subject that is between about 5-10% of the peak dopamine receptor agonist concentration in the blood plasma of the subject. Alternately, the formulation yields a peak dopamine receptor agonist concentration in the cerebral spinal fluid that is about 10%, 15%, 20%, 25%, 30%, 35%, 40%, or greater compared to the peak dopamine receptor agonist concentration in the blood plasma. Typically, mucosal administration of the formulation yields a peak concentration of the dopamine receptor agonist in the central nervous system tissue or fluid of the subject that is greater than a peak concentration of the dopamine receptor agonist in the central nervous system tissue or fluid of the subject following injection of the same concentration or dose of the dopamine receptor agonist.

### BRIEF DESCRIPTION OF THE DRAWING

FIG. 1 provides a schematic flow illustration summarizing the synthesis of β-[1→4]-2-guanidino-2-deoxy-D-glucose polymer (poly-GuD), a novel chitosan derivative for use within certain mucosal delivery formulations and methods of the invention.

### DETAILED DESCRIPTION OF THE INVENTION

As noted above, the present invention provides improved methods and compositions for mucosal delivery of dopamine receptor agonists to mammalian subjects for treatment or prevention of a variety of diseases and conditions. Examples of appropriate mammalian subjects for treatment and prophylaxis according to the methods of the invention include, but are not restricted to, humans and non-human primates, livestock species, such as horses, cattle, sheep, and goats, and research and domestic species, including dogs, cats, mice, rats, guinea pigs, and rabbits.

In order to provide better understanding of the present invention, the following definitions are provided:

Dopamine receptor agonists: In the central nerous system, dopaminergic neurotransmission is mediated bthrough receptors belonging to the G protein-coupled receptor family. On the basis of their structural homology, several different types of dopamine receptors have geen identified and cloned, the most abundant of which are termed D1 and D2 dopaminergic receptors. Recently, three other types of dopamine receptors, D3, D4, and D5, have been identified and found to be expresssed in different areas of the brain. The affinity of these different receptors for dopamine also varies significantly. As used herein, “dopamine receptor agonists” include all natural and synthetic agents that function as specific agonists acting directly on striatal dopamine receptors. Many such agonists are well known in the art and readily available for use within the methods and compostions of the inveniton. Natural and synthetic or semisynthetic ergolines derived or modeled after ergot alkyloids comprise a principal class of dopamine receptor agonists for use within the invention. Representative dopamine receptor agonists in this regard, include by way of illustration and not limitation, apomorphines and ergotamines. Specific examples or dopamine receptor agonists for use within the invention include, but are not limited to, levodopa/carbidopa, amantadine, bromocriptine, pergolide, apomorphine, benserazide, lysuride, mesulergine, lisuride, lergotrile, memantine, metergoline, piribedil, tyramine, tyrosine, phenylalanine, bromocriptine mesylate, pergolide mesylate, and the like. In certain embodiments of the invention, the dopamine receptor agonist acts on one or more specific dopamine receptors. A number of tetralins and related ergoline derivatives have been reported as centrally acting D2 dopamine receptor agonists. (Wickstrom, Prog. Med. Chem. 29:185-216, 1992 (incorporated herein by reference). Among additional compounds that have been tested for receptor specificity, 5-hydroxy-2-N,N-n-dipropylaminotetralin (5-OH-DPAT), 7-OH-DPAT and 8-OH-DPAT, are reported as specific and selective ligands for the D3 receptors (Levesque, Proc. Natl. Acad. Sci. USA 89:8155-8159, 1992; Mulder, et al., Arch. Pharmacol. 336: 494-501, 1987; and Beart, et al, Arch. Pharmacol. 336: 487-493, 1987, each incorporated herein by reference).

Additional disclosures teach detailed methods and tools pointing to specific structural and functional characteristics that define effective dopamine receptor agonists, and further disclose a diverse, additional array of these agents that are useful within the invention. Thus, for example, Muralikrishnan, Brain Res. 892:241-7, 2001 (incorporated herein by reference) describes a D1 dopaminergic receptor agonist, SKF-38393 HCl (SKF). Reaville et al., J. Pharm. Pharmacol. 52:1129-35, 2000 (incorporated herein by reference), describe a related agonist, ropinirole (SKF-101468). Self et al., Ann. N Y Acad. Sci. 909:133-44, 2000 (incorporated herein by reference) teach a novel D1 agonist ABT-431 that is also useful within the invention. Additional teachings regarding identification, selection, pharmacology, and production of dopamine receptor agonists and their diverse assemblage of derivatives and analogs for employment within the methods and compositions of the invention, are provided, for example, by DeWald et al., J. Med. Chem. 33:445-450, 1990; Grol et al., J. Pharm. Pharmacol. 43:481-485, 1991; Hall et al., J. Med. Chem. 30:1879-1887, 1987; Horn et al., J. Med. Chem. 27: 1340-1343, 1984; Johansson et al., J. Med. Chem. 30: 1827-1837, 1987; Jobansson et al., Mol. Pharmacol. 30:258-269, 1986; Johansson et al., J. Med. Chem. 28:1049-1053, 1985; Johansson et al., J. Med. Chem. 30:602-611, 1987; Johansson et al., J. Org. Chem. 51: 5252-5258, 1986; Johansson et al., J. Med. Chem. 33:2925-2929, 1990; Jones et al., J. Med. Chem. 27:1607-1613; 1984; Langlois et al., Synthetic Comm. 22:1723-1734, 1992; Martin et al., J. Pharmacol. Exp. Ther. 230:569-576, 1989; Neumeyer et al., J. Med. Chem. 34:24-28, 1991; Seiler et al., Mol. Pharmacol. 22:281-289, 1982; and Sibley et al., TIPS 13: 61-68, 1992 (each incorporated herein by reference).

The term dopamine receptor agonists as used herein also embraces chemically modified analogs, derivatives, salts and esters of dopamine receptor agonists which are “pharmaceutically acceptable,” for example salts and esters of dopamine receptor agonists that are suitable for use in contact with mucosal tissues of humans and other mammals, without undue toxicity, irritation, allergic response, and the like, and which retain activity for their intended use, such as for chemotherapy and prophylaxis of dopamine deficiency associated with Parkinson's disease. Pharmaceutically acceptable salts of dopamine receptor agonists can be prepared in situ during isolation and purification of dopamine agonists, or separately by reacting the free base or acid functions of the dopamine receptor agonist with a suitable organic acid or base. Representative acid addition salts include the hydrochloride, hydrobromide, sulphate, bisulphate, acetate, oxalate, valerate, oleate, palmitate, stearate, laurate, borate, benzoate, lactate, phosphate, tosylate, mesylate, citrate, maleate, fumarate, succinate, tartrate, ascorbate, glucoheptonate, lactobionate, lauryl sulphate salts and the like. Representative alkali or alkaline earth metal salts include the sodium, calcium, potassium and magnesium salts, and the like.

The term “apomorphine” as used herein includes the free base form of this compound as well as all pharmacologically acceptable analogs, deriviatives, and chemically modified forms, including acid addition salts, thereof. In addition to the hydrochloride salt, other acceptable acid addition salts are the hydrobromide, the hydroiodide, the bisulfate, the phosphate, the acid phosphate, the lactate, the citrate, the tartarate, the salicylate, the succinate, the maleate, the gluconate, and the like.

Treatment and Prevention of Sexual Dysfunction: As noted above, the instant invention provides useful methods and compositions to prevent and treat sexual dysfunction in mammalian subjects. As used herein, prevention and treatment of sexual dysfunction mean prevention of the onset or lowering the incidence or severity of sexual dysfunction in a mammalian subject. In certain embodiments, the pharmaceutical formulations and methods of the invention prevent or alleviate male or female erectile dysfunction. Erectile dysfunction in one regard means a failure or reduction of hemodynamic responsiveness in a subject (e.g., as compared to a normal response in a suitable control subject) leading to penile or clitoral intracavernosal engorgement or engorgement of the vaginal wall or other genital tissues subject to hemodynamic engorgement during sexual stimulation. This failure or reduced response may mediated by reduced neural stimulation (e.g., via the vaginal/clitoral or penile branch of the pelvic nerve) of genital or peri-genital tissues that normally mediate an erectile response, which can in turn yield dysfunction in the level of sexual sensitivity in a subject, or in terms of failure or reduction of the hemodynamic erectile response. Thus, prevention or alleviation of sexual dysfunction according using the methods and compositions of the invention can involve, or be determined by, an increase in neural stimulation to genital or peri-genital tissues, an increased level of sexual desire or arousal, an increased erectile response (e.g., as measured by blood flow in an erectile tissue, degree of penile engorgement and suitability for vaginal penetration, duration of erectile response, and associated sensory stimulation levels achieved or expressed by a subject) or an increased ability to reach orgasm during sexual stimulation in a male or female mammalian subject. Encompassed within the term sexual dysfunction are therefore conditions commonly referred to as impotence, decreased sexual desire, decreased sexual arousal, dyspareunia, and/or difficulty or inability to achieve orgasm.

Within the mucosal delivery formulations and methods of the invention, the dopamine receptor agonist is frequently combined or coordinately administered with a suitable carrier or vehicle for mucosal delivery. As used herein, the term “carrier” means a pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories, can be found in the U.S. Pharmacopeia National Formulary, pp. 1857-1859, 1990, which is incorporated herein by reference. Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other non toxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator. Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like. The amount of active ingredient that can be combined with the carrier materials to produce a single dosage form will vary depending upon the particular mode of administration.

The mucosal formulations of the invention are generally sterile, particulate free and stable for pharmaceutical use. As used herein, the term “particulate free” means a formulation that meets the requirements of the USP specification for small volume parenteral solutions. The term “stable” means a formulation that fulfills all chemical and physical specifications with respect to identity, strength, quality, and purity which have been established according to the principles of Good Manufacturing Practice, as set forth by appropriate governmental regulatory bodies.

Within the mucosal delivery compositions and methods of the invention, various delivery-enhancing agents are employed which enhance delivery of a dopamine receptor agonist into or across a mucosal surface. In this regard, delivery of dopamine receptor agonists across the mucosal epithelium can occur “transcellularly” or “paracellularly”. The extent to which these pathways contribute to the overall flux and bioavailability of the dopamine receptor agonist depends upon the environment of the mucosa, the physico-chemical properties the active agent, and on the properties of the mucosal epithelium. Paracellular transport involves only passive diffusion, whereas transcellular transport can occur by passive, facilitated or active processes. Generally, hydrophilic, passively transported, polar solutes diffuse through the paracellular route, while more lipophilic solutes use the transcellular route. Absorption and bioavailability (e.g., as reflected by a permeability coefficient or physiological assay), for diverse, passively and actively absorbed solutes, can be readily evaluated, in terms of both paracellular and transcellular delivery components, for any selected dopamine receptor agonist within the invention. These values can be determined and distinguished according to well known methods, such as in vitro epithelial cell culture permeability assays (see, e.g., Hilgers, et al., Pharm. Res.7:902-910, 1990; Wilson et al., J. Controlled Release 11 :25-40, 1990; Artursson. I., Pharm. Sci. 79:47682, 1990; Cogburn et al., Pharm. Res. 8:210-216, 1991; Pade et al., Pharmaceutical Research 14:1210-1215, 1997, each incorporated herein by reference).

For passively absorbed drugs, the relative contribution of paracellular and transcellular pathways to drug transport depends upon the pKa, partition coefficient, molecular radius and charge of the drug, the pH of the luminal environment in which the drug is delivered, and the area of the absorbing surface. The paracellular route represents a relatively small fraction of accessible surface area of the nasal mucosal epithelium. In general terms, it has been reported that cell membranes occupy a mucosal surface area that is a thousand times greater than the area occupied by the paracellular spaces. Thus, the smaller accessible area, and the size- and charge-based discrimination against macromolecular permeation would suggest that the paracellular route qould be a generally less favorable route than transcellular delivery for drug transport. Surprisingly, the methods and compositions of the invention provide for significantly enhanced transport of biotherapeutics into and across mucosal epithelia via the paracellular route. Therefore, the methods and compositions of the invention successfully target both paracellular and transcellular routes, alternatively or within a single method or composition.

As used herein, “mucosal delivery-enhancing agents” include agents which enhance the release or solubility (e.g., from a formulation delivery vehicle), diffusion rate, penetration capacity and timing, uptake, residence time, stability, effective half-life, peak or sustained concentration levels, clearance and other desired mucosal delivery characteristics (e.g., as measured at the site of delivery, or at a selected target site of activity such as the bloodstream or central nervous system) of a dopamine receptor agonist or other biologically active compound(s). Enhancement of mucosal delivery can thus occur by any of a variety of mechanisms, for example by increasing the diffusion, transport, persistence or stability of dopamine receptor agonists, increasing membrane fluidity, modulating the availability or action of calcium and other ions that regulate intracellular or paracellular permeation, solubilizing mucosal membrane components (e.g., lipids), changing non-protein and protein sulfhydryl levels in mucosal tissues, increasing water flux across the mucosal surface, modulating epithelial junctional physiology, reducing the viscosity of mucus overlying the mucosal epithelium, reducing mucociliary clearance rates, and other mechanisms.

Mucosal delivery of dopamine receptor agonists to a target site for drug activity in the subject may involve a variety of delivery or transfer routes. For example, a given active agent may find its way through clearances between cells of the mucosa and reach an adjacent vascular wall, while by another route the agent may, either passively or actively, be taken up into mucosal cells to act within the cells or be discharged or transported out of the cells to reach a secondary target site, such as the systemic circulation. The methods and compositions of the invention may promote the translocation of active agents along one or more such alternate routes, or may act directly on the mucosal tissue or proximal vascular tissue to promote absorption or penetration of the active agent(s). The promotion of absorption or penetration in this context is not limited to these mechanisms.

Many known reagents that are reported to enhance mucosal absorption also cause irritation or damage to mucosal tissues (see, e.g., Swenson and Curatolo, Adv. Drug Delivery Rev. 8:39-92, 1992, incorporated herein by reference). For example, in studies of intestinal absorption enhancing agents, the delivery-enhancing effects of various absorption-promoting agents are reportedly directly related to their membrane toxicity (see, e.g., Uchiyama et al., Biol. Pharm. Bull. 19:1618-1621, 1996; Yamamoto et al., J. Pharm. Pharmacol. 48:1285-1289, 1996, each incorporated herein by reference). In this regard, the combinatorial formulation and coordinate administration methods of the present invention incorporate effective, minimally toxic delivery-enhancing agents to enhance mucosal delivery of dopamine receptor agonist and other biologically active macromolecules useful within the invention.

While the mechanism of absorption promotion may vary with different delivery-enhancing agents of the invention, useful reagents in this context will not substantially adversely affect the mucosal tissue and will be selected according to the physicochemical characteristics of the particular dopamine receptor agonist or other active or delivery-enhancing agent. In this context, delivery enhancing agents that increase penetration or permeability of mucosal tissues will often result in some alteration of the protective permeability barrier of the mucosa. For such delivery-enhancing agents to be of value within the invention, it is generally desired that any significant changes in permeability of the mucosa be reversible within a time frame appropriate to the desired duration of drug delivery. Furthermore, there should be no substantial, cumulative toxicity, nor any permanent deleterious changes induced in the barrier properties of the mucosa with long-term use.

Within certain aspects of the invention, absorption-promoting agents for coordinate administration or combinatorial formulation with dopamine receptor agonists of the invention are selected from small hydrophilic molecules, including but not limited to, dimethyl sulfoxide (DMSO), dimethylformamide, ethanol, propylene glycol, and the 2-pyrrolidones. Alternatively, long-chain amphipathic molecules, for example, deacylmethyl sulfoxide, azone, sodium laurylsulfate, oleic acid, and the bile salts, may be employed to enhance mucosal penetration of the dopamine receptor agonist. In additional aspects, surfactants (e.g., polysorbates) are employed as adjunct compounds, processing agents, or formulation additives to enhance intranasal delivery of the dopamine receptor agonist. These penetration enhancing agents typically interact at either the polar head groups or the hydrophilic tail regions of molecules which comprise the lipid bilayer of epithelial cells lining the nasal mucosa (Barry, Pharmacology of the Skin, Vol. 1, pp. 121-137, Shroot et al., Eds., Karger, Basel, 1987; and Barry, J. controlled Release 6:85-97, 1987, each incorporated herein by reference). Interaction at these sites may have the effect of disrupting the packing of the lipid molecules, increasing the fluidity of the bilayer, and facilitating transport of the dopamine receptor agonist across the mucosal barrier. Interaction of these penetration enhancers with the polar head groups may also cause or permit the hydrophilic regions of adjacent bilayers to take up more water and move apart, thus opening the paracellular pathway to transport of the dopamine receptor agonist. In addition to these effects, certain enhancers may have direct effects on the bulk properties of the aqueous regions of the nasal mucosa. Agents such as DMSO, polyethylene glycol, and ethanol can, if present in sufficiently high concentrations in delivery environment (e.g., by pre-administration or incorporation in a therapeutic formulation), enter the aqueous phase of the mucosa and alter its solubilizing properties, thereby enhancing the partitioning of the dopamine receptor agonist from the vehicle into the mucosa.

Additional delivery-enhancing agents that are useful within the coordinate administration and processing methods and combinatorial formulations of the invention include, but are not limited to, mixed micelles; enamines; nitric oxide donors (e.g., S-nitroso-N-acetyl-DL-penicillamine, NOR1,NOR4—which are preferably co-administered with an NO scavenger such as carboxy-PITO or doclofenac sodium); sodium salicylate; glycerol esters of acetoacetic acid (e.g., glyceryl-1,3-diacetoacetate or 1,2-isopropylideneglycerine-3-acetoacetate); and other release-diffusion or intra- or trans-epithelial penetration-promoting agents that are physiologically compatible for mucosal delivery. Other absorption-promoting agents are selected from a variety of carriers, bases and excipients that enhance mucosal delivery, stability, activity or trans-epithelial penetration of the dopamine receptor agonist. These include, inter alia, clyclodextrins and beta-cyclodextrin derivatives (e.g., 2-hydroxypropyl-beta-cyclodextrin and heptakis(2,6-di-O-methyl-beta-cyclodextrin). These compounds, optionally conjugated with one or more of the active ingredients and further optionally formulated in an oleaginous base, enhance bioavailability in the mucosal formulations of the invention. Yet additional absorption-enhancing agents adapted for mucosal delivery include medium-chain fatty acids, including mono- and diglycerides (e.g., sodium caprate—extracts of coconut oil, Capmul), and triglycerides (e.g., amylodextrin, Estaram 299, Miglyol 810).

The mucosal therapeutic and prophylactic compositions of the present invention may be supplemented with any suitable penetration-promoting agent that facilitates absorption, diffusion, or penetration of dopamine receptor agonists across mucosal barriers. The penetration promoter may be any promoter that is pharmaceutically acceptable. Thus, in more detailed aspects of the invention compositions are provided that incorporate one or more penetration-promoting agents selected from sodium salicylate and salicylic acid derivatives (acetyl salicylate, choline salicylate, salicylamide, etc.); amino acids and salts thereof (e.g. monoaminocarboxlic acids such as glycine, alanine, phenylalanine, proline, hydroxyproline, etc.; hydroxyamino acids such as serine; acidic amino acids such as aspartic acid, glutamic acid, etc; and basic amino acids such as lysine etc—inclusive of their alkali metal or alkaline earth metal salts); and N-acetylamino acids (N-acetylalanine, N-acetylphenylalanine, N-acetylserine, N-acetylglycine, N-acetyllysine, N-acetylglutamic acid, N-acetylproline, N-acetylhydroxyproline, etc.) and their salts (alkali metal salts and alkaline earth metal salts). Also provided as penetration-promoting agents within the methods and compositions of the invention are substances which are generally used as emulsifiers (e.g. sodium oleyl phosphate, sodium lauryl phosphate, sodium lauryl sulfate, sodium myristyl sulfate, polyoxyethylene alkyl ethers, polyoxyethylene alkyl esters, etc.), caproic acid, lactic acid, malic acid and citric acid and alkali metal salts thereof, pyrrolidonecarboxylic acids, alkylpyrrolidonecarboxylic acid esters, N-alkylpyrrolidones, proline acyl esters, and the like.

Within various aspects of the invention, improved mucosal delivery formulations and methods are provided that allow delivery of dopamine receptor agonists and other therapeutic agents within the invention across mucosal barriers between administration and selected target sites. Certain formulations are specifically adapted for a selected target cell, tissue or organ, or even a particular disease state. In other aspects, formulations and methods provide for efficient, selective endo- or transcytosis of a dopamine receptor agonist specifically routed along a defined intracellular or intercellular pathway. Typically, the dopamine receptor agonist is efficiently loaded at effective concentration levels in a carrier or other delivery vehicle, and is delivered and maintained in a stabilized form, e.g., at the nasal mucosa and/or during passage through intracellular compartments and membranes to a remote target site for drug action (e.g., the blood stream or a defined tissue, organ, or extracellular compartment). The dopamine receptor agonist may be provided in a delivery vehicle or otherwise modified (e.g., in the form of a prodrug), wherein release or activation of the dopamine receptor agonist is triggered by a physiological stimulus (e.g. pH change, lysosomal enzymes, etc.) Often, the dopamine receptor agonist is pharmacologically inactive until it reaches its target site for activity. In most cases, the dopamine receptor agonist and other formulation components are non-toxic and non-immunogenic. In this context, carriers and other formulation components are generally selected for their abilitity to be rapidly degraded and excreted under physiological conditions. At the same time, formulations are chemically and physically stable in dosage form for effective storage.

Other Biologically Active Agents

The methods and compositions of the present invention are directed toward enhancing mucoal delivery of dopamine receptor agonists, but are also useful for enhancing mucosal delivery of a broad spectrum of additional biologically active agents to achieve therapeutic, prophylactic or other physiological results in mammalian subjects. As used herein, the term “biologically active agent” encompasses any substance that produces a physiological response when mucosally administered to a mammalian subject according to the methods and compositions herein. Useful biologically active agents in this context include therapeutic or prophylactic agents applied in all major fields of clinical medicine, as well as nutrients, cofactors, enzymes (endogenous or foreign), antioxidants, and the like. Thus, the biologically active agent may be water-soluble or water-insoluble, and may include higher molecular weight proteins, peptides, carbohydrates, glycoproteins, lipids, and/or glycolipids, nucleosides, polynucleotides, and other active agents.

Useful pharmaceutical agents within the methods and compositions of the invention include drugs and macromolecular (high molecular weight) therapeutic or prophylactic agents embracing a wide spectrum of compounds, including small molecule drugs, peptides, proteins, and vaccine agents. Exemplary pharmaceutical agents for use within the invention are biologically active for treatment or prophylaxis of a selected disease or condition in the subject. Biological activity in this context can be determined as any significant (i.e., measurable, statistically significant) effect on a physiological parameter, marker, or clinical symptom associated with a subject disease or condition, as evaluated by an appropriate in vitro or in vivo assay system involving actual patients, cell cultures, sample assays, or acceptable animal models.

The methods and compositions of the invention provide unexpected advantages for treatment of diseases and other conditions in mammalian subjects, which advantages are mediated, for example, by providing enhanced speed, duration, fidelity or control of intranasal delivery of therapeutic and prophylactic compounds to reach selected physiological compartments in the subject (e.g., into or across the nasal mucosa, into the systemic circulation or central nervous system (CNS), or to any selected target organ, tissue, fluid or cellular or extracellular compartment within the subject).

In various exemplary embodiments, the methods and compositions of the invention may incorporate one or more biologically active agent(s) in addition to a dopamine receptor agonist, selected from:

opiods or opiod antagonists, such as morphine, hydromorphone, oxymorphone, lovorphanol, levallorphan, codeine, nalmefene, nalorphine, nalozone, naltrexone, buprenorphine, butorphanol, and nalbufine;

corticosterones, such as cortisone, hydrocortisone, fludrocortisone, prednisone, prednisolone, methylprednisolone, triamcinolone, dexamethoasone, betamethoasone, paramethosone, and fluocinolone;

other anti-inflammatories, such as colchicine, ibuprofen, indomethacin, and piroxicam; anti-viral agents such as acyclovir, ribavarin, trifluorothyridine, Ara-A (Arabinofuranosyladenine), acylguanosine, nordeoxyguanosine, azidothymidine, dideoxyadenosine, and dideoxycytidine; antiandrogens such as spironolactone;

androgens, such as testosterone;

estrogens, such as estradiol;

progestins;

muscle relaxants, such as papaverine;

vasodilators, such as nitroglycerin, vasoactive intestinal peptide and calcitonin related gene peptide;

antihistamines, such as cyproheptadine;

agents with histamine receptor site blocking activity, such as doxepin, imipramine, and cimetidine;

antitussives, such as dextromethorphan; neuroleptics such as clozaril; antiarrhythmics;

antiepileptics;

enzymes, such as superoxide dismutase and neuroenkephalinase;

anti-fungal agents, such as amphotericin B, griseofulvin, miconazole, ketoconazole, tioconazol, itraconazole, and fluconazole;

antibacterials, such as penicillins, cephalosporins, tetracyclines, aminoglucosides, erythromicin, gentamicins, polymyxin B;

anti-cancer agents, such as 5-fluorouracil, bleomycin, methotrexate, and hydroxyurea, dideoxyinosine, floxuridine, 6-mercaptopurine, doxorubicin, daunorubicin, I-darubicin, taxol, and paclitaxel;

antioxidants, such as tocopherols, retinoids, carotenoids, ubiquinones, metal chelators, and phytic acid;

antiarrhythmic agents, such as quinidine; and

antihypertensive agents such as prazosin, verapamil, nifedipine, and diltiazem; analgesics such as acetaminophen and aspirin;

monoclonal and polyclonal antibodies, including humanized antibodies, and antibody fragments;

anti-sense oligonucleotides; and

RNA, DNA and viral vectors comprising genes encoding therapeutic peptides and proteins.

In addition to these exemplary classes and species of active agents, the methods and compositions of the invention embrace any physiologically active agent, as well as any combination of multiple active agents, described above or elsewhere herein or otherwise known in the art, that is individually or combinatorially effective within the methods and compositions of the invention for treatment or prevention of a selected disease or condition in a mammalian subject (see, Physicians' Desk Reference, published by Medical Economics Company, a division of Litton Industries, Inc, incorporated herein by reference).

Regardless of the class of compound employed, the biologically active agent for use within the invention will be present in the composition in an amount sufficient to provide the desired physiological effect with no significant, unacceptable toxicity to the subject. The appropriate dosage levels of all biologically active agents, including dopamine receptor agonists, will be readily determined without undue experimentation by the skilled artisan. Because the methods and compositions of the invention provide for enhanced delivery of the dopamine receptor agonists and other active agents, dosage levels significantly lower than conventional dosage levels may be used with success. In general, the active substance will be present in the composition in an amount of from about 0.01% to about 50%, often between about 0.1% to about 20%, and commonly between about 1.0% to 5% or 10% by weight of the total intranasal formulation depending upon the particular substance employed.

Peptide and Protein Agents

The value of biologically active peptides and proteins in medicine has been long recognized in the art. Peptides and proteins are ideal as therapeutics due to their specificity of action, their effectiveness in vivo at relatively low concentrations, and their rapid catalytic activity. For many years, the lack of industrial manufacturing processes for peptides and proteins limited their use as therapeutic agents. However, in recent years the biotechnology and genetic engineering fields have advanced dramatically, making possible the availability of numerous such therapeutic agents for clinical use (see, e.g., Swann, Pharm. Res. 16:826-834, 1998, incorporated herein by reference).

Unfortunately, proteins possess characteristics such as low bioavailability and chemical stability problems (Putney et al., Nature Biotech. 16:153-157, 1998) that may limit their use for treatment of certain diseases. The delivery of peptides and proteins to the body is usually performed by frequent injections. This results in a rapid increase and subsequent rapid decrease of the blood serum concentration levels that could lead to the appearance of side effects. Therefore, the major challenge in this field is to design a system capable of maintaining a blood concentration for a considerable amount of time inside the therapeutic region and to reduce the number of doses that have to be administered.

As used herein, the terms “peptide” and “protein” include polypeptides of various sizes, and do not limit the invention to amino acid polymers of any particular size. Peptides from as small as a few amino acids in length, to proteins of any size, as well as peptide-peptide, protein-protein fusions and protein-peptide fusions, are encompassed by the present invention, so long as the protein or peptide is biologically active in the context of eliciting a specific physiological, immunological, therapeutic, or prophylactic effect or response.

Numerous peptides and proteins have been isolated and developed for use in, for example, treatment of conditions associated with a protein deficiency (e.g., human growth hormone, insulin); enhancement of immune responses (e.g., antibodies, cytokines); treatment of cancer (e.g., cytokines, L-asparaginase, superoxide dismutase, monoclonal antibodies); treatment of conditions associated with excessive or inappropriate enzymatic activity (e.g., inhibition of elastase with alpha-1-antitrypsin, regulation of blood clotting with antithrombin-III); blood replacement therapy (e.g., hemoglobin); treatment of endotoxic shock (e.g., bactericidal-permeability increasing (BPI) protein); and wound healing (e.g., growth factors, erythropoietin). The foregoing examples are only representative of the vast possibilities in the emerging field of peptide and protein therapy.

The formulation and delivery of relatively high molecular weight peptide and protein drugs can present certain problems due to their relatively fragile nature when compared to traditional, smaller molecular weight drugs. In order to successfully employ peptides and proteins as pharmaceuticals, it is essential to understand the many delivery and stability issues relevant to their formulation and effective administration. Peptides and proteins undergo a variety of intra and intermolecular chemical reactions which can lead to their decline or loss of effectiveness as pharmaceuticals. These include oxidation, deamidation, beta-elimination, disulfide scrambling, hydrolysis, isopeptide bond formation, and aggregation. In addition to chemical stability, peptides and proteins must often retain their three dimensional structure in order to maintain their biological activity as therapeutic agents. Loss of the native conformation of peptides and proteins often leads not only to a reduction or loss of biological activity, but also to increased susceptibility to further deleterious processes such as covalent or noncovalent aggregation. Furthermore, the formation of protein aggregates leads to other problems relating to parenteral delivery, such as decreased solubility and increased immunogenicity (see, e.g., H. R. Costantino et al., J. Pharm. Sci., 83:1662-1669, 1994, incorporated herein by reference).

The instant invention provides coordinate administration methods, multi-processing methods, and combinatorial formulations for enhanced mucosal delivery of dopamine receptor agonists and other active agents, including biologically active peptides and proteins. Illustrative examples of therapeutic peptides and proteins for use within this aspect of the invention include, but are not limited to: tissue plasminogen activator (TPA), epidermal growth factor (EGF), fibroblast growth factor (FGF-acidic or basic), platelet derived growth factor (PDGF), transforming growth factor (TGF-alpha or beta), vasoactive intestinal peptide, tumor necrosis factor (TGF), hypothalmic releasing factors, prolactin, thyroid stimulating hormone (TSH), adrenocorticotropic hormone (ACTH), parathyroid hormone (PTH), follicle stimulating hormone (FSF), luteinizing hormone releasing (LHRH), endorphins, glucagon, calcitonin, oxytocin, carbetocin, aldoetecone, enkaphalins, somatostin, somatotropin, somatomedin, gonadotrophin, estrogen, progesterone, testosterone, alpha-melanocyte stimulating hormone, non-naturally occurring opiods, lidocaine, ketoprofen, sufentainil, terbutaline, droperidol, scopolamine, gonadorelin, ciclopirox, olamine, buspirone, calcitonin, cromolyn sodium or midazolam, cyclosporin, lisinopril, captopril, delapril, cimetidine, ranitidine, famotidine, superoxide dismutase, asparaginase, arginase, arginine deaminease, adenosine deaminase ribonuclease, trypsin, chemotrypsin, and papain. Additional examples of useful peptides include, but are not limited to, bombesin, substance P, vasopressin, alpha-globulins, transferrin, fibrinogen, beta-lipoproteins, beta-globulins, prothrombin, ceruloplasmin, alpha2-glycoproteins, alpha₂-globulins, fetuin, alpha₁-lipoproteins, alpha₁-globulins, albumin, prealbumin, and other bioactive proteins and recombinant protein products.

In more detailed aspects of the invention, methods and compositions are provided for enhanced mucosal delivery of specific, biologically active peptide or protein therapeutics in combination with a dopamine receptor agonist to treat (i.e., to eliminate, or reduce the occurrence or severity of symptoms) an existing disease or condition, or to prevent onset of a disease or condition in a subject identified to be at risk therefor. Biologically active peptides and proteins that are useful within these aspects of the invention include, but are not limited to hematopoietics; antiinfective agents; antidementia agents; antiviral agents; antitumoral agents; antipyretics; analgesics; antiinflammatory agents; antiulcer agents; antiallergic agents; antidepressants; psychotropic agents; cardiotonics; antiarrythmic agents; vasodilators; antihypertensive agents such as hypotensive diuretics; antidiabetic agents; anticoagulants; cholesterol lowering agents; therapeutic agents for osteoporosis; hormones; antibiotics; vaccines; and the like.

Biologically active peptides and proteins for use within these aspects of the invention include, but are not limited to, cytokines; peptide hormones; growth factors; factors acting on the cardiovascular system; cell adhesion factors; factors acting on the central and peripheral nervous systems; factors acting on humoral electrolytes and hemal organic substances; factors acting on bone and skeleton growth or physiology; factors acting on the gastrointestinal system; factors acting on the kidney and urinary organs; factors acting on the connective tissue and skin; factors acting on the sense organs; factors acting on the immune system; factors acting on the respiratory system; factors acting on the genital organs; and various enzymes.

For example, hormones which may be administered within the methods and compositions of the present invention include androgens, estrogens, prostaglandins, somatotropins, gonadotropins, interleukins, steroids and cytokines.

Vaccines which may be administered within the methods and compositions of the present invention include bacterial and viral vaccines, such as vaccines for hepatitis, influenza, respiratory syncytial virus (RSV), parainfluenza virus (PIV), tuberculosis, canary pox, chicken pox, measles, mumps, rubella, pneumonia, and human immunodeficiency virus (HIV).

Bacterial toxoids which may be administered within the methods and compositions of the present invention include diphtheria, tetanus, pseudonomas and mycobactrium tuberculosis.

Examples of specific cardiovascular or thromobolytic agents for use within the invention include hirugen, hirulos and hirudine.

Antibody reagents that are usefully administered with the present invention include monoclonal antibodies, polyclonal antibodies, humanized antibodies, antibody fragments, fusions and multimers, and immunoglobins.

Exemplary cytokines for use within the methods and compositions of invention include lymphokines, monokines, hematopoietic factors, and the like, for example interleukins (e.g. interleukin 2 through 11), interleukin-1, tumor necrosis factors (e.g. TNF-alpha and beta), and malignant leukocyte inhibitory factor (LIF), granulocyte colony stimulating factor (G-CSF), granulocyte-macrophage stimulating factor (GM-CSF) and macrophage colony stimulating factor (M-CSF).

Examples of peptide and protein factors which act on bone and skeletal metabolism for use within the methods and compositions of the invention include bone GLa peptide, parathyroid hormone and its active fragments, osteostatin, calcitonin, and histone H4-related bone formation and proliferation peptide.

Exemplary growth factors for use within the methods and compositions of the invention include epidermal growth factor (EGF), fibroblast growth factor (FGF), insulin-like growth factor (IGF), transforming growth factor (TGF), platelet-derived cell growth factor (PDGF), hepatocyte growth factor (HGF), and the like.

Exemplary peptide hormones for use within the methods and compositions of the invention include luteinizing hormone, luteinizing hormone-releasing hormone (LH-RH), adrenocorticotropic hormone (ACTH), amylin, oxytocin, carbetocin, and the like.

With respect to factors acting on the cardiovascular system, exemplary peptides and proteins for use within the methods and compositions of the invention include those which are biologically active to control blood pressure, arteriosclerosis, and other cardiovascular diseases and conditions, exemplified by endothelins, endothelin inhibitors, and endothelin antagonists (see, e.g., EP 436189, EP 457195, EP 496452 and EP 528312, each incorporated herein by reference), endothelin producing enzyme inhibitors, vasopressin, renin, angiotensin I, angiotensin II, angiotensin III, angiotensin I inhibitor, angiotensin II receptor antagonist, antiarrythmic peptide, and so on.

Exemplary peptide and protein factors acting on the central and peripheral nervous systems for use within the methods and compositions of the invention include opioid peptides (e.g. enkepharins, endorphins, kyotorphins), neurotropic factor (NTF), calcitonin gene-related peptide (CGRP), thyroid hormone releasing hormone (TRH), salts and derivatives of TRH (see, e.g., JP Laid Open No. 50-121273/1975; U.S. Pat. No. 3,959,247; JP Laid Open No. 52-116465/1977; U.S. Pat. No. 4,100,152, each incorporated herein by reference), neurotensin, and the like.

Exemplary peptide and protein factors acting on the gastrointestinal system for use within the methods and compositions of the invention include secretin and gastrin.

Exemplary peptide and protein factors acting on humoral electrolytes and hemal organic substances for use within the methods and compositions of the invention include known factors which control hemagglutination, plasma cholesterol level or metal ion concentrations, such as calcitonin, apoprotein E and hirudin Exemplary cell adhesion factors for use within the methods and compositions of the invention include laminin, and intercellular adhesion molecule 1 (ICAM 1).

Exemplary peptide and protein factors acting on the kidney and urinary tract for use within the methods and compositions of the invention include factors which regulate the function of the kidney, such as urotensin.

Exemplary peptide and protein factors acting on the immune system for use within the methods and compositions of the invention include known factors which modulate inflammation and malignant neoplasms, as well as factors which attack infective microorganisms, such as chemotactic peptides and bradykinins.

The biologically active peptides and proteins for use within the invention further include enzymes of natural origin and recombinant enzymes, which include but are not limited to superoxide dismutase (SOD), asparaginase, kallikreins, and the like.

Biologically active peptides and proteins for use within the invention can be peptides or proteins that are readily absorbed into or across the nasal mucosa, but are more typically absorbed poorly (e.g., into the systemic circulation), or not at all, following conventional intranasal delivery/formulation methods. In the latter case, delivery of the peptides or proteins intranasally fails to elicit a therapeutically or prophylactically effective concentration of the peptide or protein at a target compartment (e.g., the systemic circulation) for activity.

Typically, peptides for use within the invention have a molecular weight in the range of about 100 to 200,000, more commonly within the molecular weight range of about 200 to 100,000, and most often within the range of about 200 to 50,000.

Peptide and Protein Analogs and Mimetics

Included within the definition of biologically active peptides and proteins for use within the invention are natural or synthetic, therapeutically or prophylactically active, peptides (comprised of two or more covalently linked amino acids), proteins, peptide or protein fragments, peptide or protein analogs, and chemically modified derivatives or salts of active peptides or proteins. Often, the peptides or proteins are muteins that are readily obtainable by partial substitution, addition, or deletion of amino acids within the naturally occurring peptide or protein sequence. Additionally, fragments of native peptides or proteins are included. Such mutant derivatives and fragments substantially retain the desired biological activity of the native peptide or proteins. In the case of peptides or proteins having carbohydrate chains, biologically active variants marked by alterations in these carbohydrate species are also included. In additional examples, peptides or proteins may be modified by addition or conjugation of a synthetic polymer, such as polyethylene glycol, a natural polymer, such as hyaluronic acid, or an optional sugar (e.g. galactose, mannose), sugar chain, or nonpeptide compound. Substances added to the peptide or protein by such modifications may specify or enhance binding to certain receptors or antibodies. Alternatively, such modifications may render the peptide or protein more lipophilic, e.g., such as may be achieved by addition or conjugation of a phospholipid or fatty acid. Further included within the methods and compositions of the invention are peptides and proteins prepared by linkage (e.g., chemical bonding) of two or more peptides, protein fragments or functional domains (e.g., extracellular, transmembrane and cytoplasmic domains, ligand-binding regions, active site domains, immunogenic epitopes, and the like), for example fusion peptides and proteins recombinantly produced to incorporate the functional elements of a plurality of different peptides or proteins in a single encoded molecule.

Biologically active peptides and proteins for use within the methods and compositions of the invention thus include native or “wild-type” peptides and proteins and naturally occurring variants of these molecules, e.g., naturally occurring allelic variants and mutant proteins. Also included are synthetic, e.g., chemically or recombinantly engineered, peptides and proteins, as well as peptide and protein “analogs” and chemically modified derivatives, fragments, conjugates, and polymers of naturally occurring peptides and proteins. As used herein, the term peptide or protein “analog” is meant to include modified peptides and proteins incorporating one or more amino acid substitutions, insertions, rearrangements or deletions as compared to a native amino acid sequence of a selected peptide or protein, or of a binding domain, fragment, immunogenic epitope, or structural motif, of a selected peptide or protein. Peptide and protein analogs thus modified exhibit substantially conserved biological activity comparable to that of a corresponding native peptide or protein, which means activity (e.g., specific ligand or receptor binding activity) levels of at least 50%, typically at least 75%, often 85%-95% or greater, compared to activity levels of the corresponding native peptide or protein.

For purposes of the present invention, the term biologically active peptide or protein “analog” further includes derivatives or synthetic variants of a native peptide or protein, such as amino and/or carboxyl terminal deletions and fusions, as well as intrasequence insertions, substitutions or deletions of single or multiple amino acids. Insertional amino acid sequence variants are those in which one or more amino acid residues are introduced into a predetermined site in the protein. Random insertion is also possible with suitable screening of the resulting product. Deletional variants are characterized by removal of one or more amino acids from the sequence. Substitutional amino acid variants are those in which at least one residue in the sequence has been removed and a different residue inserted in its place.

Within additional aspects of the invention, peptide mimetics are provided which comprise a peptide or non-peptide molecule that mimics the tertiary binding structure and activity of a selected native peptide or protein functional domain (e.g., binding motif or active site). These peptide mimetics include recombinantly or chemically modified peptides, as well as non-peptide agents such as small molecule drug mimetics, as further described below.

In one aspect, peptides (including polypeptides) useful within the invention are modified to produce peptide mimetics by replacement of one or more naturally occurring side chains of the 20 genetically encoded amino acids (or D amino acids) with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclics. For example, proline analogs can be made in which the ring size of the proline residue is changed from 5 members to 4, 6, or 7 members. Cyclic groups can be saturated or unsaturated, and if unsaturated, can be aromatic or non-aromatic. Heterocyclic groups can contain one or more nitrogen, oxygen, and/or sulphur heteroatoms. Examples of such groups include the furazanyl, furyl, imidazolidinyl, imidazolyl, imidazolinyl, isothiazolyl, isoxazolyl, morpholinyl (e.g. morpholino), oxazolyl, piperazinyl (e.g. 1-piperazinyl), piperidyl (e.g. 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g. 1-pyrrolidinyl), pyrrolinyl, pyrrolyl, thiadiazolyl, thiazolyl, thienyl, thiomorpholinyl (e.g. thiomorpholino), and triazolyl. These heterocyclic groups can be substituted or unsubstituted. Where a group is substituted, the substituent can be alkyl, alkoxy, halogen, oxygen, or substituted or unsubstituted phenyl.

Peptides and proteins, as well as peptide and protein analogs and mimetics, can also be covalently bound to one or more of a variety of nonproteinaceous polymers, e.g., polyethylene glycol, polypropylene glycol, or polyoxyalkenes, in the manner set forth in U.S. Pat. No. 4,640,835; U.S. Pat. No. 4,496,689; U.S. Pat. No. 4,301,144; U.S. Pat. No. 4,670,417; U.S. Pat. No. 4,791,192; or U.S. Pat. No. 4,179,337, all which-are incorporated by reference in their entirety herein.

Other peptide and protein analogs and mimetics within the invention include glycosylation variants, and covalent or aggregate conjugates with other chemical moieties. Covalent derivatives can be prepared by linkage of functionalities to groups which are found in amino acid side chains or at the N- or C-termini, by means which are well known in the art. These derivatives can include, without limitation, aliphatic esters or amides of the carboxyl terminus, or of residues containing carboxyl side chains, O-acyl derivatives of hydroxyl group-containing residues, and N-acyl derivatives of the amino terminal amino acid or amino-group containing residues, e.g., lysine or arginine. Acyl groups are selected from the group of alkyl-moieties including C3 to C18 normal alkyl, thereby forming alkanoyl aroyl species. Covalent attachment to carrier proteins, e.g., immunogenic moieties may also be employed.

In addition to these modifications, glycosylation alterations of biologically active peptides and proteins can be made, e.g., by modifying the glycosylation patterns of a peptide during its synthesis and processing, or in further processing steps. Particularly preferred means for accomplishing this are by exposing the peptide to glycosylating enzymes derived from cells which normally provide such processing, e.g., mammalian glycosylation enzymes. Deglycosylation enzymes can also be successfully employed to yield useful modified peptides and proteins within the invention. Also embraced are versions of a native primary amino acid sequence which have other minor modifications, including phosphorylated amino acid residues, e.g., phosphotyrosine, phosphoserine, or phosphothreonine, or other moieties, including ribosyl groups or cross-linking reagents.

Peptidomimetics may also have amino acid residues that have been chemically modified by phosphorylation, sulfonation, biotinylation, or the addition or removal of other moieties, particularly those which have molecular shapes similar to phosphate groups. In some embodiments, the modifications will be useful labeling reagents, or serve as purification targets, e.g., affinity ligands.

A major group of peptidomimetics within the invention comprises covalent conjugates of native peptides or proteins, or fragments thereof, with other proteins or peptides. These derivatives can be synthesized in recombinant culture such as N- or C-terminal fusions or by the use of agents known in the art for their usefulness in cross-linking proteins through reactive side groups. Preferred peptide and protein derivatization sites for targeting by cross-linking agents are at free amino groups, carbohydrate moieties, and cysteine residues.

Fusion polypeptides between biologically active peptides or proteins and other homologous or heterologous peptides and proteins are also provided. Many growth factors and cytokines are homodimeric entities, and a repeat construct of these molecules or active fragments thereof will yield various advantages, including lessened susceptibility to proteolytic degradation. Various alternative multimeric constructs comprising peptides and proteins useful within the invention are thus provided. In certain embodiments, biologically active polypeptide fusions are provided as described in U.S. Pat. Nos. 6,018,026 and 5,843,725 (each incorporated herein by reference), by linking one or more biologically active peptides or proteins of the invention with a heterologous, multimerizing polypeptide or protein, for example an immunoglobulin heavy chain constant region, or an immunoglobulin light chain constant region. The biologically active, multimerized polypeptide fusion thus constructed can be a hetero- or homo-multimer, e.g., a heterodimer or homodimer, which may each comprise one or more distinct biologically active peptides or proteins operable within the invention. Other heterologous polypeptides may be combined with the active peptide or protein to yield fusions that exhibit a combination of properties or activities of the derivative proteins. Other typical examples are fusions of a reporter polypeptide, e.g., CAT or luciferase, with a peptide or protein of the invention, to facilitate localization of the fused protein (see, e.g., Dull et al., U.S. Pat. No. 4,859,609, incorporated herein by reference). Other gene/protein fusion partners useful in this context include bacterial beta-galactosidase, trpE, Protein A, beta-lactamase, alpha amylase, alcohol dehydrogenase, and yeast alpha mating factor (see, e.g., Godowski et al., Science 241:812-816, 1988, incorporated herein by reference).

The present invention also contemplates the use of biologically active peptides and proteins modified by covalent or aggregative association with chemical moieties, including peptides and proteins bound to or otherwise associated with an active dopamine receptor agonist for therapeutic delivery according to the invention. These derivatives generally fall into the three classes: (1) salts, (2) side chain and terminal residue covalent modifications, and (3) adsorption complexes, for example with cell membranes. The active peptide or protein can also be labeled with a detectable group, for example radioiodinated by the chloramine T procedure, covalently bound to rare earth chelates, or conjugated to another fluorescent moiety for use in diagnostic assays, including assays involving mucosal administration of a coupled or independently labeled dopamine receptor agonist.

Those of skill in the art recognize that a variety of techniques are available for constructing peptide mimetics with the same or similar desired biological activity as the corresponding native peptide compound but with more favorable activity than the peptide with respect to solubility, stability, and/or susceptibility to hydrolysis or proteolysis (see, e.g., Morgan and Gainor, Ann. Rep. Med. Chem. 24:243-252, 1989, incorporated herein by reference). Certain peptidomimetic compounds are based upon the amino acid sequence of the peptides of the invention. Often, peptidomimetic compounds are synthetic compounds having a three-dimensional structure (i.e. a “peptide motif”) based upon the three-dimensional structure of a selected peptide. The peptide motif provides the peptidomimetic compound with the desired biological activity e.g., receptor binding and activation, binding to MHC molecules of one or multiple haplotypes and activating CD8⁺ and/or CD4⁺ T, etc., wherein the subject activity of the mimetic compound is not substantially reduced, and is often the same as or greater than the activity of the native peptide on which the mimetic was modeled. Peptidomimetic compounds can have additional characteristics that enhance their therapeutic application such as increased cell permeability, greater affinity and/or avidity and prolonged biological half-life. The peptidomimetics of the invention typically have a backbone that is partially or completely non-peptide, but with side groups identical to the side groups of the amino acid residues that occur in the peptide on which the peptidomimetic is based. Several types of chemical bonds, e.g. ester, thioester, thioamide, retroamide, reduced carbonyl, dimethylene and ketomethylene bonds, are known in the art to be generally useful substitutes for peptide bonds in the construction of protease-resistant peptidomimetics.

The following describes methods for preparing peptide mimetics modified at the N-terminal amino group, the C-terminal carboxyl group, and/or changing ore or more of the amido linkages in the peptide to a non-amido linkage. It being understood that two or more such modifications can be coupled in one peptide mimetic structure (e.g., modification at the C-terminal carboxyl group and inclusion of a —CH₂-carbamate linkage between two amino acids in the peptide. For N-terminal modifications, peptides typically are synthesized as the free acid but, as noted above, can be readily prepared as the amide or ester. One can also modify the amino and/or carboxy terminus of peptide compounds to produce other compounds useful within the invention. Amino terminus modifications include methylating (i.e., —NHCH₃ or —NH(CH₃)₂), acetylating, adding a carbobenzoyl group, or blocking the amino terminus with any blocking group containing a carboxylate functionality defined by RCOO—, where R is selected from the group consisting of naphthyl, acridinyl, steroidyl, and similar groups. Carboxy terminus modifications include replacing the free acid with a carboxamide group or forming a cyclic lactam at the carboxy terminus to introduce structural constraints. Amino terminus modifications are as recited above and include alkylating, acetylating, adding a carbobenzoyl group, forming a succinimide group, etc. The N-terminal amino group can then be reacted as follows:

(a) to form an amide group of the formula RC(O)NH— where R is as defined above by reaction with an acid halide [e.g., RC(O)Cl] or acid anhydride. Typically, the reaction can be conducted by contacting about equimolar or excess amounts (e.g., about 5 equivalents) of an acid halide to the peptide in an inert diluent (e.g., dichloromethane) preferably containing an excess (e.g., about 10 equivalents) of a tertiary amine, such as diisopropylethylamine, to scavenge the acid generated during reaction. Reaction conditions are otherwise conventional (e.g., room temperature for 30 minutes). Alkylation of the terminal amino to provide for a lower alkyl N-substitution followed by reaction with an acid halide as described above will provide for N-alkyl amide group of the formula RC(O)NR—;

(b) to form a succinimide group by reaction with succinic anhydride. As before, an approximately equimolar amount or an excess of succinic anhydride (e.g., about 5 equivalents) can be employed and the amino group is converted to the succinimide by methods well known in the art including the use of an excess (e.g., ten equivalents) of a tertiary amine such as diisopropylethylamine in a suitable inert solvent (e.g., dichloromethane) (see, for example, Wollenberg, et al., U.S. Pat. No. 4,612,132, incorporated herein by reference). It is understood that the succinic group can be substituted with, for example, C₂-C₆ alkyl or —SR substituents which are prepared in a conventional manner to provide for substituted succinimide at the N-terminus of the peptide. Such alkyl substituents are prepared by reaction of a lower olefin (C₂-C₆) with maleic anhydride in the manner described by Wollenberg, et al. (U.S. Pat. No. 4,612,132) and —SR substituents are prepared by reaction of RSH with maleic anhydride where R is as defined above;

(c) to form a benzyloxycarbonyl —NH— or a substituted benzyloxycarbonyl —NH— group by reaction with approximately an equivalent amount or an excess of CBZ-Cl (i.e., benzyloxycarbonyl chloride) or a substituted CBZ-Cl in a suitable inert diluent (e.g., dichloromethane) preferably containing a tertiary amine to scavenge the acid generated during the reaction;

(d) to form a sulfonamide group by reaction with an equivalent amount or an excess (e.g., 5 equivalents) of R—S(O)₂Cl in a suitable inert diluent (dichloromethane) to convert the terminal amine into a sulfonamide where R is as defined above. Preferably, the inert diluent contains excess tertiary amine (e.g., ten equivalents) such as diisopropylethylamine, to scavenge the acid generated during reaction. Reaction conditions are otherwise conventional (e.g., room temperature for 30 minutes);

(e) to form a carbamate group by reaction with an equivalent amount or an excess (e.g., 5 equivalents) of R—OC(O)Cl or R—OC(O)OC₆H₄-p-NO₂ in a suitable inert diluent (e.g., dichloromethane) to convert the terminal amine into a carbamate where R is as defined above. Preferably, the inert diluent contains an excess (e.g., about 10 equivalents) of a tertiary amine, such as diisopropylethylamine, to scavenge any acid generated during reaction. Reaction conditions are otherwise conventional (e.g., room temperature for 30 minutes);

(f) to form a urea group by reaction with an equivalent amount or an excess (e.g., 5 equivalents) of R—N═C═O in a suitable inert diluent (e.g., dichloromethane) to convert the terminal amine into a urea (i.e., RNHC(O)NH—) group where R is as defined above. Preferably, the inert diluent contains an excess (e.g., about 10 equivalents) of a tertiary amine, such as diisopropylethylamine. Reaction conditions are otherwise conventional (e.g., room temperature for about 30 minutes).

In preparing peptide mimetics wherein the C-terminal carboxyl group is replaced by an ester (i.e., —C(O)OR where R is as defined above), resins as used to prepare peptide acids are typically employed, and the side chain protected peptide is cleaved with base and the appropriate alcohol, e.g., methanol. Side chain protecting groups are then removed in the usual fashion by treatment with hydrogen fluoride to obtain the desired ester.

In preparing peptide mimetics wherein the C-terminal carboxyl group is replaced by the amide —C(O)NR₃R₄, a benzhydrylamine resin is used as the solid support for peptide synthesis. Upon completion of the synthesis, hydrogen fluoride treatment to release the peptide from the support results directly in the free peptide amide (i.e., the C-terminus is —C(O)NH₂). Alternatively, use of the chloromethylated resin during peptide synthesis coupled with reaction with ammonia to cleave the side chain protected peptide from the support yields the free peptide amide and reaction with an alkylamine or a dialkylamine yields a side chain protected alkylamide or dialkylamide (i.e., the C-terminus is —C(O)NRR₁ where R and R₁ are as defined above). Side chain protection is then removed in the usual fashion by treatment with hydrogen fluoride to give the free amides, alkylamides, or dialkylamides.

In another alternative embodiments of the invention, the C-terminal carboxyl group or a C-terminal ester of a biologically active peptide can be induced to cyclize by internal displacement of the —OH or the ester (—OR) of the carboxyl group or ester respectively with the N-terminal amino group to form a cyclic peptide. For example, after synthesis and cleavage to give the peptide acid, the free acid is converted to an activated ester by an appropriate carboxyl group activator such as dicyclohexylcarbodiimide (DCC) in solution, for example, in methylene chloride (CH₂Cl₂), dimethyl formamide (DMF) mixtures. The cyclic peptide is then formed by internal displacement of the activated ester with the N-terminal amine. Internal cyclization as opposed to polymerization can be enhanced by use of very dilute solutions. Such methods are well known in the art.

One can cyclize active peptides for use within the invention, or incorporate a desamino or descarboxy residue at the termini of the peptide, so that there is no terminal amino or carboxyl group, to decrease susceptibility to proteases, or to restrict the conformation of the peptide. C-terminal functional groups among peptide analogs and mimetics of the present invention include amide, amide lower alkyl, amide di(lower alkyl), lower alkoxy, hydroxy, and carboxy, and the lower ester derivatives thereof, and the pharmaceutically acceptable salts thereof.

Other methods for making peptide derivatives and mimetics for use within the methods and compositions of the invention are described in Hruby et al. (Biochem J. 268(2):249-262, 1990, incorporated herein by reference). According to these methods, biologically active peptides serve as structural models for non-peptide mimetic compounds having similar biological activity as the native peptide. Those of skill in the art recognize that a variety of techniques are available for constructing compounds with the same or similar desired biological activity as the lead peptide compound but with more favorable activity than the lead with respect to solubility, stability, and susceptibility to hydrolysis and proteolysis (see, e.g., Morgan and Gainor, Ann. Rep. Med. Chem. 24:243-252, 1989, incorporated herein by reference). These techniques include replacing the peptide backbone with a backbone composed of phosphonates, amidates, carbamates, sulfonamides, secondary amines, and/or N-methylamino acids.

Peptide mimetics wherein one or more of the peptidyl linkages [—C(O)NH—] have been replaced by such linkages as a —CH₂-carbamate linkage, a phosphonate linkage, a —CH₂-sulfonamide linkage, a urea linkage, a secondary amine (—CH₂NH—) linkage, and an alkylated peptidyl linkage [—C(O)NR₆— where R₆ is lower alkyl] are prepared during conventional peptide synthesis by merely substituting a suitably protected amino acid analogue for the amino acid reagent at the appropriate point during synthesis. Suitable reagents include, for example, amino acid analogues wherein the carboxyl group of the amino acid has been replaced with a moiety suitable for forming one of the above linkages. For example, if one desires to replace a—C(O)NR— linkage in the peptide with a —CH₂-carbamate linkage (—CH₂OC(O)NR—), then the carboxyl (—COOH) group of a suitably protected amino acid is first reduced to the —CH₂OH group which is then converted by conventional methods to a —OC(O)Cl functionality or a para-nitrocarbonate —OC(O)O—C₆H₄-p-NO₂ functionality. Reaction of either of such functional groups with the free amine or an alkylated amine on the N-terminus of the partially fabricated peptide found on the solid support leads to the formation of a—CH₂OC(O)NR— linkage. For a more detailed description of the formation of such —CH₂-carbamate linkages, see, e.g., Cho et al. (Science 261:1303-1305, 1993, incorporated herein by reference).

Replacement of an amido linkage in an active peptide with a —CH₂-sulfonamide linkage can be achieved by reducing the carboxyl (—COOH) group of a suitably protected amino acid to the —CH₂OH group, and the hydroxyl group is then converted to a suitable leaving group such as a tosyl group by conventional methods. Reaction of the derivative with, for example, thioacetic acid followed by hydrolysis and oxidative chlorination will provide for the —CH₂—S(O)₂Cl functional group which replaces the carboxyl group of the otherwise suitably protected amino acid. Use of this suitably protected amino acid analogue in peptide synthesis provides for inclusion of an —CH₂S(O)₂NR— linkage which replaces the amido linkage in the peptide thereby providing a peptide mimetic. For a more complete description on the conversion of the carboxyl group of the amino acid to a —CH₂S(O)₂Cl group, see, e.g., Weinstein and Boris (Chemistry & Biochemistry of Amino Acids, Peptides and Proteins, Vol. 7, pp. 267-357, Marcel Dekker, Inc., New York, 1983, incorporated herein by reference). Replacement of an amido linkage in an active peptide with a urea linkage can be achieved in the manner set forth in U.S. patent application Ser. No. 08/147,805 (incorporated herein by reference).

Secondary amine linkages wherein a —CH₂NH— linkage replaces the amido linkage in the peptide can be prepared by employing, for example, a suitably protected dipeptide analogue wherein the carbonyl bond of the amido linkage has been reduced to a CH₂ group by conventional methods. For example, in the case of diglycine, reduction of the amide to the amine will yield after deprotection H₂NCH₂CH₂NHCH₂ COOH that is then used in N-protected form in the next coupling reaction. The preparation of such analogues by reduction of the carbonyl group of the amido linkage in the dipeptide is well known in the art.

The biologically active peptide and protein agents of the present invention may exist in a monomeric form with no disulfide bond formed with the thiol groups of the cysteine residue(s). Alternatively, an intermolecular disulfide bond between the thiol groups of cysteines on two or more peptides or proteins can be produced to yield a multimeric (e.g., dimeric, tetrameric or higher oligomeric) compound. Certain of such peptides and proteins can be cyclized or dimerized via displacement of the leaving group by the sulfur of a cysteine or homocysteine residue (see, e.g., Barker et al., J. Med. Chem. 35:2040-2048, 1992; and Or et al., J. Org. Chem. 56:3146-3149, 1991, each incorporated herein by reference). Thus, one or more native cysteine residues may be substituted with a homocysteine. Intramolecular or intermolecular disulfide derivatives of active peptides and proteins provide analogs in which one of the sulfurs has been replaced by a CH₂ group or other isostere for sulfur. These analogs can be made via an intramolecular or intermolecular displacement, using methods known in the art as shown below. One of skill in the art will readily appreciate that this displacement can also occur using other homologs of the a-amino-g-butyric acid derivative shown above and homocysteine.

All of the naturally occurring, recombinant, and synthetic peptides and proteins and peptide and protein analogs and mimetics identified as useful agents within the invention can be used for screening (e.g., in kits and/or screening assay methods) to identify additional compounds, including other peptides, proteins, analogs and mimetics, that will function within the methods and compositions of the invention. Several methods of automating assays have been developed in recent years so as to permit screening of tens of thousands of compounds in a short period (see, e.g., Fodor et al., Science 251:767-773, 1991, and U.S. Pat. Nos. 5,677,195; 5,885,837; 5,902,723; 6,027,880; 6,040,193; and 6,124,102, issued to Fodor et al., each incorporated herein by reference). Large combinatorial libraries of compounds can be constructed by encoded synthetic libraries (ESL) described in, e.g., WO 95/12608, WO 93/06121, WO 94/08051, WO 95/35503, and WO 95/30642 (each incorporated by reference). Peptide libraries can also be generated by phage display methods (see, e.g., Devlin, WO 91/18980, incorporated herein by reference). Many other publications describing chemical diversity libraries and screening methods are also considered reflective of the state of the art pertaining to these aspects of the invention and are generally incorporated herein.

One method of screening for new biologically active agents for use within the invention (e.g., small molecule drug peptide mimetics) utilizes eukaryotic or prokaryotic host cells which are stably transformed with recombinant DNA molecules expressing an active peptide or protein. Such cells, either in viable or fixed form, can be used for standard assays, e.g., ligand/receptor binding assays (see, e.g., Parce et al., Science 246:243-247, 1989; and Owicki et al., Proc. Natl. Acad. Sci. USA 87:4007-4011, 1990, each incorporated herein by reference). Competitive assays are particularly useful, for example assays where the cells are contacted and incubated with a labeled receptor or antibody having known binding affinity to the peptide ligand, and a test compound or sample whose binding affinity is being measured. The bound and free labeled binding components are then separated to assess the degree of ligand binding. The amount of test compound bound is inversely proportional to the amount of labeled receptor binding to the known source. Any one of numerous techniques can be used to separate bound from free ligand to assess the degree of ligand binding. This separation step can involve a conventional procedure such as adhesion to filters followed by washing, adhesion to plastic followed by washing, or centrifugation of the cell membranes.

Another technique for drug screening within the invention involves an approach which provides high throughput screening for compounds having suitable binding affinity to a target molecule, e.g., a chemokine receptor, and is described in detail in Geysen, European Patent Application 84/03564, published on Sep. 13, 1984. First, large numbers of different test compounds, e.g., small peptides, are synthesized on a solid substrate, e.g., plastic pins or some other appropriate surface, (see, e.g., Fodor et al., Science 251:767-773, 1991, and U.S. Pat. Nos. 5,677,195; 5,885,837; 5,902,723; 6,027,880; 6,040,193; and 6,124,102, issued to Fodor et al., each incorporated herein by reference). Then all of the pins are reacted with a solubilized peptide agent of the invention, and washed. The next step involves detecting bound peptide.

Rational drug design may also be based upon structural studies of the molecular shapes of biologically active peptides and proteins determined to operate within the methods of the invention. Various methods are available and well known in the art for characterizing, mapping, translating, and reproducing structural features of peptides and proteins to guide the production and selection of new peptide mimetics, including for example x-ray crystallography and 2 dimensional NMR techniques. These and other methods, for example, will allow reasoned prediction of which amino acid residues present in a selected peptide or protein form molecular contact regions necessary for specificity and activity (see, e.g., Blundell and Johnson, Protein Crystallography, Academic Press, N.Y., 1976, incorporated herein by reference).

Aggregation Inhibitory Agents and Methods

Protein aggregation is of major importance in biotechnology for the in vitro production and in vivo use of recombinant peptides proteins. Aggregation commonly limits the stability, solubility and yields of recombinant proteins for use in pharmaceutical formulations. Further, in vivo protein aggregation or precipitation is the cause, or an associated pathological symptom, in amyloid diseases such as Down's syndrome, Alzheimer's disease, diabetes and/or cataracts, as well as in other disorders. In this context, several peptides, including beta-amyloid peptides, have been shown to spontaneously self-associate, or aggregate, into linear, unbranched fibrils in serum or in isotonic saline. At least fifteen different polypeptides are known to be capable of causing in vivo different forms of amyloidosis via their deposition in particular organs or tissues as insoluble protein fibrils.

Under various conditions, therapeutic peptides and proteins for use within the invention may exhibit functionally deleterious aggregation. Commonly, peptides and proteins expressed in large quantities in heterologous expression systems precipitate within the recombinant host cell in dense aggregates. Such insoluble aggregates of expressed polypeptide (inclusion bodies) may reflect improperly folded polypeptides relating to the inability of the host cell to properly process and/or secrete the recombinant polypeptide. The aggregated fraction often constitutes a major fraction of total cell protein in recombinant expression systems. Further details of peptide and protein aggregation are provided in Brems et al., Biochemistry, 24:7662, 1985; Mitraki et al., Bio/Technology, 7:690, 1989; Marston and Hartley, Methods in Enzymol., 182:264-276 (1990); Wetzel, “Protein Aggregation In vivo: Bacterial Inclusion Bodies and Mammalian Amyloid,” in Stability of Protein Pharmaceuticals: In vivo Pathways of Degradation and Strategies for Protein Stabilization, Ahern and Manning (eds.) (Plenum Press, 1991); and Wetzel, “Enhanced Folding and Stabilization of Proteins by Suppression of Aggregation In vitro and In vivo,” in Protein Engineering—A Practical Approach, Rees, A. R. et al. (eds.) (IRL Press at Oxford University Press, Oxford, 1991) (each of the foregoing publications is incorporated herein by reference).

Recovery of therapeutic peptides and proteins from aggregate forms, e.g., as found in recombinant expression systems, presents numerous problems. In many cases, peptides and proteins recovered from aggregates are predominantly biologically inactive, often because they folded into a three-dimensional conformation different from that of native protein. Misfolding can occur either in the cell during fermentation or during protein isolation, processing or storage procedures. Methods for preventing aggregation, and for isolating and refolding proteins from aggregated complexes into a correct, biologically active conformation, are therefore important for obtaining functional proteins for therapeutic use within the invention.

Accordingly, the present invention provides methods for mucosal delivery of dopamine receptor agonists that are effective in producing or maintaining “unaggregated” peptides or proteins in a mucosal delivery formulation. The methods involve solubilizing peptides and proteins from aggregates and/or stabilizing peptides and proteins that are prone to aggregation—to provide formulations of soluble, stable, biologically active peptide or protein suitable for mucosal administration. The peptide or protein thus stabilized in soluble form may be bound or otherwise associated (e.g., as a carrier) with the dopamine receptor agonist, or may be admixed or otherwise coordinately administered therewith as an adjunct therapeutic or mucosal delivery-enhancing agent (e.g., a degradative enzyme inhibitor). Such formulations contain the solubilized peptide or protein in a substantially pure, unaggregated and therapeutically useful form.

Typically, the peptide or protein which is solubilized from aggregate or stabilized to reduce aggregation is initially obtained from a recombinant expression system, often from insoluble aggregate form. The latter procedure typically involves disruption of the host cells and separation of the ruptured cell materials from the insolubilized protein (as inclusion bodies). Examples of available means for accomplishing this are procedures involving the use of sonication and homogenization in the presence of one or more detergents and separation of the ruptured cell materials from the aggregated peptide or protein by centrifugation (see, e.g., U.S. Pat. Nos. 4,828,929 and 4,673,641). It should be understood that other well known procedures can be also be used in this context.

Peptides or proteins recovered from recombinant systems in this manner typically comprise a broad spectrum of polypeptides ranging from soluble monomers and multimers to macroscopic insoluble structures in which thousands of such individual polypeptide fragments are bound. Typically, however, those aggregates composed of approximately 10 to 20, or fewer fragments, and having a molecular weight of 200,000 to 400,000 are soluble. Such fragments, which are referred to herein as “soluble aggregate”, have relatively low therapeutic utility as measured in in vitro assays. Certain even larger complexes are also soluble, although also of relatively low therapeutic utility.

As used herein, “unaggregated” peptide or protein comprise peptide or protein that is substantially free of aggregate, whether soluble or insoluble. The composition of unaggregated peptide or protein typically comprises a population of monomeric peptide or protein, but may also include noncovalently linked multimeric species. Typically, the amount of “soluble aggregate” present in such samples (e.g., as determined by high performance liquid chromatography (HPLC)) is less than about 15%, often less than about 5%, and commonly less than about 0.5% of the subject peptide or protein species in a preparation. In alternate terms, the compositions of the invention are “substantially free of aggregate”, wherein the percent by weight of monomer in a purified peptide or protein preparation is at least about 40% to 65%, more typically about 65% to 80 weight %, often at least 75%-95% or greater.

For some peptides and proteins, the formation of inclusion bodies and other types of insoluble aggregates may be related to the presence of cysteine residues in the subject peptide or protein. It is believed that incorrect disulfide bonds are encouraged to form either within inclusion bodies or during attempts to solubilize the polypeptides therefrom, as well as under other purification or storage conditions. When such bonds are formed within a polypeptide (an intrachain bond), they may lead to a biologically inactive conformation of the molecule. When disulfide bonds are formed between fragments (an interchain bond), they may lead to insoluble or biologically inactive dimers or aggregates. Illustrative of this phenomenon, misfolded IGF-I possesses different disulfide bond pairs than are found in native IGF-I, and exhibits significantly reduced biological activity (Raschdorf et al., Biomedical and Environmental Mass Spectroscopy, 16:3-8, 1988, incorporated herein by reference). In other cases, proteins isolated from aggregates produce disulfide-linked dimers, trimers, and multimers (Morris et al., Biochem. J., 268:803-806, 1990; Toren et al., Anal. Biochem., 169:287-299, 1988; Frank et al., in Peptides: synthesis-structure-function,” ed. D. H. Rich and E. Gross, pp. 729-738 (Pierce Chemical Company: Rockford, Ill., 1981), each incorporated herein by reference). This association phenomenon is very common during protein refolding, particularly at higher protein concentrations, and appears to often involve association through hydrophobic interaction of partially folded intermediates (Cleland and Wang, Biochemistry, 29:11072-11078, 1990, incorporated herein by reference).

Thus, successful manipulation of mammalian proteins expressed from recombinant bacterial systems has generally required that the cysteine residues thereof be altered so that they cannot react with other cysteine residues. Without this treatment, undesired reaction of the cysteine residues thereof typically occurs, leading to the formation of insoluble or biologically inactive polypeptide aggregates unsuited for effective use as therapeutics.

There are numerous well known procedures which can be used within the invention to successfully alter cysteine residues of therapeutic or delivery-enhancing peptides and proteins that are prone to aggregation involving disulfide bonding. One such technique involves treatment of cysteine residues with a reducing agent such as; for example, beta-mercaptoethanol or dithiothreitol (DTT) followed by permanent alkylation (for example, with iodoacetamide) of the cysteine residues. Numerous other covalent labels may be attached to the target cysteine residues, so long as they are applied under pH conditions that do not irreversibly denature the target peptide or protein and do not allow chemical reaction with other cysteine residues. Such covalent labeling procedures are generally known in the art and include also, for example, reaction with iodoacetic acid or iodinating agents such as iodofluorescein. Additionally, cysteine residues may be chemically altered such as by sulfitolyzation. Alteration can be accomplished also by site directed mutagenesis of an encoding DNA, replacing cysteine residues with “inert” residues such as, for example, glycine or alanine, or by deletion of sequence positions corresponding to cysteine. A sufficient number of the cysteine residues are altered to avoid the aggregation problems caused by their presence. For additional details regarding methods for preparing cysteine-altered proteins to minimize aggregation, see, e.g., U.S. Pat. No. 5,847,086 (incorporated herein by reference).

For methods that do not involve cysteine modification, it is important to note that protein folding is influenced by the nature of the medium containing the protein, and by a combination of weak attractive or repellent intramolecular forces involved in hydrogen bonding, ionic bonding, and hydrophobic interactions. When pairs of cysteine residues are brought into close proximity as the peptide backbone folds, strong covalent disulfide bonds often form between cysteine residues, serving to lock the tertiary conformation in place. Refolding protocols have been designed to break incorrect disulfide bonds, block random disulfide bonding, and allow refolding and correct disulfide bonding under conditions favorable to the formation of an active conformer.

One general method for recovering active protein from aggregates involves solubilizing the aggregated protein in strongly denaturing solutions and then optionally exchanging weakly denaturing solutions for the strongly denaturing solutions (or diluting the strong denaturant), or using molecular sieve or high-speed centrifugation techniques (see, e.g., U.S. Pat. Nos. 4,512,922; 4,518,256; 4,511,502; and 4,511,503, incorporated herein by reference). Such recovery methods are useful within certain multi-processing methods of the invention to prepare active peptide and protein compositions from aggregated, or aggregation-prone, starting materials. The terms “denaturant” are broadly applied herein to include denaturant and detergent compounds that unfold proteins and/or disrupt disulfide bonds and other interactions between aggregate-prone peptides and proteins. Examples of suitable materials for use as denaturants in this context include, but are not limited to, the denaturants urea and guanidine-hydrochloride, and detergents such as polyoxyethylene p-tert-octylphenol (Nonidet®P40), polyoxyethylene, p-tert-octylphenol (Triton-X-100), and sodium deoxycholate. Often, the formulations and methods of the invention will incorporate urea as the selected denaturant, because it is highly soluble in aqueous solutions and it is capable of being removed rapidly from solution by dialysis. In addition, because urea is a nonionic substance, it does not interfere with ion exchange materials that may be used in the process to remove contaminants of bacterial origin such as DNA and endotoxin. Although numerous procedures are known for solubilizing aggregated inclusion body proteins in the presence of denaturant, clinical use of the resultant product requires that the denaturant contained therein be replaced with clinically acceptable materials which are nontoxic and nonirritating, so that the resultant solution complies with medical standards for injection into humans.

Certain aggregation inhibitory methods for use within the invention seek to eliminate random disulfide bonding prior to coaxing the recombinant protein into its biologically active conformation. The denatured peptide or protein to be refolded is then further purified under reducing conditions that maintain the cysteine moieties of the protein as free sulfhydryl groups. The reducing agent is then diluted into an aqueous solution to enable the refolded protein to form the appropriate disulfide bonds in the presence of air or some other oxidizing agent. This enables refolding to be easily incorporated into the overall purification or formulation process.

In another approach that is useful within the methods of the invention, refolding of recombinant peptide or protein takes place in the presence of both the reduced (R—SH) and oxidized (R—S—S—R) forms of a sulfhydryl compound. This allows free sulfhydryl groups and disulfides to be formed and reformed constantly throughout the purification process. The reduced and oxidized forms of the sulfhydryl compound are provided in a buffer having sufficient denaturing power that all of the intermediate conformations of the protein remain soluble in the course of the unfolding and refolding. Urea is a suitable buffer medium because of its apparent ability to act both as a sufficiently weak denaturing agent to allow the protein to approximate its correct conformation, and as a sufficiently strong denaturant that the refolding intermediates maintain their solubility.

Yet another alternative purification/preparative technique for use within the mucosal delivery methods of the invention is designed to break any disulfide bonds that may have formed incorrectly during isolation of peptide or protein from aggregated form, and then to derivatize the available free sulfhydryl groups of the recombinant protein. This objective is achieved by sulfonating the protein to block random disulfide pairings, allowing the protein to refold correctly in weak denaturant, and then desulfonating the protein, under conditions that favor correct disulfide bonding. The desulfonation takes place in the presence of a sulfhydryl compound and a small amount of its corresponding oxidized form to ensure that suitable disulfide bonds will remain intact. The pH is raised to a value such that the sulfhydryl compound is at least partially in ionized form to enhance nucleophilic displacement of the sulfonate.

Additional recovery methods useful for isolating active peptide and protein from aggregated form for intranasal administration according to the invention is provided in WO 88/8003, and Halenbeck et al., Bio/Technology, 7:710-715, 1989 (each incorporated herein by reference). These procedures involve initial solubilization of monomers isolated from inclusion bodies under reducing conditions in a chaotropic environment comprising urea or guanidine hydrochloride, followed by refolding by stepwise dilution of the chaotropic agents, and final oxidation of the refolded molecules in the presence of air or a redox-system.

It is also contemplated that certain aggregated peptides and proteins to be formulated and/or mucosally administered with a dopamine receptor agonist according to the methods of the invention will be solubilized and sulphitolysed in denaturant, then precipitated by solvent exchange (see, e.g., U.S. Pat. No. 4,923,967; and EP 361,830, each incorporated herein by reference). According to this technique, the precipitated protein is resolubilized in denaturant and allowed to refold in the presence of reducing agent.

Additional methods useful within the invention for refolding proteins to an active form for intranasal administration involve the use of high concentrations of copper as an oxidant, as employed for interleukin-2 (IL-2) (Tsuji et al., Biochemistry, 26:3129-3134, 1987; WO 88/8849, each incorporated herein by reference). According to another technique, a denaturing agent and reducing agent are added to solubilize the protein, followed by removal of the reducing agent, oxidation of the protein, and removal of the denaturant, as employed for growth hormone (U.S. Pat. No. 4,985,544, each incorporated herein by reference). Other methods for refolding growth hormone are disclosed in George et al., DNA, 4:273-281, 1984; Gill et al., Bio/Technology, 3:643-646, 1985; Sekine et al., Proc. Natl. Acad. Sci. USA, 82:4306-4310, 1985 (each incorporated herein by reference). Yet additional refolding methods useful within the invention are described in Green et al., J. Dairy Res., 52:281-286, 1985; Winkler et al., Bio/Technology, 3:990-1000, 1985; U.S. Pat. No. 4,652,630 (urea used for solubilization, followed by a mild oxidizing agent for refolding); EP 360,937; Boss et al., Nucl. Acids Res., 12:3791-3806, 1984; Cabilly et al., Proc. Natl. Acad. Sci. USA, 81:3273-3277, 1984,; Marston et al., Bio/Technology, 2:800-804, 1984; and Marston, Biochem. J., 240:1-12, 1986 (each incorporated herein by reference).

Yet additional techniques for refolding peptides and proteins to forms more suitable for mucosal administration and/or formulation with a dopamine receptor agonist involve the use of SDS for solubilization and Cu+² ions as oxidation promoters of the fully reduced proteins (e.g., as exemplified for IL-2 and IFN-Beta in U.S. Pat. No. 4,572,798, incorporated herein by reference). Alternative methods for preparing active recombinant proteins from aggregates are described in U.S. Pat. No. 4,620,948 (incorporated herein by reference), which involve using strong denaturing agents to solubilize the proteins, reducing conditions to facilitate correct folding, and denaturant replacement in the presence of air or other oxidizing agents to reform the disulfide bonds.

Alternate methods for renaturing unfolded peptides and proteins within the compositions and methods of the invention involve reversibly binding the denatured peptide or protein to a solid matrix and stepwise renaturing it by diluting the denaturant (as exemplified for cytochrome c, ovalbumin, and trypsin inhibitor in WO 86/5809, incorporated herein by reference). Alternatively, peptides and proteins from aggregates can be S-sulfonated during purification to protect thiol moieties and then dimerized in the presence of oxidizing agents to yield an active product (as described for a modified monomeric form of human platelet-derived growth factor (PDGF) expressed in E. coli by Hoppe et al., Biochemistry, 28:2956-2960, 1989, incorporated herein by reference).

Additionally, EP 433,225, published Jun. 19, 1991, discloses a process for producing dimeric biologically active transforming growth factor-β protein or a salt thereof wherein the denatured monomeric form of the protein is subjected to refolding conditions that include a solubilizing agent such as mild detergent, an organic, water-miscible solvent, and/or a phospholipid. U.S. Pat. No. 4,705,848 discloses the isolation of monomeric, biologically active growth hormone from inclusion bodies using one denaturing step with a guanidine salt and one renaturing step. Bowden et al., Bio/Technology, 9:725-730, 1991; Samuelsson et al., Bio/Technology, 9:731, 1991; and Hejnaes et al., Protein Engineering, 5:797-806, 1992 (each incorporated herein by reference) describe additional procedures and reagents that are useful to prepare and/or stabilize aggregation-prone peptides and proteins within the multiprocessing methods of the invention.

Other methods useful within the invention for resolving aggregation problems involve disulfide exchange equilibration of refolding intermediates. For example, the refolding of IGF-I using redox buffers was investigated and the partially oxidized IGF-I forms produced were characterized by Hober et al., Biochemistry, 31:1749-1756, 1992. Disulfide exchange can also be modulated using the additive agent of peptidyl disulfide isomerase (PDI) or peptidyl prolyl isomerase (PPI). See, for example, JP Pat. Appln. No. 63294796; EP 413,440; and EP 293,793, each incorporated herein by reference).

Enhancement of selected disulfide pairings, e.g., by adding 50% methanol to buffer at low ionic strength, is another useful method for preparing active peptide and protein reagents for intranasal administration according to the invention (see, e.g., Snyder, J. Biol. Chem., 259:7468-7472, 1984, incorporated herein by reference). This method involves enhancing formation of specific disulfide bonds by adjusting electrostatic factors in the medium to favor the juxtaposition of oppositely charged amino acids that border the selected cysteine residues (see also, Tamura et al., abstract and poster presented at the Eleventh American Peptide Symposium on Jul. 11, 1989, incorporated herein by reference, which discloses addition of acetonitrile, DMSO, methanol, or ethanol to improve processing of correctly folded IGF-1).

Related methods that are useful within the invention involve changing the redox potential of a subject peptide or protein by dialysis against a buffer containing from 20-40% v/v ethanol over a period of up to five hours and acidifying the mixture, e.g., as disclosed for AlaGlu-IGF-I in WO 92/03477 (incorporated herein by reference). Alternatively, methanol can be used at certain concentrations in the denaturation of active peptides and proteins (Lustig et al., Biochim. Biophys. Acta, 1119:205-210, 1992 (incorporated herein by reference). Yet additional methods involve the use of moderate concentrations of alcohol or other methods of modulating solution polarity to reduce association of peptides under conditions that promote structure destabilization (Bryant et al., Biochemistry, 31:5692-5698, 1992; Huaet al., Biochim. Biophys. Acta, 1078:101-110, 1991; Brems et al., Biochemistry, 29:9289-9293, 1990; JP 62-190199, Jackson et al., Biochim Biophys. Acta, 1118:139-143, 1992; Shibata et al., Biochemistry, 31:5728-5733, 1992; Zhong et al., Proc. Natl. Acad. Sci. USA, 89:4462-4465, 1992, each incorporated herein by reference).

In additional methods useful within the invention, low copper or manganese concentrations are used to facilitate disulfide oxidation of polypeptides (see, e.g., U.S. Pat. No. 5,756,672, incorporated herein by reference). The peptide or protein is first maintained in an alkaline buffer comprising a chaotropic agent and a reducing agent in amounts sufficient for solubilization. During the refolding or processing step the polypeptide is incubated at a concentration of about 0.1 to 15 mg/mL in a buffer of pH 7-12 comprising about 5-40% (v/v) of an alcoholic or polar aprotic solvent, about 0.2 to 3M of an alkaline earth, alkali metal, or ammonium salt, about 0.1 to 9M of a chaotropic agent, and about 0.01 to 15 μM of a copper or manganese salt. An oxygen source is introduced, so that refolding of the peptide or protein occurs during the incubation. The essence of this method involves the use of a special buffer containing a minimal concentration of copper or manganese salt to enhance refolding of misfolded polypeptides. The use of manganese or copper salts as oxidation catalysts avoids the necessity of more expensive disulfide-exchange agents such as glutathione. Furthermore, the method avoids the possibility of producing polypeptide containing disulfide adducts that can result when disulfide-exchange agents are employed.

Additional techniques useful within the methods and compositions of the invention involve the use of a pro-sequence of a naturally occurring polypeptide to promote folding of a biologically inactive polypeptide to its active form (e.g., as exemplified for subtilisin in U.S. Pat. No. 5,191,063, incorporated herein by reference).

The foregoing recovery, purification and preparative methods and compositions are generally useful to prepare formulations of aggregation-prone peptides and proteins for formulation and/or coordinate, mucosal administration with a dopamine receptor agonist. These methods and compositions of the invention further reduce aggregation problems that occur during storage, delivery, and even after delivery when pharmaceutical formulations comprising aggregation-prone biologically active agents or carriers are delivered to, or absorbed into or across, a mucosal surface. By determining the molecular pathways that contribute to aggregation of solid peptides and proteins, rational approaches for stabilization in accordance with the foregoing teachings are readily determined. These approaches specifically target the particular mechanisms involved in aggregation of a selected biologically active peptide or protein within the invention. In conjunction with these strategies, the methods and compositions of the invention, e.g., which involve admixtures or complexes of peptides or proteins with a dopamine receptor agonist or other mucosal formulation component (e.g., a polymeric matrix or delivery vehicle), maintain the level of moisture activity within the formulation at optimal levels to reduce peptide or protein aggregation. This can be achieved, for example, selecting a carrier or delivery vehicle that provides for reduced water activities. The pH of the microenvironment for storage and/or delivery is also controlled to minimize peptide or protein aggregation, following the application of physicochemical principles set forth herein.

Another approach for stabilizing solid protein formulations of the invention is to increase the physical stability of purified, e.g., lyophilized, protein components of a preparation for mucosal delivery. This will inhibit aggregation via hydrophobic interactions as well as via covalent pathways which may increase as proteins unfold. Stabilizing formulations in this context often include polymer based formulations, for example a biodegradable hydrogel formulation/delivery system. As noted above, the critical role of water in protein structure, function, and stability is well known. Typically, proteins are relatively stable in the solid state with bulk water removed. However, solid therapeutic protein formulations may become hydrated upon storage at elevated humidities or during delivery from a sustained release device. The stability of proteins generally drops with increasing hydration. Water can also play a significant role in solid protein aggregation, for example, by increasing protein flexibility resulting in enhanced accessibility of reactive groups, by providing a mobile phase for reactants, and by serving as a reactant in several deleterious processes such as beta-elimination and hydrolysis.

Protein preparations containing between about 6% to 28% water are the most unstable. Below this level, the mobility of bound water and protein internal motions are low. Above this level, water mobility and protein motions approach those of full hydration. Up to a point, increased susceptibility toward solid-phase aggregation with increasing hydration has been observed in several systems. However, at higher water content, less aggregation is observed because of the dilution effect.

In accordance with these principles, an effective method for stabilize peptides and proteins against solid-state aggregation for formulation or coordinate administration with a dopamine receptor agonist is to control the water content in a solid formulation and maintain the water activity in the formulation at optimal levels. This level depends on the nature of the protein, but in general, proteins maintained below their “monolayer” water coverage will exhibit superior solid-state stability. According to current FDA requirements, an acceptable protein drug containing pharmaceutical product should exhibit less than 10% deterioration after 2 years (Cleland, J. L. and Langer, R. In formulation and delivery of proteins and peptides, ACS books, 1994, incorporated herein by reference).

A variety of additives, diluents, bases and delivery vehicles are provided within the invention that effectively control water content to enhance protein stability. These reagents and carrier materials effective as anti-aggregation agents in this sense include, for example, polymers of various functionalities, such as polyethylene glycol, dextran, diethylaminoethyl dextran, and carboxymethyl cellulose, which significantly increase the stability and reduce the solid-phase aggregation of peptides and proteins admixed therewith or linked thereto. In some instances, the functionality or physical stability of proteins can also be increased by various additives to aqueous solutions of the peptide or protein drugs. Additives, such as polyols (including sugars), amino acids, proteins such as collagen and gelatin and certain salts may be used.

Certain additives, in particular sugars and other polyols, also impart significant physical stability to dry, e.g., lyophilized proteins. These additives can also be used within the invention to protect the proteins against aggregation not only during lyophilization but also during storage in the dry state. For example sucrose and Ficoll 70 (a polymer with sucrose units) exhibit significant protection against peptide or protein aggregation during solid-phase incubation under various conditions. These additives may also enhance the stability of solid proteins embedded within polymer matrices.

Yet additional additives, for example sucrose, stabilize proteins against solid-state aggregation in humid atmospheres at elevated temperatures, as will occur in many sustained release formulations of the invention. Proteins such as gelatin and collagen also serve as stabilizing or bulking agents to reduce denaturation and aggregation of unstable proteins in this context. These additives can be incorporated into polymeric melt processes and compositions within the invention. For example, polypeptides microparticles can be prepared by simply lyophilizing or spray drying a solution containing various stabilizing additives described above. Sustained release of unaggregated peptides and proteins can thereby be obtained over an extended period of time.

Various additional preparative components and methods, as well as specific formulation additives, are provided herein which yield formulations or coordinate administration methods for mucosal delivery of dopamine receptor agonist in combination with aggregation-prone peptides and proteins, wherein the peptide or protein is stabilized in a substantially pure, unaggregated form. A range of components and additives are contemplated for use within these methods and formulations. Exemplary of these anti-aggregation agents are linked dimers of cyclodextrins (CDs), which selectively bind hydrophobic side chains of polypeptides (see, e.g., Breslow, et al., J. Am. Chem. Soc. 120:3536-3537; Maletic, et al., Angew. Chem. Int. Ed. Engl. 35:1490-1492; each incorporated herein by reference). These CD dimers have been found to bind to hydrophobic patches of proteins in a manner that significantly inhibits aggregation (Leung et al., Proc. Nat.l Acad. Sci. USA 97:5050-5053, 2000, incorporated herein by reference). This inhibition is selective with respect to both the CD dimer and the protein involved. Such selective inhibition of protein aggregation provides additional advantages within the mucosal delivery methods and compositions of the invention. Additional agents for use in this context include CD trimers and tetramers with varying geometries controlled by the linkers that specifically block aggregation of peptides and proteins (Breslow et al., J. Am. Chem. Soc. 118:11678-11681, 1996; Breslow et al., PNAS USA 94:11156-11158, 1997; Breslow et al., Tetrahedron Lett. 2887-2890, 1998, each incorporated herein by reference).

Yet additional anti-aggregation agents and methods for incorporation within the invention involve the use of peptides and peptide mimetics to selectively block protein-protein interactions. In one aspect, the specific binding of hydrophobic side chains reported for CD multimers is extended to proteins via the use of peptides and peptide mimetics that similarly block protein aggregation. A wide range of suitable methods and anti-aggregation agents are available for incorporation within the compositions and procedures of the invention (Zutshi et al., Curr. Opin. Chem. Biol. 2:62-66, 1998; Daugherty et al., J. Am. Chem. Soc. 121:4325-4333, 1999: Zutshi et al., J. Am. Chem. Soc. 119:4841-4845, 1997; Ghosh et al, Chem. Biol. 5:439-445, 1997; Hamuro et al., Angew. Chem. Int. Ed. Engl. 36:2680-2683, 1997; Alberg et al., Science 262:248-250, 1993; Tauton et al., J. Am. Chem. Soc. 118:10412-10422, 1996; Park et al., J. Am. Chem. Soc. 121:8-13, 1999; Prasanna et al., Biochemistry 37:6883-6893, 1998; Tiley et al., J. Am. Chem. Soc. 119:7589-7590, 1997; Judice et al., PNAS, USA 94:13426-13430, 1997; Fan et al., J. Am. Chem. Soc. 120:8893-8894, 1998; Gamboni et al., Biochemistry 37:12189-12194, 1998, each incorporated herein by reference). Briefly, these methods involve rational design and selection of peptides and mimetics that effectively block interactions between selected biologically active peptides or proteins, whereby the selected peptides and mimetics significantly reduce aggregation of the active peptides or proteins in a mucosal formulation or when the peptide or protein is otherwise coordinately administered with a dopamine receptor agonist. Anti-aggregation peptides and mimetics thus identified are in turn coordinately administered with, or admixed or conjugated in a combinatorial formulation with, the biologically active peptide or protein to effectively inhibit aggregation of the active peptide or protein in a manner that significantly enhances absorption and/or bioavailability of the dopamine receptor agonist.

Other anti-aggregation agents for use within the invention include chaperonins and analogs and mimetics of such molecules, as well as antibodies and antibody fragments that function in a similar, but often more specific manner than chaperonins to bind peptide and protein domains and thereby block associative interactions there between. These molecular chaperones were initially recognized as stress proteins produced in cells requiring repair. In particular, studies of heat shock on enzymes showed that molecular chaperones function not only during cellular stress but also to chaperone the process of normal protein folding. Chaperonins comprise an ubiquitous family of proteins that mediate post-translational folding and assembly of other proteins into oligomeric structures. They prevent the formation of incorrect structures, and also act to disrupt incorrect structures that form during these processes. The chaperones non-covalently bind to the interactive surface of a target protein. This binding is reversed under circumstances that favor the formation of the correct structure by folding. Chaperones have not been shown to be specific for only one protein, but rather act on families of proteins which have similar stoichiometric requirements (e.g., specific structural domains that are recognized by the chaperones). Various publications describe the selection and use of chaperoning, antibodies and antibody fragments as aggregation-blocking agents for use within the invention (see, e.g., WO 93/11248; WO 93/13200; WO 94/08012; WO; WO 94/11513; WO 94/08012; and U.S. Pat. No. 5,688,651, each incorporated herein by reference).

Additional methods for inhibiting aggregation within the methods and compositions of the invention include the use of fusion proteins, as disclosed for example for IGF-I (EP 130,166; U.S. Pat. No. 5,019,500; and EP 219,814, each incorporated herein by reference). These incorporated references disclose expression of fusion peptides of IGF-I with a protective polypeptide in bacteria. EP 264,074 discloses a two-cistronic met-IGF-I expression vector with a protective peptide of 500-50,000 molecular weight (see also, U.S. Pat. No. 5,028,531; and Saito et al., J. Biochem., 101:1281-1288, 1987, each incorporated herein by reference). Other fusion techniques include fusion of IGF-1 with a protective peptide from which a rop gene is cut off (EP 219,814, incorporated herein by reference), in which IGF-I is multimerized (Schulz et al., J. Bacteriol., 169:5385-5392, 1987, incorporated herein by reference), in which IGF-I is fused with luteinizing hormone (LH) through a chemically cleavable methionyl or tryptophan residue at the linking site (Saito et al., J. Biochem., 101:123-134, 1987, incorporated herein by reference), and in which IGF-I is fused with superoxide dismutase (EP 196,056; Niwa et al., Ann. NY Acad. Sci., 469:31-52, 1986, incorporated herein by reference). These disclosures, which teach chemical synthesis, cloning, and successful expression of genes for IGF-I fused to another polypeptide, are generally applicable to prepare a range of fusion polypeptides with other therapeutic peptides and proteins for use within the invention. Alternatively, dopamine receptor agonists can be chemically coupled in a similar manner with a carrier or targeting peptide or protein that may in turn be protected by fusion to yet another peptide or protein according to these and related methods.

Yet additional methods for use within the mucosal deliver formulations and coordinate administration methods involve addition of a leader sequence to the subject therapeutic peptide or protein to improve the fidelity of folding after recombinant expression. In this context, U.S. Pat. No. 5,158,875 (incorporated herein by reference) describes a method for refolding recombinant IGF-I that involves cloning the IGF-I gene with a positively charged leader sequence prior to transfecting the DNA into the host cell. The additional positive charge on the amino terminus of the recombinant IGF-I promotes correct refolding when the solubilized protein is stirred for 2-16 hours in denaturant solution. Following refolding, the leader sequence is cleaved and the active recombinant protein is purified.

Yet another method for facilitating in vitro refolding of recombinant polypeptides involves using a solubilized affinity fusion partner, for example comprising two IgG-binding domains derived from staphylococcal protein A (see, e.g., Samuelsson et al., Bio/Technology, 9:731, 1991, incorporated herein by reference). This method uses the protein A domain as a solubilizer of misfolded and multimeric IGF-I. While this method does not use denaturing agents or redox chemicals, it involves the added steps of fusing onto the IGF-I gene a separate gene and removing the polypeptide encoded by that gene after expression of the fusion gene.

Other techniques in peptide and protein engineering disclosed herein will further reduce the extent of protein aggregation and instability in mucosal formulations of the invention. While such resultant engineered or modified proteins may be regarded as new entities in regards to regulatory implications, they retain their suitability for use within the present invention. One example of a useful peptide or protein modification in this context is PEGylation. The stability and aggregation problems of polypeptide drugs can be significantly improved by covalently conjugating water-soluble polymers such as PEG with the polypeptide. Another example is the modification of the peptide or protein amino acid sequence in terms of the identity or location of one or more residues, e.g., by terminal or internal addition, deletion or substitution (e.g., deletion of cysteine residues or replacement by alanine or serine) to reduce aggregation potential. The improvements in terms of stability and aggregation potential that are achieved by these methods enables a therapeutically effective polypeptide or protein to be continuously released over a prolonged period of time following a single administration of the pharmaceutical composition to a subject.

Charge Modifying and pH Control Agents and Methods

To improve the transport characteristics of dopamine receptor agonists and other active agents, for example macromolecular drugs, peptides and proteins, across hydrophobic mucosal membrane barriers, the invention also provides techniques and reagents for charge modification of selected agents within mucosal delivery formulations and method. In this regard, the relative permeabilities of macromolecules can be related to their partition coefficients. The degree of ionization of molecules, which is dependent on the pKₐ of the molecule and the pH at the mucosal membrane surface also affects permeability of the molecules.

Permeation and partitioning of biologically active agents, including dopamine receptor agonists, for mucosal delivery within the methods and compositions of the invention is facilitated by charge alteration or charge spreading of the active agent, which is achieved according to known methods, for example, by alteration of charged functional groups, by modifying the pH of the delivery vehicle or solution in which the active agent is delivered, or by coordinate administration of a charge- or pH-altering reagent with the active agent.

A model compound for evaluating charge- and pH-modification methods for use within the mucosal delivery formulations and methods of the inventions is nicotine. The charge status of this model therapeutic as a function of pH has been investigated at various delivery sites of skin and absorptive mucosae (see, e.g., Nair et al., J. Pharm. Sci. 86:257-262, 1997, incorporated herein by reference). Nicotine is a diacidic base with well-separated pKₐ values (3.04 and 7.84) that allow the study of particular species by pH control. The dissociation of nicotine follows the pH-partition hypothesis, so the theoretical relative proportions of the different charged species at any particular pH can be determined. As an ionizable compound (pKₐ values of 3.04 and 7.84), nicotine in solutions of different pH values provides a model for determining the influence of the charge status of a molecule on permeation.

The permeation of nicotine across certain mucosal and skin surfaces follows zero-order kinetics. The rate of permeation is dependent on donor solution pH and increases exponentially as the pH of the delivery solution is increased. As expected with a majority of charged macromolecular species for use within the invention, the permeability of nicotine across various skin and mucosal surfaces is reportedly higher un-ionized species (NN) than for ionized species (NNH⁺, NH⁺NH⁺). It is also reported that un-ionized nicotine molecules are more permeable through absorptive mucosae (nasal, buccal, sublingual, and gingival) than through skin (abdominal, dorsal, thigh, and ear pinna). Partition studies confirm that biomembrane permeation of nicotine follows the pH-partition theory.

Consistent with these general teachings, intranasal delivery of charged macromolecular species, including dopamine receptor agonists and peptide and protein therapeutics, within the methods and compositions of the invention is substantially improved when the active agent is delivered to the mucosal surface in a substantially un-ionized, or neutral, electrical charge state.

Calculation of the isoelectric points of dopamine receptor agonists as well as native peptides and proteins is readily undertaken to guide the selection of pH and other values for mucosal formulations within the invention, which optionally deliver charged macromolecules in a substantially un-ionized state to the mucosal surface or, alternatively, following mucosal delivery at a target site of drug action. The pI of an amphoteric molecule is defined as the pH at which the net charge is zero. The variation of net charge with pH is of importance in charge-dependent separation methods like electrophoresis, isoelectric focusing, chromatofocusing and ion-exchange chromatography. Thus, methods for estimating isoelectric points (pl) for native peptides and proteins are well known and readily implemented within the methods and compositions of the invention (see, e.g., Cameselle, et al., Biochem. Educ. 14:131-136, 1986; Skoog, et al., Trends Anal. Chem. 5:82-83, 1986; Sillero et al., Anal. Biochem. 179:319-25, 1989; Englund, et al., Biochim. Biophys. Acta, 1065:185-194, 1991; Bjellquist et al., Electrophoresis. 14:1023-1031, 1993; Mosher et al., J. Chromatogr. 638:155-164, 1993; Bjellqvist et al., Electrophoresis 15:529-539, 1994; Watts, et al., Electrophoresis 16:22-27, 1995, each incorporated herein by reference).

For determining pI values of peptides and proteins for use within the invention, net charge can be estimated, for example, by the well-known Henderson-Hasselbalch equation. These determinations are based in part on the amino acid composition of the subject peptide or protein, yielding component pI values for specific amino acid side chains and for the N- and C-terminal groups. The individual ionizable side chains of each type of amino acid are typically assumed to have pKa values distributed around the projected pKa, value, simulating the situation in polypeptides and proteins where a given type of ionizable amino acid side chain often appears in several positions in the amino acid sequence and with various individual ionization constants, depending both on the adjacent side chains and on the three-dimensional environment in the protein (see, e.g., Bjellqvist et al., Electrophoresis 15:529-539, 1994; Matthew, Annu. Rev. Biophys. Chem. 14:387-417, 1985, each incorporated herein by reference). By assuming a distribution of pKa values, the calculated titration curves will be smoothed out. The presence of other charged groups is also taken into account. These analyses yield a set of pKa values, including values for amino acid residues with ionizable side chains. Each particular type of ionizable group is assumed to have pKa values distributed around the chosen value, thereby simulating the situation in intact proteins and polypeptides. According to these known calculation methods, accurate estimates of pI values for peptides and proteins show sufficient agreement with experimental values determined for native proteins, over a wide pH range (3.4-11), particularly when more refined analyses, including such factors as charge contributions of heme groups, sialic acid residues, etc., are taken into account (see, e.g., Henriksson et al., Electrophoresis. 16:1377-1380, 1995, incorporated herein by reference).

Thus, for polypeptides of known amino acid composition, a sufficient pI value estimate can be calculated by use of the ionization constant pKa for amino acid side chain groups. Where other types of ionizable groups occur, the charge for each such group at any given pH can also be readily estimated. The total net charge at a selected pH is obtained by summing up the charge for each type of ionizable group times the number of groups. In the present study, suitable average pKa, values were selected for the ionizable amino acid side chains, and for the terminal groups. Additional guidance for determining pI values for polypeptides and other therapeutic molecules useful within the invention is provided, for example, by Englund, et al., Biochim. Biophys. Acta, 1065:185-194, 1991; Englund et al., Electrophoresis 14:1307-1311, 1993; Uzcategui et al., J. Biotechnol. 19:271-286, 1991; Sims et al., Gene 74:411-422, 1988; Cameselle, et al., Biochem. Educ. 14:131-136, 1986; Skoog, et al., Trends Anal. Chem. 5:82-83, 1986; Sillero et al., Anal. Biochem. 179:319-25, 1989; Bjellquist et al., Electrophoresis. 14:1023-1031, 1993; Mosher et al., J. Chromatogr. 638:155-164, 1993; Bjellqvist et al., Electrophoresis 15:529-539, 1994; Watts, et al., Electrophoresis 16:22-27, 1995; and Oda et al., Biochemistry 33:5275-5284, 1994 (each incorporated herein by reference). These and other teachings in the art allow for sufficiently accurate determination of charge values and ready determination of appropriate pH values and other modifications to components of mucosal delivery formulations within the invention to facilitate mucosal delivery of dopamine receptor agonista and other therapeutic agents in a substantially unionized form. Naturally, pH adjustments and other modifications to alter the charge status of a given therapeutic compound are determined in such a manner as to preserve substantial biological activity of the therapeutic compound within the formulation or after delivery at a target site of action.

Certain dopamine receptor agonists and other therapeutic agents and non-therapeutic components of mucosal formulations for use within the invention will be charge modified to achieve a cationized state in a mucosal formulation or at the target site for drug action. Cationization offers a convenient means of altering the biodistribution and transport properties of proteins and macromolecules within the invention. In many cases, cationized molecules have higher organ uptake and penetration compared with non-cationized forms (see, e.g., Ekrami et al., Journal of Pharmaceutical Sciences 84:456-461, 1995; Bergman et al., Clin. Sci. 67:35-43, 1984; Triguero et al., J. Pharm. Exp. Ther. 258:186-192, 1991). In some cases, cationized proteins can penetrate physiological barriers considered impenetrable by the native proteins. For example, cationized albumin (Pardridge et al., J. Pharm. Exp. Ther. 255:893-899, 1991, incorporated herein by reference) and cationized IgG (Triguero et al., Proc. Nat. Acad. Sci. USA, 86:4761-4765, 1989, incorporated herein by reference) have been demonstrated to bind to the brain capillary endothelium in vitro and cross the blood-brain barrier in vivo to a much greater extent than native albumin and native IgG. Cationized proteins are also generally taken up by the lungs to a greater extent than native proteins (Bergman et al., Clin. Sci. 67:35-43, 1984; Triguero et al., J. Pharm. Exp. Ther. 258:186-192, 1991; Pardridge et al., J. Pharm. Exp. Ther. 251:821-826, 1989, each incorporated herein by reference). At the tissue level, it has been demonstrated that cationized ferritin (CF) binds to and is transcytosed across the pulmonary endothelium (Pietra et al., Lab Invest. 49:54-61, 1983; Pietra et al., Lab Invest. 59:683-691, 1988) in isolated, perfused rat lungs, whereas native ferritin does not bind to the pulmonary endothelium and is only transcytosed across this barrier to a small degree. Bergman et al. (Clin. Sci. 67:35-43, 1984, incorporated herein by reference) demonstrated that by increasing the level of cationization and the charge density of human serum albumin (as measured by the change in the pI value of native albumin), the uptake of cationized albumins by the lungs following iv administration in rats can be increased. Pardridge et al. have also demonstrated that cationized IgG and physiologically cationic histone (Pardridge et al., J. Pharm. Exp. Ther. 251:821-826, 1989, incorporated herein by reference) have higher uptakes in the lungs compared with native IgG and bovine albumin, respectively. However, some studies have failed to demonstrate higher lung uptake for cationized proteins compared with native proteins. For instance, Pardridge et al (Pardridge et al., J. Pharm. Exp. Ther. 255:893-899, 1991, incorporated herein by reference) and Takakura et al.(Takakura et al., Pharm. Res. 7:339-346, 1990, incorporated herein by reference) report lower lung uptake for cationized albumin compared with native albumin following iv biodistribution studies in animals.

In accordance with these teachings, selected dopamine receptor agonists and/or other active or inactive components of mucosal formulations within the invention will be subject to charge modifications that yield an increase in the positive charge density of the charge modified molecule. These modifications extend also to cationization of peptide and protein conjugates, carriers and other delivery forms for enhancing mucosal delivery of dopamine receptor agonist disclosed herein. Cationization of biologically active agents and other formulation components in this context is undertaken in a manner that substantially preserves the biological activity of the active agent and limits potentially adverse side effects, including tissue damage and toxicity.

Degradative Enzyme Inhibitory Agents and Methods

A major drawback to effective mucosal delivery of biologically active agents, including dopamine receptor agonists, is that they are subject to degradation by mucosal enzymes. The oral route of administration of therapeutic compounds is particularly problematic, because in addition to proteolysis in the stomach, the high acidity of the stomach destroys many active and inactive components of mucosal delivery formulations before they reach an intended target site of drug action. Further impairment of activity occurs by the action of gastric and pancreatic enzymes, and exo and endopeptidases in the intestinal brush border membrane, and by metabolism in the intestinal mucosa where a penetration barrier substantially blocks passage of the active agent across the mucosa.

In addition to their susceptibility to enzymatic degradation, many therapeutic compounds, particularly relatively low molecular weight proteins, and peptides, introduced into the circulation, are cleared quickly from mammalian subjects by the kidneys. This problem may be partially overcome by administering large amounts of the therapeutic compound through repeated administration. However, higher doses of therapeutic formulations containing protein or peptide components can elicit antibodies that can bind and inactivate the protein and/or facilitate the clearance of the protein from the subject's body. Repeated administration of the formulation containing the therapeutic protein or peptide is essentially ineffective and can be dangerous as it can elicit an allergic or autoimmune response.

The problem of metabolic lability of therapeutic compounds may be addressed in part through rational drug design. However, medicinal chemists have had less success in manipulating the structures of peptides and proteins to achieve high cell membrane permeability while still retaining pharmacological activity. Unfortunately, many of the structural features of peptides and proteins (e.g., free N-terminal amino and C-terminal carboxyl groups, and side chain carboxyl (e.g., Asp, Glu), amino (e.g., Lys, Arg) and hydroxyl (e.g. Ser, Thr, Tyr) groups) that bestow upon the molecule affinity and specificity for its pharmacological binding partner also bestow upon the molecule undesirable physicochemical properties (e.g., charge, hydrogen bonding potential) which limit their cell membrane permeability. Therefore, alternative strategies need to be considered for intranasal formulation and delivery of peptide and protein therapeutics.

Attempts to overcome the so-called enzymatic barrier to drug delivery include the use of liposomes (Takeuchi et al., Pharm. Res. 13:896-901, 1996, incorporated herein by reference) and nanoparticles (Mathiowitz et al., Nature. 386:410-4, 1997, incorporated herein by reference) that reportedly provide protection for incorporated insulin towards an enzymatic attack and the development of delivery systems targeting to the colon, where the enzymatic activity is comparatively low (Rubenstein et al., J. Control Rel. 46:59-73, 1997, incorporated herein by reference). In addition, co-administration of protease inhibitors has been reported in various studies to improve the oral bioavailability of insulin (Fujii et al, J. Pharm Pharmacol. 37:545-9, 1985; Yamamoto et al., Pharm Res. 11:1496-600, 1994; Moroshita et al., Int. J. Pharm. 78:9-16, 1992, incorporated herein by reference).

Thus, in recent years the use of enzyme inhibitors to overcome the enzymatic barrier to perorally administered therapeutic compounds has gained considerable interest (for a detailed review, see, Bernkop-Schnürch, A. J. Control. Rel. 52:1-16, 1998, incorporated herein by reference. However, especially for peptide and protein drugs which are used in long-term therapy, the co-administration of enzyme inhibitors remains questionable because of side effects caused by these agents. Several side effects, such as systemic intoxications, a disturbed digestion of nutritive proteins, and hypertrophy as well as hyperplasia of the pancreas based on a feedback regulation, may accompany enzyme inhibitor co-administration by oral delivery methods. Even if systemic toxic side effects and an intestinal mucosal damage can be excluded, enzyme inhibitors of pancreatic proteases still have a toxic potential caused by the inhibition of these digestive enzymes themselves. Besides a disturbed digestion of nutritive proteins, an inhibitor-induced stimulation of protease secretion caused by a feed-back regulation has to be expected (Reseland et al., Hum. Clin. Nutr. 126:634-642, 1996, incorporated herein by reference). Numerous studies have investigated this feed-back regulation with inhibitors, such as Bowman-Birk inhibitor, soybean trypsin inhibitor (Kunitz trypsin inhibitor) and camostat, in rats and mice. They demonstrate that this feed-back regulation rapidly leads to both hypertrophy and hyperplasia of the pancreas. Moreover, a prolonged oral administration of the Bowman-Birk inhibitor and soybean trypsin inhibitor leads to the development of numerous neoplastic foci, frequently progressing to invasive carcinoma (Otsuki et al., Pancreas 2:164-169, 1987; Melmed et al., Biochim. Biophys. Acta 421:280-288, 1976; McGuinness et al. Scand. J. Gastroneterol. 17:273-277, 1982; Ge et al., Br. J. Nutr. 70:333-345, 1993, each incorporated herein by reference). A reduction or even exclusion of this feed-back regulation might be possible by the development of drug delivery systems which keep inhibitor(s) concentrated on a restricted area of the intestine, where drug liberation and subsequent absorption takes place. For a general review of more recent enzyme inhibitor strategies in the context of oral peptide drug delivery, see, e.g., Marschütz et al., Biomaterials 21:1499-1507, 2000 (incorporated herein by reference).

The present invention provides processing methods and combinatorial formulations directed toward coordinate administration of a dopamine receptor agonist, optionally formulated with a peptide or protein component that enhances mucosal delivery of the dopamine receptor agonist, with an enzyme inhibitor. Since a variety of degradative enzymes are present in mucosal environments, the prophylactic and therapeutic compositions and methods of the invention are readily modified to incorporate the addition or coadministration of an enzyme inhibitor, such as a protease inhibitor, with the dopamine receptor agonist (e.g., which is optionally formulated also with a physiologically active peptide or protein), to thereby improve bioavailability of the dopamine receptor agonist (either by protecting the dopamine receptor agonist or another active or delivery-enhancing agent from degradative effects). For example, in certain cases where therapeutically active peptides and proteins are formulated or coordinately administered with the dopamine receptor agonist, one or more protease inhibiting agents is/are optionally combined or coordinately administered in the formulation or method for mucosal delivery. In certain embodiments, the enzyme inhibitor is admixed with or bound to a common carrier with the dopamine receptor agonist and/or other active or inactive formulation component, such as a protein or peptide formulation component. For example, an inhibitor of proteolytic enzymes may be incorporated in a therapeutic or prophylactic formulation of the invention to protect a mucosal delivery-enhancing protein or peptide from proteolysis, and thereby enhance bioavailability of the dopamine receptor agonist.

Any inhibitor which inhibits the activity of an enzyme to protect the dopamine receptor agonist or other biologically active or inactive formulation component (s) may be usefully employed in the compositions and delivery methods of the invention. Useful enzyme inhibitors for the protection of biologically active proteins and peptides include, for example, soybean trypsin inhibitor, pancreatic trypsin inhibitor, chymotrypsin inhibitor and trypsin and chrymotrypsin inhibitor isolated from potato (solanum tuberosum L.) tubers. A combination or mixtures of inhibitors may be employed. Additional inhibitors of proteolytic enzymes for use within the invention include ovomucoid-enzyme, gabaxate mesylate, alpha1-antitrypsin, aprotinin, amastatin, bestatin, puromycin, bacitracin, leupepsin, alpha2-macroglobulin, pepstatin and egg white or soybean trypsin inhibitor. These and other inhibitors can be used alone or in combination. The inhibitor(s) may be incorporated in or bound to a carrier, e.g., a hydrophilic polymer, coated on the surface of the dosage form which is to contact the nasal mucosa, or incorporated in the superficial phase of said surface, in combination with the biologically active agent or in a separately administered (e.g., pre-administered) formulation.

The amount of the inhibitor, e.g., of a proteolytic enzyme inhibitor, that is optionally incorporated in the compositions of the invention will vary depending on (a) the properties of the specific inhibitor, (b) the number of functional groups present in the molecule which may be reacted to introduce ethylenic unsaturation necessary for copolymerization with the hydrogel forming monomers, and (c) the number of lectin groups, such as glycosides, which are present in the inhibitor molecule. It may also depend on the specific therapeutic agent which is intended to be administered. Generally speaking, a useful amount of an enzyme inhibitor is from about 0.1 mg/ml to about 50 mg/ml, often from about 0.2 mg/ml to about 25 mg/ml, and more commonly from about 0.5 mg/ml to 5 mg/ml of the of the formulation (i.e., a separate protease inhibitor formulation or combined formulation with the inhibitor and biologically active agent).

With the necessary caveat of determining and considering possible toxic and other deleterious side effects, various inhibitors of mucosally-present enzymes may be evaluated for use within the mucosal delivery methods and compositions of the invention. In the case of trypsin inhibition, suitable inhibitors may be selected from, e.g., aprotinin, BBI, soybean trypsin inhibitor, chicken ovomucoid, chicken ovoinhibitor, human pancreatic trypsin inhibitor, camostat mesilate, flavonoid inhibitors, antipain, leupeptin, p-aminobenzamidine, AEBSF, TLCK (tosyllysine chloromethylketone), APMSF, DFP, PMSF, and poly(acrylate) derivatives. In the case of chymotrypsin inhibition, suitable inhibitors may be selected from, e.g., aprotinin, BBI, soybean trypsin inhibitor, chymostatin, benzyloxycarbonyl-Pro-Phe-CHO, FK-448, chicken ovoinhibitor, sugar biphenylboronic acids complexes, DFP, PMSF, β-phenylpropionate, and poly(acrylate) derivatives. In the case of elastase inhibition, suitable inhibitors may be selected from, e.g., elastatinal, methoxysuccinyl-Ala-Ala-Pro-Val-chloromethylketone (MPCMK), BBI, soybean trypsin inhibitor, chicken ovoinhibitor, DFP, and PMSF. Other naturally occurring, endogenous enzyme inhibitors for additional known degradative enzymes present in mucosal environments, or alternatively present in preparative materials for production of mucosal delivery formulations, will be readily ascertained by those skilled in the art for incorporation within the methods and compositions of the invention.

Additional enzyme inhibitors for use within the invention are selected from a wide range of non-protein inhibitors which vary in their degree of potency and toxicity (see, e.g., L. Stryer, Biochemistry, W H Freeman and Company, NY, N.Y., 1988). As described in further detail below, immobilization of these adjunct agents to matrices or other delivery vehicles, or development of chemically modified analogues, may be readily implemented to reduce or even eliminate toxic effects, when they are encountered. Among this broad group of candidate enzyme inhibitors for use within the invention are organophosphorous inhibitors, such as diisopropylfluorophosphate (DFP) and phenyhnethylsulfonyl fluoride (PMSF), which are potent, irreversible inhibitors of serine proteases (e.g., trypsin and chymotrypsin). The additional inhibition of acetylcholinesterase by these compounds makes them highly toxic in uncontrolled delivery settings (L. Stryer, Biochemistry, W H Freeman and Company, NY, N.Y., 1988). Another candidate inhibitor, 4-(2-Aminoethyl)-benzenesulfonyl fluoride (AEBSF), has an inhibitory activity comparable to DFP and PMSF, but it is markedly less toxic. (4-Aminophenyl)-methanesulfonyl fluoride hydrochloride (APMSF) is another potent inhibitor of trypsin, but is toxic in uncontrolled settings. In contrast to these inhibitors, 4-(4-isopropylpiperadinocarbonyl)phenyl 1,2,3,4,-tetrahydro-1-naphthoate methanesulphonate (FK-448) is a low toxic substance, representing a potent and specific inhibitor of chymotrypsin. The co-administration of this compound led to an enhanced intestinal absorption of insulin in rats and dogs, resulting in a decrease in blood glucose level. This increased bioavailability of insulin was found to be related to the inhibition of digestive enzymes, especially chymotrypsin (Fujii et al., J. Pharm. Pharmacol. 37:545-549, 1985). Further representatives of this non-protein group of inhibitor candidates, and also exhibiting low toxic risk, are camostat mesilate (N,N′-dimethyl carbamoylmethyl-p-(p′-guanidino-benzoyloxy)phenylacetate methane-sulphonate) (Yamamoto et al., Pharm. Res. 11:1496-1500, 1994, incorporated herein by reference) and Na-glycocholate (Yamamoto et al., Pharm. Res. 11: 1496-1500, 1994; Okagava et al., Life Sci. 55:677-683, 1994, incorporated herein by reference).

Yet another type of enzyme inhibitory agent for use within the methods and compositions of the invention are amino acids and modified amino acids that interfere with enzymatic degradation of specific therapeutic compounds. For use in this context, amino acids and modified amino acids are substantially non-toxic and can be produced at a low cost. However, due to their low molecular size and good solubility, they are readily diluted and absorbed in mucosal environments. Nevertheless, under proper conditions, amino acids can act as reversible, competitive inhibitors of protease enzymes (see, e.g., McClellan et al., Biochim. Biophys Acta 613:160-167, 1980, incorporated herein by reference). Certain modified amino acids can display a much stronger inhibitory activity. A desired modified amino acid in this context is known as a ‘transition-state’ inhibitor. The strong inhibitory activity of these compounds is based on their structural similarity to a substrate in its transition-state geometry, while they are generally selected to have a much higher affinity for the active site of an enzyme than the substrate itself. Transition-state inhibitors are reversible, competitive inhibitors. Examples of this type of inhibitor are α-aminoboronic acid derivatives, such as boro-leucine, boro-valine and boro-alanine. The boron atom in these derivatives can form a tetrahedral boronate ion which is believed to resemble the transition state of peptides during their hydrolysis by aminopeptidases. These amino acid derivatives are potent and reversible inhibitors of aminopeptidases and it is reported that boro-leucine is more than 100-times more effective in enzyme inhibition than bestatin and more than 1000-times more effective than puromycin (Hussain et al., Pharm. Res. 6:186-189, 1989). Another modified amino acid for which a strong protease inhibitory activity has been reported is N-acetylcysteine, which inhibits enzymatic activity of aminopeptidase N (Bernkop-Schnurch et al., Pharm. Res. 14:181-185, 1997, incorporated herein by reference). This adjunct agent also displays mucolytic properties that can be employed within the methods and compositions of the invention to reduce the effects of the mucus diffusion barrier (Bernkop-Schnurch et al., Pharm. Sci 2:361-363, 1996, incorporated herein by reference).

Still other useful enzyme inhibitors for use within the coordinate administration, multi-processing and/or combinatorial formulation methods and compositions of the invention may be selected from peptides and modified peptide enzyme inhibitors. An important representative of this class of inhibitors is the cyclic dodecapeptide, bacitracin, obtained from Bacillus licheniformis. Bacitracin A has a molecular mass of 1423 Da and shows remarkable resistance against the action of proteolytic enzymes like trypsin and pepsin (Hickey, R. J., Prog. Ind. Microbiol. 5:93-150, 1964, incorporated herein by reference). It has several biological properties inhibiting bacterial peptidoglycan synthesis, mammalian transglutaminase activity, and proteolytic enzymes such as aminopeptidase N. Because of its protease inhibitory activity, it has been used to inhibit the degradation of various therapeutic (poly)peptides, such as insulin, metkephamid, LH-RH, and buserelin (Yamamoto et al., Pharm. Res. 11:1496-1500, 1994; Langguth et al., J. Pharm. Pharmacol. 46:34-40, 1994; Raehs, et al., Pharm. Res. 5:689-693, 1988, each incorporated herein by reference). Besides its inhibitory activity, bacitracin also displays absorption-enhancing effects without leading to a serious intestinal mucosal damage (Gotoh et al., Biol. Pharm. Bull. 18:794-796, 1995, incorporated herein by reference).

Nevertheless, bacitracin may not be useful in certain uncontrolled delivery settings due to its established nephrotoxicity. To date, it has almost exclusively been used in veterinary medicine and as a topical antibiotic in the treatment of infections in man. Covalent linkage of bacitracin to a mucoadhesive polymer (carbomer) has been shown to conserve the inhibitory activity of the compound within the carrier matrix (Bernkop-Schnurch et al., Pharm. Res. 14:181-185, 1997, incorporated herein by reference).

In addition to these types of peptides, certain dipeptides and tripeptides display weak, non-specific inhibitory activity towards some proteases (Langguth et al., J. Pharm. Pharmacol. 46:34-40, 1994, incorporated herein by reference). By analogy with amino acids, their inhibitory activity can be improved by chemical modifications. For example, phosphinic acid dipeptide analogues are also ‘transition-state’ inhibitors with a strong inhibitory activity towards aminopeptidases. They have reportedly been used to stabilize nasally administered leucine enkephalin (Hussain et al., Pharm. Res. 9:626-628, 1992).

Another example of a transition-state analogue is the modified pentapeptide pepstatin (McConnell et al., J. Med. Chem. 34:2298-2300, 1991, incorporated herein by reference), which is a very potent inhibitor of pepsin. Structural analysis of pepstatin, by testing the inhibitory activity of several synthetic analogues, demonstrated the major structure-function characteristics of the molecule responsible for the inhibitory activity (McConnell et al., J. Med. Chem. 34:2298-2300, 1991, incorporated herein by reference). Similar analytic methods can be readily applied to prepare modified amino acid and peptide analogs for blockade of selected, intranasal degradative enzymes.

Another special type of modified peptides are inhibitors with a terminally located aldehyde function in their structure. For example, the sequence benzyloxycarbonyl-Pro-Phe-CHO, which fulfill the known primary and secondary specificity requirements of chymotrypsin, has been found to be a potent reversible inhibitor of this target proteinase (Walker et al., Biochem. J. 321-323, 1993, incorporated herein by reference). The chemical structures of further inhibitors with a terminally located aldehyde function, e.g. antipain, leupeptin, chymostatin and elastatinal, are also known in the art, as are the structures of other known, reversible, modified peptide inhibitors, such as phosphoramidon, bestatin, puromycin and amastatin

Due to their comparably high molecular mass, polypeptide protease inhibitors are more amenable to smaller compounds to concentrated delivery in a drug-carrier matrix. The advantages of a slow release carrier system for delivery of enzyme inhibitors have been discussed by Kimura et al. (Biol. Pharm. Bull. 19:897-900, 1996, incorporated herein by reference). In this study a mucoadhesive delivery system exhibited a desired release rate of the protease inhibitor aprotinin of approximately 10% per hour, which was almost synchronous with the release rate of a polypeptide drug. In vivo studies with this delivery system showed an improved bioavailability of the drug (Ld.) For this reason, and due to their low toxicity and strong inhibitory activity, polypeptide protease inhibitors will often be selected for use within the mucosal delivery methods and compositions of the invention.

Additional agents for enzyme inhibition within the formulations and methods of the invention involve the use of complexing agents. These agents mediate enzyme inhibition by depriving the intranasal environment (or preparative or therapeutic composition) of divalent cations which are co-factors for many degradative enzymes. For instance, the complexing agents EDTA and DTPA as coordinately administered or combinatorially formulated adjunct agents, in suitable concentration, will be sufficient to inhibit selected degradative enzymes to thereby enhance mucosal delivery of dopamine receptor agonists according to the invention. Further representatives of this class of inhibitory agents are EGTA, 1,10-phenanthroline and hydroxychinoline (Ikesue et al., Int. J. Pharm. 95:171-9, 1993; Garner et al., Biochemistry 13:3227-3233, 1974; Sangadala et al., J. Biol. Chem. 269:10088-10092, 1994; Mizuma et al., Biochim. Biophys. Acta. 1335:111-119, 1997, each incorporated herein by reference). In addition, due to their propensity to chelate divalent cations, these and other complexing agents are useful within the invention as direct, absorption-promoting agents (see, e.g., Lee, V. H. L., J. Control Release 13:213-334, 1990, incorporated herein by reference).

As noted in more detail elsewhere herein, it is also contemplated to use various polymers, particularly mucoadhesive polymers, as enzyme inhibiting agents within the coordinate administration, processing and/or combinatorial formulation methods and compositions of the invention. For example, poly(acrylate) derivatives, such as poly(acrylic acid) and polycarbophil, can affect the activity of various degradative enzymes, for example trypsin and chymotrypsin. The inhibitory effect of these polymers may also be based on the complexation of divalent cations such as Ca²⁺ and Zn²⁺ (Luepen et al., Pharm. Res. 12:1293-1298, 1995, incorporated herein by reference). It is further contemplated that these polymers may serve as conjugate partners or carriers for additional enzyme inhibitory agents, as described above. For example, a chitosan-EDTA conjugate has been developed and is useful within the invention that exhibits a strong inhibitory effect towards the enzymatic activity of zinc-dependent degradative enzymes. The mucoadhesive properties of polymers following covalent attachment of other enzyme inhibitors in this context are not expected to be substantially compromised, nor is the general utility of such polymers as a delivery vehicle for biologically active agents within the invention expected to be diminished. On the contrary, the reduced distance between the delivery vehicle and mucosal surface afforded by the mucoadhesive mechanism will minimize presystemic metabolism of the dopamine receptor agonist and other active and inactive formulation components, while the covalently bound enzyme inhibitors remain concentrated at the site of drug delivery, minimizing undesired dilution effects of inhibitors as well as toxic and other side effects caused thereby. In this manner, the effective amount of a coordinately administered enzyme inhibitor can be educed due to the exclusion of dilution effects.

More recent research efforts in the area of protease inhibition for enhanced delivery of biotherapeutic compounds, including peptide and protein therapeutics, has focused on covalent immobilization of enzyme inhibitors on mucoadhesive polymers used as drug carrier matrices (see, e.g., Bernkop-Schnurch et al., Drug Dev. Ind. Pharm. 23:733-40, 1997; Bernkop-Schnurch et al., J. Control. Rel. 47:113-21, 1997; Bernkop-Schnurch et al., J. Drug Tare. 7:55-63, 1999, each incorporated herein by reference). In conjunction with these teachings, the invention provides in more detailed aspects an enzyme inhibitor formulated with a common carrier or vehicle for mucosal delivery of a dopamine receptor agonist and, optionally, one or more additional biologically active or delivery-enhancing agents. Optionally, the enzyme inhibitor is covalently linked to the carrier or vehicle. In certain embodiments, the carrier or vehicle is a biodegradable polymer, for example, a bioadhesive polymer. Thus, for example, a protease inhibitor, such as Bowman-Birk inhibitor (BBI), displaying an inhibitory effect towards trypsin and \acute{α}-chymotrypsin (Birk Y. Int. J. Pept. Protein Res. 25:113-31, 1985, incorporated herein by reference), or elastatinal, an elastase-specific inhibitor of low molecular size, may be covalently linked to a mucoadhesive polymer as described herein. The resulting polymer-inhibitor conjugate exhibits substantial utility as a mucosal delivery vehicle for dopamine receptor agonists formulated or delivered alone or in combination with other biologically active agents or additional delivery-enhancing agents according to the methods and compositions of the invention.

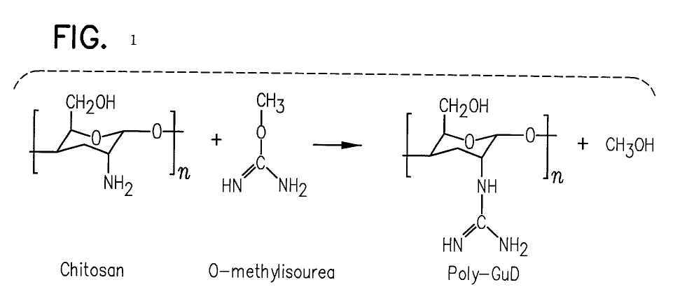

Exemplary mucoadhesive polymer-enzyme inhibitor complexes that are useful within the mucosal delivery formulations and methods of the invention include, but are not limited to: Carboxymethylcellulose-pepstatin (with anti-pepsin activity); Poly(acrylic acid)-Bowman-Birk inhibitor (anti-chymotrypsin); Poly(acrylic acid)-chymostatin (anti-chymotrypsin); Poly(acrylic acid)-elastatinal (anti-elastase); Carboxymethylcellulose-elastatinal (anti-elastase); Polycarbophil—lastatinal (anti-elastase); Chitosan—antipain (anti-trypsin); Poly(acrylic acid)—bacitracin (anti-aminopeptidase N); Chitosan—EDTA (anti-aminopeptidase N, anti-carboxypeptidase A); Chitosan—EDTA—antipain (anti-trypsin, anti-chymotrypsin, anti-elastase) (see, e.g., Bernkop-Schnüarch, J. Control. Rel. 52:1-16, 1998, incorporated herein by reference). As described in further detail below, certain embodiments of the invention will optionally incorporate a novel chitosan derivative or chemically modified form of chitosan. One such novel derivative for use within the invention is denoted as a β-[1→4]-2-guanidino-2-deoxy-D-glucose polymer (poly-GuD) (see, FIG. 1).

Mucolytic and Mucus-Clearing Agents and Methods

Effective delivery of biotherapeutic agents via mucosal administration must take into account the decreased drug transport rate across the protective mucus lining of mucosal tissues, in addition to drug loss due to binding to glycoproteins of the mucus layer. Normal mucus is a viscoelastic, gel-like substance consisting of water, electrolytes, mucins, macromolecules, and sloughed epithelial cells. It serves primarily as a cytoprotective and lubricative covering for the underlying mucosal tissues. In the nasal mucosa and other mucosal tissues, mucus is secreted by randomly distributed secretory cells located in the mucosal epithelium. The structural unit of mucus is mucin. This glycoprotein is mainly responsible for the viscoelastic nature of mucus, although other macromolecules may also contribute to this property. In airway mucus, such macromolecules include locally produced secretory IgA, lgM, IgE, lysozyme, and bronchotransferrin, which also play an important role in host defense mechanisms.

The thickness of mucus varies from organ to organ and between species. However, mucin glycoproteins obtained from different sources have similar overall amino acid and protein/carbohydrate compositions, although the molecular weight may vary over a wide. Mucin consists of a large protein core with oligosaccharide side-chains attached through the O-glycosidic linkage of galactose or N-acetyl glucosamine to hydroxyl groups of serine and threonine residues. Either sialic acid or L-fucose forms the terminal group of the side chain oligosaccharides with sialic acid (negatively charged at pH greater than 2.8) forming 50 to 60% of the terminal groups. The presence of cysteine in the end regions of the mucin core facilitates cross-linking of mucin molecules via disulfide bridge formation.

The presence of a mucus layer that coats all epithelial surfaces has been largely overlooked in the elucidation of epithelial penetration enhancement mechanisms to date. This is partly because the role of mucus in the absorption of peptide and protein drugs has not yet been well established. However, for these and other drugs exhibiting a comparatively high molecular mass, the mucus layer covering mucosal surfaces may represent an almost insurmountable barrier. According to the conventional formula for calculation of the diffusion coefficient, in which the radius of the molecule indirectly correlates with the diffusion coefficient, the mucus barrier increases tremendously for polypeptide drugs. Studies focusing on this so called ‘diffusion barrier’ have demonstrated that proteins of a molecular mass greater than approximately 5 kDa exhibit minimal or no permeation into mucus layers (Allen, et al., ‘Mucus Medicine and Biology’, E. N. Elder, J. B. Elstein (eds.) p. 115, Vol. 144, Plenum Press, New York, 1982; Bernkop-Schnurch., Pharm. Sci. 2:361, 1996, each incorporated herein by reference).

The mucosal delivery formulations and coordinate administration methods of the instant invention optionally incorporate effective mucolytic or mucus-clearing agents, which serve to degrade, thin or clear mucus from mucosal surfaces to facilitate absorption of mucosally administered dopamine receptor agonists and other biotherapeutic and delivery-enhancing agents. Within certain methods of the invention, a mucolytic or mucus-clearing agent is coordinately administered with the dopamine receptor agonist as an adjunct compound to enhance mucosal delivery of the dopamine receptor agonist. Alternatively, an effective amount of a mucolytic or mucus-clearing agent is incorporated as a processing agent within a method for preparing a mucosal delivery formulation of the invention, or as an additive within a combinatorial formulation of the invention, to provide an improved formulation that enhances mucosal delivery of dopamine receptor agonists by reducing the barrier effects of mucosal mucus.

A variety of mucolytic or mucus-clearing agents are available for incorporation within the methods and compositions of the invention (see, e.g., Lee, et al., Crit. Rev. Ther. Drug Carrier Syst. 8:91-192, 1991; Bernkop-Schnurch et al., Arzneimittelforschung, 49:799-803, 1999, each incorporated herein by reference). Based on their mechanisms of action, mucolytic and mucus clearing agents can often be classified into the following groups: proteases (e.g., pronase, papain) that cleave the protein core of mucin glycoproteins; sulfhydryl compounds that split mucoprotein disulfide linkages; and detergents (e.g., Triton X-100, Tween 20) that break non-covalent bonds within the mucus (see, e.g., Allen, A. in ‘Physiology of the Gastrointestinal Tract. L. R. Johnson (ed.), p. 617, Raven Press, New York, 1981, incorporated herein by reference). Additional compounds in this context include, but are not limited to, bile salts and surfactants, for example, sodium deoxycholate, sodium taurodeoxycholate, sodium glycocholate, and lysophosphatidylcholine.

The effectiveness of bile salts in causing structural breakdown of mucus is in the order deoxycholate >taurocholate >glycocholate. Other effective agents that reduce mucus viscosity or adhesion to enhance intranasal delivery according to the methods of the invention include, e.g., short-chain fatty acids, and mucolytic agents that work by chelation, such as N-acylcollagen peptides, bile acids, and saponins (the latter function in part by chelating Ca²⁺ and/or Mg²⁺ which play an important role in maintaining mucus layer structure).

Additional mucolytic agents for use within the methods and compositions of the invention include N-acetyl-L-cysteine (ACS), a potent mucolytic agent that reduces both the viscosity and adherence of bronchopulmonary mucus and is reported to modestly increase nasal bioavailability of human growth hormone in anesthetized rats (from 7.5 to 12.2%) (O'Hagen et al., Pharm. Res., 7:772, 1990, incorporated herein by reference). These and other mucolytic or mucus-clearing agents are contacted with the nasal mucosa, typically in a concentration range of about 0.2 to 20 mM, coordinately with administration of the dopamine receptor agonist, to reduce the polar viscosity and/or elasticity of mucosal mucus.

Still other mucolytic or mucus-clearing agents may be selected from a range of glycosidase enzymes, which are able to cleave glycosidic bonds within the mucus glycoprotein. α-amylase and β-amylase are representative of this class of enzymes, although their mucolytic effect may be limited (Leiberman, J., Am. Rev. Respir. Dis. 97:662, 1967, incorporated herein by reference). In contrast, bacterial glycosidases which allow these microorganisms to permeate mucus layers of their hosts (Corfield et al, Glycoconjugate J. 10:72, 1993, incorporated herein by reference) are highly mucolytic active.

For selecting mucolytic agents for use within the methods and compositions of the invention, it is important to consider the chemical nature of both the mucolytic (or mucus-clearing) and biologically active agents. For example, the proteolytic enzyme pronase exhibits a very strong mucolytic activity at pH 5.0, as well as at pH 7.2. In contrast, the protease papain exhibits substantial mucolytic activity at pH 5.0, but no detectable mucolytic activity at pH 7.2. The reason for these differences in activity are explained in part by the distinct pH-optimum for papain, reported to be pH 5 (Karlson, P., Biochemie, Thieme, Verlag, Stuttgart, New York, 1984, incorporated herein by reference). Thus, mucolytic and other enzymes for use within the invention are typically delivered in formulations having a pH at or near the pH optimum of the subject mucolytic enzyme.

With respect to chemical characterization of the dopamine receptor agonist and other optional biologically active agents, one notable concern is the vulnerability of peptide and protein molecules to the degradative activities of proteases and sulfhydryl. In particular, peptide and protein drugs, conjugate partners, and carriers can be attacked by different types of mucolytic agents. In one study, the mucolytic proteases pronase and papain (which each are endopeptidases that cleave at a high number of bonds) were shown to completely degrade insulin within 2-3 h at pH 7.2 (Bernkop-Schnurch et al., Arzneimittelforschung, 49:799-803, 1999, incorporated herein by reference). In contrast, at pH 2.5 insulin was not at all, or only slightly, degraded by pronase and papain, which can be explained by the pH optimum of both enzymes being far away from pH 2.5. Whereas pronase represents an unusually non-specific protease, papain cleaves after Arg, Lys, Leu, and Gly (Karlson, P., Biochemie, Thieme, Verlag, Stuttgart, New York, 1984, incorporated herein by reference), which are all included in the primary structure of insulin and serve as an additional guide to selection of mucolytic and mucus-clearing agents within the invention.

The presence and number of cysteine residues and disulfide bonds in peptide and protein therapeutics are also important factors to consider in selecting mucolytic or mucus-clearing agents within the invention. When insulin, which displays three disulfide bonds within its molecular structure, is incubated with di-thiothreitol or N-acetylcysteine, there is a rapid degradation of the insulin polypeptide at pH 7.2. A substantially lower degree of degradation at pH 2.5 is attributed to the relatively low amount of reactive thiolate anions (responsible for nucleophilic attack on disulfide bonds) at this pH value (Bernkop-Schnurch et al., Arzneimittelforschung, 49:799-803, 1999).

Whereas it is generally contraindicated to use general proteases such as pronase or papain in combination with peptide or protein drugs and carriers, the practical use of more specific proteases can be undertaken according to the above principals, as can the use of sulfhydryl compounds. For therapeutic polypeptides that exhibit no cysteine moieties within their primary structure (e.g. cyclosporin), the use of sulfhydryl compounds is not problematic. Moreover, even for protein drugs bearing disulfide bonds the use of sulfhydryl compounds can be achieved, particularly where the disulfide bonds are not accessible for thiol attack due to the conformation of the protein, they should remain stable in the presence of this type of mucolytic agents.

For combinatorial use with most dopamine receptor agonists and other biologically active agents within the invention, including peptide and protein therapeutics, non-ionogenic detergents are generally also useful as mucolytic or mucus-clearing agents. These agents typically will not modify or substantially impair the activity of the therapeutic components of the formulation.

Ciliostatic Agents and Methods

Because the self-cleaning capacity of certain mucosal tissues (e.g., nasal mucosal tissues) by mucociliary clearance is necessary as a protective function (e.g., to remove dust, allergens, and bacteria), it has been generally considered that this function should not be substantially impaired by mucosal medications. Mucociliary transport in the respiratory tract is a particularly important defense mechanism against infections (Wasserman., J. Allergy Clin. Immunol. 73:17-19, 1984). To achieve this function, ciliary beating in the nasal and airway passages moves a layer of mucus along the mucosa to removing inhaled particles and microorganisms. During chronic bronchitis and chronic sinusitis, tracheal and nasal mucociliary clearance are often impaired (Warmer., Am. Rev. Respir. Dis. 116:73-125, 1977, incorporated herein by reference). This is presumably due to either excess secretion (Dulfano, et al., Am. Rev. Respir. Dis. 104:88-98, 1971), increased viscosity of mucus (Chen, et al., J. Lab. Clin. Med. 91:423-431, 1978, incorporated herein by reference), alterations in ciliary activity caused by decreased beat frequency (Puchelle et al., Biorheology. 21:265-272, 1984, incorporated herein by reference), loss of portions of the ciliated epithelium (Chodosh et al., Am. Rev. Respir. Dis. 104:888-898, 1971, incorporated herein by reference), or to a combination of these factors. Decreased clearance presumably favors bacterial colonization of respiratory mucosal surfaces, predisposing the subject to infection. The ability to interfere with this host defense system may contribute significantly to a pathological organism's virulence.

As noted above, ciliary activity is a major factor for mucociliary clearance (Duchateau et al., Larynzoscope 95:854-859, 1985, incorporated herein by reference). From patients with “immotile cilia syndrome” it is known that chronic nasal ciliary arrest leads to recurrent infections of the airways (Afzelius., Int. Rev. Exp. Pathol. 19:1-43, 1979, incorporated herein by reference). Many drugs and additives have been shown to adversely impair nasal ciliary movement. For instance, lipophilic and mercuric preservatives, and antihistamines have been demonstrated to induce loss of ciliary function (Hermens, et al., Pharm. Res. 4:445-449, 1987, incorporated herein by reference). In light of these and related findings, it is widely considered that intranasally administered drugs and additives as nasal absorption enhancers should be devoid of any substantial ciliotoxicity.

Ciliated epithelium covers all surfaces in the upper respiratory tract except the entrance to the nose, parts of nasopharynx, pharynx, and larynx that are covered by squamous epithelium, and the olfactory area which has a specialized sensory epithelium. In the human respiratory tract, ciliated cells constitute 30 to 65% of the eight types of epithelial cells. The ratio of ciliated columnar epithelial cells to goblet cells on the airway surface is approximately 5:1.

The Theological characteristics of mucus play a major role in mucociliary clearance. The viscous nature of mucus enables it to trap and retain foreign particles. Beyond a certain limit, however, increase in viscosity may be detrimental to ciliary motility. An intermediate viscosity has been reported to be optimal for mucociliary transport. The ability of a number of chemically dissimilar but rheologically similar substances like guaran, agarose, gelatin, and acrylamide gels to be transported on a mucus-free excised frog palate demonstrates the importance of rheology in mucociliary transport. The demonstration of interdependence between mucociliary clearance and the viscoelastic properties of mucus has been shown by various studies, one of which reported that a representative test material, xanthan gum, exhibits maximum mucociliary clearance in the viscosity range of about 12 to 15 Pa, comparable to respiratory mucus. In addition to viscosity, the efficiency of mucociliary transport is determined in part by the elasticity of the mucus layer. An optimal elastic modulus of 1 Pa has been reported for efficient mucociliary clearance.

Various reports show that mucociliary clearance can be impaired by mucosally administered drugs, as well as by a wide range of formulation additives including penetration enhancers and preservatives. For example, ethanol at concentrations greater than 2% has been shown to reduce the in vitro ciliary beating frequency. This may be mediated in part by an increase in membrane permeability that indirectly enhances flux of calcium ion which, at high concentration, is ciliostatic, or by a direct effect on the ciliary axoneme or actuation of regulatory proteins involved in a ciliary arrest response. Exemplary preservatives (methyl-p-hydroxybenzoate (0.02% and 0.15%), propyl-p-hydroxybenzoate (0.02%), and chlorobutanol (0.5%)) reversibly inhibit ciliary activity in a frog palate model. Other common additives (EDTA (0.1%), benzalkoniuin chloride (0.01%), chlorhexidine (0.01%), phenylinercuric nitrate (0.002%), and phenylmercuric borate (0.002%), reportedly inhibit mucociliary transport irreversibly. Recently, others have investigated the effects of several penetration enhancers including STDHF (0.1 to 1.0%), deoxycholic acid (0.3%), taurocholic acid (0.3%), glycocholic acid (0.3%), and Laureth-9 (0.3%) on the ciliary movement of human adenoid tissue in vitro. Increasing STDHF concentrations from 0.1 to 0.3% reportedly yielded progressive inhibition of ciliary movement, which inhibition was total at 60 minutes and a concentration of 0.3%. Laureth-9 and deoxycholate reportedly inhibited ciliary movement in 10 and 20 mm, respectively, whereas taurocholate and glycocholate was reported to have no inhibitory effect on ciliary movement even at the end of 60 mm.

Despite the potential for adverse effects on mucociliary clearance attributed to these and other ciliostatic factors, ciliostatic agents nonetheless find use within the methods and compositions of the invention to increase the residence time of mucosally (e.g., intranasally) administered dopamine receptor agonists. In particular, the delivery of dopamine receptor agonists within the invention is significantly enhanced in certain aspects by the coordinate administration or combinatorial formulation of one or more ciliostatic agents that function to reversibly inhibit ciliary activity of mucosal cells, to provide for temporary, reversible increase in the residence time of a mucosally administered dopamine receptor agonist. For use within these aspects of the invention, the foregoing ciliostatic factors, either specific or indirect in their activity, are all candidates for successful employment as ciliostatic agents in appropriate amounts (reflective of concentration, duration and mode of delivery) such that they yield a transient (i.e., reversible) reduction or cessation of mucociliary clearance at a mucosal site of administration of the dopamine receptor agonist, without unacceptable adverse side effects.

Within more detailed aspects, a specific ciliostatic factor is employed, as exemplified by various bacterial ciliostatic factors isolated and characterized in the literature. For example, Hingley, et al. (Infection and Immunity. 51:254-262, 1986, incorporated herein by reference) have recently identified ciliostatic factors from the bacterium Pseudomonas aeruginosa. These are heat-stable factors released by Pseudomonas aeruginosa in culture supernatants that have been shown to inhibit ciliary function in epithelial cell cultures. Exemplary among these cilioinhibitory components are a phenazine derivative, a pyo compound (2-alkyl-4-hydroxyquinolines), and a rhamnolipid (also known as a hemolysin). Inhibitory concentrations of these and other active components were established by quantitative measures of ciliary motility and beat frequency. The pyo compound produced ciliostasis at concentrations of 50 μg/ml and without obvious ultrastructural lesions. The phenazine derivative also inhibited ciliary motility but caused some membrane disruption, although at substantially greater concentrations of 400 μg/ml. Limited exposure of tracheal explants to the rhamnolipid resulted in ciliostasis which was associated with altered ciliary membranes. More extensive exposure to rhamnolipid was associated with removal of dynein arms from axonemes. It is proposed that these and other bacterial ciliostatic factors have evolved to enable P. aeruginosa to more easily and successfully colonize the respiratory tract of mammalian hosts. On this basis, respiratory bacterial are useful pathogens for identification of suitable, specific ciliostatic factors for use within the methods and compositions of the invention.

Several methods are available to measure mucociliary clearance for evaluating the effects and uses of ciliostatic agents within the invention. Nasal mucociliary clearance was initially measured by monitoring the disappearance of visible tracers such as India ink, edicol orange powder, and edicol supra orange. These tracers were followed either by direct observation or with the aid of posterior rhinoscopy or a binocular operating microscope. This method simply measured the time taken by a tracer to travel a definite distance. In more modern techniques, radiolabeled tracers are administered as an aerosol and traced by suitably collimated detectors. Alternatively, particles with a strong taste like saccharin can be placed in the nasal passage and assayed to determine the time before the subject first perceives the taste is used as an indicator of mucociliary clearance.

Additional assays are known in the art for measuring ciliary beat activity. For example, a laser light scattering technique to measure tracheobronchial mucociliary activity is based on mono-chromaticity, coherence, and directionality of laser light. Ciliary motion is measured as intensity fluctuations due to the interference of Doppler-shifted scattered light. The scattered light from moving cilia is detected by a photomultiplier tube and its frequency content analyzed by a signal correlator yielding an autocorrelation function of the detected photocurrents. In this way, both the frequency and synchrony of beating cilia can be measured continuously. Through fiberoptic rhinoscopy, this method also allows the measurement of ciliary activity in the peripheral parts of the nasal passages.

In vitro assays for evaluating ciliostatic activity of formulations within the invention are also available. For example, a commonly used and accepted assay in this context is a rabbit tracheal explant system (Gabridge et al., Pediatr. Res. 1:31-35, 1979; Chandler et al., Infect. Immun. 29:1111-1116, 1980, each incorporated herein by reference). Other assay systems measure the ciliary beat frequency of a single cell or a small number of cells (Kennedy et al., Exp. Cell Res. 135:147-156, 1981; Rutland et al., Lancet ii 564-565, 1980; Verdugo, et al., Pediatr. Res. 13:131-135, 1979, each incorporated herein by reference).

Surface Active Agents and Methods

Within more detailed aspects of the invention, one or more membrane penetration-enhancing agents may be employed within a processing or coordinate administration method or combinatorial formulation of the invention to enhance mucosal delivery of a dopamine receptor agonist. Membrane penetration enhancing agents in this context can be selected from: (i) a surfactant, (ii) a bile salt, (ii) a phospholipid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a clyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, or (x) an inhibitor of cholesterol synthesis; or (xi) any combination of the membrane penetration enhancing agents recited in (i)-(x)

Certain surface-active agents are readily incorporated within the mucosal delivery formulations and methods of the invention as mucosal absorption enhancing agents. These agents, which may be coordinately administered or combinatorially formulated with biologically active agents of the invention, may be selected from a broad assemblage of known surfactants. Surfactants, which generally fall into three classes: (1) nonionic polyoxyethylene ethers; (2) bile salts such as sodium glycocholate (SGC) and deoxycholate (DOC); and (3) derivatives of fusidic acid such as sodium taurodihydrofusidate (STDHF). The mechanisms of action of these various classes of surface active agents typically include solubilization of the biologically active agent. For proteins and peptides which often form aggregates, the surface active properties of these absorption promoters can allow interactions with proteins such that smaller units such as surfactant coated monomers may be more readily maintained in solution. These monomers are presumably more transportable units than aggregates. A second potential mechanism is the protection of the peptide or protein from proteolytic degradation by proteases in the mucosal environment. Both bile salts and some fusidic acid derivatives reportedly inhibit proteolytic degradation of proteins by nasal homogenates at concentrations less than or equivalent to those required to enhance protein absorption. This protease inhibition may be especially important for peptides with short biological half-lives.

The mechanism of absorption enhancement by surface active agents at the mucosal surface may additionally encompass solubilization, rearrangement or other absorption-promoting disturbance of the lipid bilayer of mucosal cell membranes, thus diminishing the barrier to transport across these cells to distant target sites of action (e.g., the systemic circulation or CNS). An alternative mode of action for bile salts may involve the formation of reversed micelles of these compounds in the cell membrane, resulting in a water-filled pore that the active agent(s) can pass through driven by a local concentration gradient. Derivatives of sodium fusidate may act in a similar fashion. Alternatively, surface active agents, alone or complexed with a dopamine receptor agonist or coordinately administered biologically active peptide or protein, may act on the tight junctions between epithelial cells of the mucosa, allowing paracellular transport of the dopamine receptor agonist.

Within exemplary embodiments of the invention, one or more surface active agents is coordinately administered or combinatorially formulated with a dopamine receptor agonist as disclosed herein, in an amount effective to enhance mucosal absorption and/or CNS delivery of the dopamine receptor agonist while not substantially adversely effecting the biological activity of this or other active agent(s) nor causing substantial adverse side effects (e.g., undesirable nasal mucosal irritation resulting in pain, congestion and/or rhinorrhea). Exemplary surface active agents within specific aspects of the invention include, but are not limited to, non-ionic surfactants, such as polysorbates (e.g., polysorbate 80), polyoxyethylene lauryl ether, n-lauryl-β-D-maltopyranoside (LM), cetyl ether, stearyl ether, and nonylphenyl ether, and other surfactants, such as sodium lauryl sulfate, sodium taurochloate, sodium cholate, sodium glycocholate, L-carnitine, and saponin. Also included are different classes of surfactants disclosed elsewhere herein, for example detergents (e.g., Tween 80, Triton X-100) and fatty acid-surfactants (e.g., linoleic acid), which may be used alone or as mixed micellar components. In more detailed aspects of the invention, laureth-9 is employed as a surfactant within the methods and formulations of the invention (see, e.g., Hirai et al., Intl. J. Pharmaceutics 1;173-184, 1981; G. B. Patent specification 1 527 605; and Salzman et al., New Eng. J. Med., April, 1985, 1078-1084, each incorporated herein by reference).

Degradation Enzymes and Inhibitors of Fatty Acid and Cholesterol Synthesis

In related aspects of the invention, dopamine receptor agonists for intranasal administration are formulated or coordinately administered with a penetration enhancing agent selected from a degradation enzyme, or a metabolic stimulatory agent or inhibitor of synthesis of fatty acids, sterols or other selected epithelial barrier components (see, e.g., U.S. Pat. No. 6,190,894). In one embodiment, known enzymes that act on mucosal tissue components to enhance permeability are incorporated in the coordinate administration methods of the instant invention, as processing agents within the multi-processing methods of the invention, or as additives within the combinatorial formulations of the invention. For example, degradative enzymes such as phospholipase, hyaluronidase, neuraminidase, and chondroitinase may be employed to enhance mucosal penetration of dopamine receptor agonists within the methods and compositions of the invention (see, e.g., Squier Brit. J. Dermatol. 111:253-264, 1984; Aungst and Rogers Int. J. Pharm. 53:227-235, 1989, incorporated herein by reference), without causing irreversible damage to the mucosal barrier. In one embodiment, chondroitinase is employed within a method or composition as provided herein to alter glycoprotein or glycolipid constituents of the permeability barrier of the mucosa, thereby enhancing mucosal absorption of the dopamine receptor agonist.

With regard to inhibitors of synthesis of mucosal barrier constituents, it is noted that free fatty acids account for 20-25% of epithelial lipids by weight. Two rate limiting enzymes in the biosynthesis of free fatty acids are acetyl CoA carboxylase and fatty acid synthetase. Through a series of steps, free fatty acids are metabolized into phospholipids. Thus, inhibitors of free fatty acid synthesis and metabolism for use within the methods and compositions of the invention include, but are not limited to, inhibitors of acetyl CoA carboxylase such as 5-tetradecyloxy-2-furancarboxylic acid (TOFA); inhibitors of fatty acid synthetase; inhibitors of phospholipase A such as gomisin A, 2-(p-amylcinnamyl)amino-4-chlorobenzoic acid, bromophenacyl bromide, monoalide, 7,7-dimethyl-5,8-eicosadienoic acid, nicergoline, cepharanthine, nicardipine, quercetin, dibutyryl-cyclic AMP, R-24571, N-oleoylethanolamine, N-(7-nitro-2,1,3-benzoxadiazol-4-yl) phosphostidyl serine, cyclosporine A, topical anesthetics, including dibucaine, prenylamine, retinoids, such as all-trans and 13-cis-retinoic acid, W-7, trifluoperazine, R-24571 (calmidazolium), 1-hexadocyl-3-trifluoroethyl glycero-sn-2-phosphomenthol (MJ33); calcium channel blockers including nicardipine, verapamil, diltiazem, nifedipine, and nimodipine; antimalarials including quinacrine, mepacrine, chloroquine and hydroxychloroquine; beta blockers including propanalol and labetalol; calmodulin antagonists; EGTA; thimersol; glucocorticosteroids including dexamethasone and prednisolone; and nonsteroidal antiinflammatory agents including indomethacin and naproxen.

Each of the foregoing inhibitors of fatty acid synthesis may be coordinately administered or combinatorially formulated with a dopamine receptor agonist of the invention to achieve enhanced epithelial penetration of the dopamine receptor agonist into or across the mucosa. An effective concentration range for the fatty acid synthesis inhibitor for mucosal administration within the invention is generally from about 0.0001% to about 20% by weight of a therapeutic or adjunct formulation, more typically from about 0.01% to about 5%.

Free sterols, primarily cholesterol, account for 20-25% of the epithelial lipids by weight. The rate limiting enzyme in the biosynthesis of cholesterol is 3-hydroxy-3-methylglutaryl (HMG) CoA reductase. Inhibitors of cholesterol synthesis for use within the methods and compositions of the invention include, but are not limited to, competitive inhibitors of (HMG) CoA reductase, such as simvastatin, lovastatin, fluindostatin (fluvastatin), pravastatin, mevastatin, as well as other HMG CoA reductase inhibitors, such as cholesterol oleate, cholesterol sulfate and phosphate, and oxygenated sterols, such as 25-OH— and 26-OH— cholesterol; inhibitors of squalene synthetase; inhibitors of squalene epoxidase; inhibitors of DELTA7 or DELTA24 reductases such as 22,25-diazacholesterol, 20,25-diazacholestenol, AY9944, and triparanol. Each of these sterol synthesis inhibitors may be coordinately administered or combinatorially formulated with a dopamine receptor agonist of the invention to achieve enhanced epithelial penetration of the dopamine receptor agonist into or across the mucosa. An effective concentration range for the sterol inhibitor in a therapeutic or adjunct formulation for intranasal delivery is generally from about 0.0001% to about 20% by weight of the total, more typically from about 0.01% to about 5%.

Nitric Oxide Donor Agents and Methods

Within other related aspects of the invention, a nitric oxide (NO) donor is selected as a membrane penetration-enhancing agents to enhance mucosal delivery of a dopamine receptor agonist within the coordinate administration or processing methods or combinatorial formulations of the invention. Recently, Salzman et al. (Am. J. Physiol. 268:G361-G373, 1995, incorporated herein by reference) reported that NO donors increased the permeability of water-soluble compounds across Caco-2 cell monolayers with neither loss of cell viability nor lactate dehydrogenase (LDH) release. In addition, Utoguchi et al. (Pharm. Res. 15:870-876, 1998, incorporated herein by reference) demonstrated that the rectal absorption of insulin was remarkably enhanced in the presence of NO donors, with attendant low cytotoxicity donors as evaluated by the cell detachment and LDH release studies in Caco-2 cells.

Various NO donors are known in the art and are useful in effective concentrations within the methods and formulations of the invention. Exemplary NO donors include, but are not limited to, nitroglycerine, nitropruside, NOC5 [3-(2-hydroxy-1-(methyl-ethyl)-2-nitrosohydrazino)-1-propanamine], NOC12 [N-ethyl-2-(1-ethyl-hydroxy-2-nitrosohydrazino)-ethanamine], SNAP [S-nitroso-N-acetyl-DL-penicillamine], NORI and NOR4. Efficacy of these and other NO donors for enhancing mucosal and/or CNS delivery of dopamine receptor agonists within the methods and compositions of the invention can be evaluated routinely according to known efficacy and cytotoxicity assay methods (e.g., involving control coadministration of an NO scavenger, such as carboxy-PIIO) as described by Utoguchi et al., Pharm. Res. 15:870-876, 1998 (incorporated herein by reference).

Within the methods and compositions of the invention, an effective amount of a selected NO donor is coordinately administered or combinatorially formulated with a dopamine receptor agonist to enhance the paracellular transport of the dopamine receptor agonist into or through the mucosal epithelium. This pathway is restricted by tight junctions at the apical side of the mucosal epithelial cells. NO donors employed in this context induce a significant increase in the permeability of the mucosa to the biologically active agent, in a manner which is reversible and which evidently involves dilation of the tight junctions between the epithelial cells (e.g., as can be detected by electron microscopy and other methods). This modulation of tight junctional structure is accompanied by an increase in the paracellular permeability as a physiological reaction, with little or no cytotoxic effect on the mucosal epithelium.

Modulation of Epithelial Junction Physiology

As noted above, a primary barrier to paracellular diffusion of molecules and ions across mucosal epithelia (e.g., the nasal mucosa) are cellular junctions known as “tight junctions” (TJ) or “zonula occludens” (ZO). This type of epithelial junction represents one of three distinct morphological elements of the epithelial junctional complex, the other two being the zonula adherens (ZA, or intermediate junction) and the desmosomes. At the tight junction, the plasma membranes are brought into extremely close apposition, but not fused, so as to tightly occlude the extracellular space. Although the degree of permeability of the tight junctions varies in different epithelia, the tight junctions have been reported to be essentially impermeable to molecules with radii of approximately 15 angstroms, unless treated with junctional physiological control agents that stimulate substantial junctional opening. In MDCK cells tight junctions display a characteristic pattern of cation selectivity, which makes them behave as pores with hydrated negative sites.

Consequently, anionic substances may not be able to pass through the nasal epithelium via the paracellular pathway under normal conditions. To increase the permeability of the paracellular pathway in this context, various compounds described elsewhere herein may regulate epithelial junctional physiology by effectuating an ionic increase in the hydrodynamic “pore” size of the mucosal membrane. For example, Na caprylate (C8), Na caprate (C10), Na laurate (C12), salicylates, enamines, and mixed micelles of Na oleate (C18:1) and sodium taurocholate, function within the invention to enhance paracellular permeation via this pathway.

As also noted above, the integrity of epithelial cell tight junctions has long been known to depend on extracellular Ca²⁺. In fact, Ca²⁺ may be regulated within the present invention according to known methods (e.g., using calcium chelators, see Palant et al, Am. J. Physiol., 245:203-212, 1983, incorporated herein by reference) to restore the barrier function of a mucosa following administration of absorption-promoters that impair this function. With respect to regulating junctional physiology, it is believed that the ability to increase epithelial permeability by Ca²⁺ deprivation result indirectly from Ca²⁺ effects on other epithelial junctional components, rather than from direct effects on the tight junction. The most probable junctional element affected is the Ca²⁺-dependent cell adhesion molecule uvomorulin (L-CAM) which belongs to a family of intercellular adhesion molecules that includes placental cadherin and adherin. This protein has been localized to the ZA of the small intestinal epithelium by EM immunocytochemistry, and may act in concert with the actin filaments of the cytoskeleton in a manner that indirectly regulates tight junction permeability.

A variety of additional modulator agents are also useful within the invention that have similarly been shown to alter epithelial junction physiology. Exemlpary agents in this context include nitric oxide (NO) stimulators, chitosan, and chitosan derivatives. Additional agents that can be coordinately administered or combinatorially formulated within the methods and compositions of the invention to regulate junctional physiology elevate intracellular cAMP in the mucosal epithelium (see, e.g., Duffey et al, Nature, 204:451-452, 1981; Bakker et al, Am. J. Physiol., 246:213-217, 1984; Krasney et al, Fed. Proc., 42:1100, 1983; each incorporated herein by reference). Within other aspects of the invention, enhancement in paracellular absorption results not only from expansion in the dimension of the tight junction and the intercellular space, but also from the increase in water influx through that space. This is the case in the promotion of paracellular transport by chelators, such as EDTA, EGTA, citric acid, phytic acid, enamine derivatives, DEEMM; Na caprate, p-aminobenzoic acid, and polyoxyethylated nonionic surfactants. The increase in water flux is Na dependent, as indicated by reduction in its effect by ouabain. This is characteristic of increased water flux in the paracellular pathway when compared with that in the transcellular pathway. Increase in water flux in the transcellular pathway can be induced by diethyl maleate, which reacts with glutathione in the membrane, and by nonsteroidal antiinflammatory drugs, such as indomethacin, diclofenac, and phenylbutazone. Increase in water influx may affect drug absorption within the methods and compositions of the invention by increasing the concentration gradient for penetration, increasing solvent drag, or increasing blood flow in the submucosal vasculature.

Yet additional methods to modulate epithelial permeability within the invention that involve direct or indirect modulation of epithelial junctional physiology include, enhancing Na transport by increasing osmolality of the dosing solution, or by promoting glucose and amino acid transport. In the former context, tight junctions may be induced to open in the presence of a hyperosmotic load, e.g., as previously reported for the rat jejunum after exposure to 600 mOsm mannitol. This led to the appearance of horseradish peroxidase in the intercellular spaces between adjacent absorptive epithelial cells of the jejunal villi. Similarly, the rectal absorption of gentamicin sulfate in rats was enhanced by the use of high ionic strength aqueous formulations.

There is yet another method for use within the invention to promote water flux across mucosal epithelia by indirect regulation ofjunctional physiology that involves energy dependent contractile processes. For example, in the presence of 25 mM glucose fluid flow from the jejunum and upper ileum of the rat was reported to be doubled, as was clearance of creatinine (MW 113, size 3.2 A), PEG 4000 (MW 4,000, size 12.4 A), and insulin (MW 5,500, 14A). Under these conditions, there was a two- to three-fold decrease of resistance with a simultaneous increase of membrane surface (capacitance) and width of the intercellular junctions and lateral spaces (conductance). The equivalent pore radius was estimated to be 50 A. This response was dependent on oxygen tension, indicating the involvement of an energy-dependent contractile process. According to this mechanism, active transport of glucose and amino acids, which is coupled to Na-transport across the intestinal mucosa into the inter-cellular lateral spaces, creates an osmotic force for fluid flow. This in turn triggers contraction of the perijunctional actomyosin ring, resulting in increased paracellular permeability. Involvement of actin filaments in this process is indicated by gradual increase in paracellular permeability upon exposure to cytochalasins, drugs that disrupt actin filaments which interact directly with the ZO and ZA. Besides cytochalasins, phorbol esters, through stimulating protein kinase C (a Ca²⁺ phospholipid-dependent enzyme), are also useful within the invention to induce opening of tight junctions. Activation of PKC by phorbol esters increases paracellular permeability both in kidney and intestinal epithelial cell lines (Ellis et al, Am. J. Phvsiol. 263:293-300, 1992; Stenson et al, C. Am. J. Physiol. 265:955-962, 1993, each incorporated herein by reference).

Within more detailed aspects of the invention, junctional physiology is modulated by specific agents that target particular components of epithelial junctional complexes for physiological modulation. Broadly embraced within these aspects of the invention are specific binding or blocking agents, such as antibodies, antibody fragments, peptides, peptide mimetics, bacterial toxins and other agents that serve as agonists or antagonists to the normal regulatory function of junctional component molecules, particularly junctional protein complexes, signal-transduction factors, ligands and receptors. Among these particular components, two polypeptides from ZO junctions, designated ZO-1 and ZO-2 exist as a heterodimer in a detergent-stable complex with an uncharacterized 130 kD protein ZO-3 (Gumbiner et al, Proc. Natl. Acad. Sci., USA, 88:3460-3464, 1991; U.S. Pat. Nos. 5,945,510; 5,948,629; 5,912,323; 5,864,014; 5,827,534; 5,665,389, each incorporated herein by reference). Most immunoelectron microscopic studies have localized ZO-1 to a position most closely proximate to membrane contacts between epithelial cells (Stevenson et al, Molec. Cell Biochem., 83:129-145, 1988, incorporated herein by reference). Two other proteins, cingulin (Citi et al, Nature, 333:272-275, 1988, incorporated herein by reference) and the 7H6 antigen (Zhong et al, J. Cell Biol., 120:477-483, 1993, incorporated herein by reference) are localized further from the membrane and have not yet been cloned. Rab 13, a small GTP binding protein has also recently been localized to a junctional site (Zahraoui et al, J. Cell Biol., 124:101-115, 1994, incorporated herein by reference). Other small GTP-binding proteins are known to regulate the cortical actin cytoskeleton. For example, rho regulates actin-membrane attachment in focal contacts (Ridley et al, Cell, 70:389-399, 1992, incorporated herein by reference), and rac regulates growth factor-induced membrane ruffling (Ridley et al, Cell, 70:401-410, 1992, incorporated herein by reference). Based on structure-function analyses of other known proteins associated with cell junctions, focal contacts, and adherens junctions, it is projected that tight junction-associated plaque proteins are involved in transducing signals in both directions across the cell membrane, and in regulating links to the cortical actin cytoskeleton that indirectly regulate membrane permeation. (Guan et al, Nature, 358:690-692 (1992; Tsukita et al, J. Cell Biol., 123:1049-1053, 1993, each incorporated herein by reference).

Among the tight junctional regulatory components that serve as useful targets for physiological modulation within the methods and compositions of the invention, the ZO1-ZO2 heterodimeric complex has shown itself amenable to physiological regulation by exogenous agents that can readily and effectively alter paracellular permeability in mucosal epithelia. On such agent which has been extensively studied is the bacterial toxin from Vibrio cholerae known as the “zonula occludens toxin” (ZOT). This toxin mediates increased intestinal mucosal permeability and causes disease symptoms including diarrhea in infected subjects (Fasano et al, Proc. Nat. Acad. Sci., USA, 8:5242-5246, 1991; Johnson et al, J. Clin. Microb., 31/3:732-733, 1993; and Karasawa et al, FEBS Let., 106:143-146, 1993, each incorporated herein by reference). When tested on rabbit ileal mucosa, ZOT increased the intestinal permeability by modulating the structure of intercellular tight junctions. More recently, it has been found that ZOT is capable of reversibly opening tight junctions in the intestinal mucosa (see, e.g., WO 96/37196; U.S. Pat. Nos. 5,945,510; 5,948,629; 5,912,323; 5,864,014; 5,827,534; 5,665,389, each incorporated herein by reference). It has also been reported that ZOT is capable of reversibly opening tight junctions in the nasal mucosa (U.S. Pat. No. 5,908,825, incorporated herein by reference).

Within the methods and compositions of the invention, ZOT, as well as various analogs and mimetics of ZOT that function as agonists or antagonists of ZOT activity, are useful for enhancing mucosal delivery of dopamine receptor agonists—by increasing paracellular absorption into and across the mucosal epithelium. In this context, ZOT typically acts by causing a structural reorganization of tight junctions marked by altered localization of the junctional protein ZO1. Within these aspects of the invention, ZOT is coordinately administered or combinatorially formulated with the biologically active agent in an effective amount to yield significantly enhanced absorption of the active agent, by reversibly increasing mucosal permeability without substantial adverse side effects Suitable methods for determining ZOT biological activity may be selected from a variety of known assays, e.g., involving detection of a decrease in tissue or cell culture resistance (Rt) using Ussing chambers (e.g., as described by Fasano et al, Proc. Natl. Acad. Sci., USA, 8:5242-5246, 1991, incorporated herein by reference), assaying for a decrease of tissue resistance (Rt) of intestinal epithelial cell monolayers in Ussing chambers as described in Example 3 below; or directly assaying enhancement of absorption of a therapeutic agent across a mucosal surface in vivo.

In addition to ZOT, various other tight junction modulatory agents can be employed within the methods and compositions of the invention that mimic the activity of ZOT by reversibly increasing mucosal epithelial paracellular permeability. These include specific binding or blocking agents, such as antibodies, antibody fragments, peptides, peptide mimetics, bacterial toxins and other agents that serve as agonists or antagonists of ZOT activity, or which otherwise alter physiology of the ZO1-ZO2 complex (e.g., by blocking dimerization). Naturally, these additional regulatory agents include peptide analogs, including site-directed mutant variants, of the native ZOT protein, as well as truncated active forms of the protein and peptide mimetics that model functional domains or active sites of the native protein. In addition, these agents include a native mammalian protein “zonulin”, which has been proposed to be an endogenous regulator of tight junctional physiology similar in both structural and functional aspects to ZOT (see, e.g., WO 96/37196; WO 00/07609; U.S. Pat. Nos. 5,945,510; 5,948,629; 5,912,323; 5,864,014; 5,827,534; 5,665,389, each incorporated herein by reference), which therefore suggests that ZOT is a convergent evolutionary development of Vibrio cholerae patterned after the endogenous mammalian zonulin regulatory mechanism to facilitate host entry. Both zonulin and ZOT are proposed to bind a specific membrane receptor, designated “ZOT receptor” (see, e.g., U.S. Pat. No. 5,864,014; 5,912,323; and 5,948,629, each incorporated herein by reference), which can be used within the invention to screen for additional agonists and antagonists to ZOT and zonulin activity for regulation of tight junctional physiology. In this context, structure-function analysis of the ZOT receptor, and comparisons between ZOT and zonulin, will guide production and selection of specific binding or blocking agents, (e.g., antibodies, antibody fragments, peptides, peptide mimetics, additional bacterial toxins and other agents) to serve as ZOT or zonulin agonists or antagonists, for example with respect to ZOT or zonulin binding or activation of the ZOT receptor, to regulate tight junctional physiology within the methods and compositions of the invention.

Yet additional methods and agents for modulating junctional physiology and enhancing mucosal and/or CNS delivery of dopamine receptor agonists within the invention are directed to junctional adhesion molecules (JAMs). JAMs are endogenously regulated by immune and inflammatory effector cells, including lymphocytes and macrophages, which are able to transit epithelial barriers, including epithelial junctions, presumptively by expanding junctional “pores” to as much as 500 nm or more in passable diameter. This phenomenon is illustrated in a report by Alpar et al., J. Drug. Target. 2:147-9, 1994 documenting nasal mucosal absorption of labeled microspheres. Specifically, this report demonstrates uptake by nasal epithelial tissue of fluorescent polystyrene latex microparticles of diameter 0.8 micron in rats after single intranasal dosing. At intervals following administration, particles were observed in the blood compartment. Peak concentration of particles occurred in normal animals at 10 min. At 24 h some particles were still present in these animals' circulation. Throughout the sampling, tracheotomised animals demonstrated a steady state presence of particles. These results show that the uptake and translocation of solid particles takes place through the nasal epithelial lining as it does through gut epithelia, possibly through the nasal associated lymphatic tissue.

Vasodilator Agents and Methods

Yet another class of absorption-promoting agents that show beneficial utility within the coordinate administration and processing methods and combinatorial formulations of the invention are vasoactive compounds, more specifically vasodilators. These compounds function within the invention to modulate the structure and physiology of the submucosal vasculature, increasing the transport rate of dopamine receptor agonists and other biologically active agents from the base of the mucosal epithelium into the local (e.g., nasopharyngeal or cerebral) or systemic circulation.

Vasodilator agents for use within the invention typically cause submucosal blood vessel relaxation by either a decrease in cytoplasmic calcium, an increase in nitric oxide (NO) or by inhibiting myosin light chain kinase. They are generally divided into 9 classes: calcium antagonists, potassium channel openers, ACE inhibitors, angiotensin-II receptor antagonists, α-adrenergic and imidazole receptor antagonists, β1-adrenergic agonists, phosphodiesterase inhibitors, eicosanoids and NO donors.

Despite chemical differences, the pharmacokinetic properties of calcium antagonists are similar. Absorption into the systemic circulation is high, and these agents therefore undero considerable first-pass metabolism by the liver, resulting in individual variation in pharmacokinetics. Except for the newer drugs of the dihydropyridine type (amlodipine, felodipine, isradipine, nilvadipine, nisoldipine and nitrendipine), the half-life of calcium antagonists is short. Therefore, to maintain an effective drug concentration for many of these may require delivery by multiple dosing, or controlled release formulations, as described elsewhere herein. Delivery enhancement using the potassium channel opener minoxidil may also be limited in manner and level of administration due to potential adverse side effects.

ACE inhibitors, which prevent conversion of angiotensin-I to angiotensin-II, and are most effective when renin production is increased. Since ACE is identical to kininase-II, which inactivates the potent endogenous vasodilator bradykinin, ACE inhibition causes a reduction in bradykinin degradation. ACE inhibitors provide the added advantage of cardioprotective and cardioreparative effects, by preventing and reversing cardiac fibrosis and ventricular hypertrophy in animal models. The predominant elimination pathway of most ACE inhibitors is via renal excretion. Therefore, renal impairment is associated with reduced elimination and a dosage reduction of 25 to 50% is recommended in patients with moderate to severe renal impairment.

Separating angiotensin-II inhibition from bradykinin potentiation has been the goal in developing angiotensin-II receptor antagonists. The incidence of adverse effects of such an agent, losartan, is comparable to that encountered with placebo treatment, and the troublesome cough associated with ACE inhibitors is absent.

With regard to NO donors, these compounds are particularly useful within the invention for their additional effects on mucosal permeability (see above). In addition to the above-noted NO donors, complexes of NO with nucleophiles called NO/nucleophiles, or NONOates, spontaneously and nonenzymatically release NO when dissolved in aqueous solution at physiologic pH (Cornfield et al., J. Lab. Clin. Med., 134(4):419-425, 1999, incorporated herein by reference). In contrast, nitro vasodilators such as nitroglycerin require specific enzyme activity for NO release. NONOates release NO with a defined stoichiometry and at predictable rates ranging from <3 minutes for diethylamine/NO to approximately 20 hours for diethylenetriamine/NO (DETANO).

Within certain methods and compositions of the invention, a selected vasodilator agent is coordinately administered (e.g., systemically or mucosally, simultaneously or in combinatorially effective temporal association) or combinatorially formulated with a dopamine receptor agonist in an amount effective to enhance mucosal absorption of the dopamine receptor agonist to reach a target site for activity (e.g., the systemic circulation or CNS).

Selective Transport-Enhancing Agents and Methods

Within certain aspects of the invention, mucosal delivery of dopamine receptor agonists is enhanced by methods and agents that target selective transport mechanisms and promote endo- or transcytocis of the dopamine receptor agonist and, optionally, other macromoloecular drugs, carriers and delivery enhancers. In this regard, the compositions and delivery methods of the invention optionally incorporate a selective transport-enhancing agent that facilitates transport of the dopamine receptor agonist through transport barriers into the mucosal tissues and/or to other target(s), such as the circulatory system or CNS.

Exemplary selective transport-enhancing agents for use within this aspect of the invention include, but are not limited to, glycosides, sugar containing molecules, and binding agents such as lectin binding agents which are known to interact specifically with epithelial transport barrier components (see, e.g., Goldstein et al., Annu. Rev. Cell. Biol. 1:1-39, 1985, incorporated herein by reference). For example, specific “bioadhesive” ligands, including various plant and bacterial lectins, chitosans and modified chitosans such as poly-GuD, and other agents which bind to cell surface sugar moieties by receptor-mediated interactions can be employed as carriers or conjugated transport mediators for enhancing mucosal delivery of dopamine receptor agonists within the invention. For example, certain bioadhesive ligands mediate transmission of biological signals to mucosal epithelial target cells that trigger selective uptake of the adhesive ligand by specialized cellular transport processes (endocytosis or transcytosis). These transport mediators can therefore be employed as a “carrier system” or conjugate partner to stimulate or mediate selective uptake of dopamine receptor agonists into and/or through mucosal epithelia. These and other selective transport-enhancing agents significantly enhance mucosal delivery of dopamine receptor agonists and other macromolecular biopharmaceuticals (particularly peptides, proteins, oligonucleotides and polynucleotide vectors) within the invention. To utilize these transport-enhancing agents, general carrier formulation and/or conjugation methods as described elsewhere herein are used to coordinately administer a selective transport enhancer (e.g., a receptor-specific ligand) and a dopamine receptor agonist to a mucosal surface, whereby the transport-enhancing agent is effective to trigger or mediate enhanced endo- or transcytosis of the dopamine receptor agonist into or across the mucosal epithelium or another target cell or tissue.

Lectins are plant proteins that bind to specific sugars found on the surface of glycoproteins and glycolipids of eukaryotic cells. Such binding may result in specific haemagglutinating activity. Since lectins are relatively heat stable, they are abundant in the human diet (e.g., cereals, beans and other seeds). Concentrated solutions of lectins have a ‘mucotractive’ effect due to irritation of the gut wall, which explains why so-called ‘high fiber foods’ (rich in lectins) are thought to be responsible for stimulating bowel motility. Various studies have demonstrated rapid receptor mediated endocytocis (RME) of lectins and lectin conjugates (e.g., concanavalin A conjugated with colloidal gold particles) across mucosal surfaces. Other studies report that the uptake mechanisms for lectins can be utilized for intestinal drug targeting in vivo.

In addition to plant lectins, microbial adhesion and invasion factors provide a rich source of candidates for use as adhesive/selective transport carriers within the methods and compositions of the invention (see, e.g., Lehr, Crit. Rev. Therap. Drug Carrier Syst. 11:177-218, 1995; Swann, P A, Pharmaceutical Research 15:826-832, 1998, each incorporated herein by reference). Two components are necessary for bacterial adherence processes, a bacterial ‘adhesin’ (adherence or colonization factor) and a receptor on the host cell surface. Bacteria causing mucosal infections need to penetrate the mucus layer before attaching themselves to the epithelial surface. This attachment is usually mediated by bacterial fimbriae or pilus structures, although other cell surface components may also take part in the process. Adherent bacteria colonize mucosal epithelia by multiplication and initiation of a series of biochemical reactions inside the target cell through signal transduction mechanisms (with or without the help of toxins). Associated with these invasive mechanisms, a wide diversity of bioadhesive proteins (e.g., invasin, internalin) originally produced by various bacteria and viruses are known. These allow for extracellular attachment of such microorganisms with an impressive selectivity for host species and even particular target tissues. Signals transmitted by such receptor-ligand interactions trigger the transport of intact, living microorganisms into, and eventually through, epithelial cells by endo- and transcytotic processes. Such naturally occurring phenomena may be harnessed (e.g., by complexing biologically active agents with adhesins) according to the teachings herein for enhanced delivery of dopamine receptor agonists across mucosal (e.g., nasal mucosal) epithelia to designated target sites of drug action (e.g., the CNS). One advantage of this strategy is that the selective carrier partners thus employed are substrate-specific, leaving the natural barrier function of tight epithelial tissues intact against other solutes (see, e.g., Lehr, Drug Absorption Enhancement, pp. 325-362, de Boer, Ed., Harwood Academic Publishers, 1994, incorporated herein by reference).

Various bacterial and plant toxins that bind epithelial surfaces in a specific, lectin-like manner are also useful within the methods and compositions of the invention. For example, diptheria toxin (DT) enters host cells rapidly by RME. Likewise, the B subunit of the E. coli heat labile toxin binds to the brush border of intestinal epithelial cells in a highly specific, lectin-like manner. Uptake of this toxin and transcytosis to the basolateral side of the enterocytes has been reported in vivo and in vitro. Fisher and co-workers expressed the transmembrane domain of diphtheria toxin in E. coli as a maltose-binding fusion protein and coupled it chemically to high-Mw poly-L-lysine. The resulting complex has been successfully used to mediate the internalization of a reporter gene in vitro. In addition to these examples, Staphylococcus aureus produces a set of proteins (e.g., staphylococcal enterotoxin A (SEA), SEB, toxic shock syndrome toxin 1 (TSST-1) which act both as superantigens and toxins.

Various plant toxins, mostly ribosome-inactivating proteins (RIPs), have been identified that bind to any mammalian cell surface expressing galactose units and are subsequently internalized by RME. Toxins such as nigrin b, α-sarcin, ricin and saporin, viscumin, and modeccin are highly toxic upon oral administration (i.e., are rapidly internalized). Therefore, modified, less toxic subunits of these compound will be useful within the invention to facilitate the mucosal delivery of dopamine receptor agonists.

Viral haemagglutinins comprise another type of transport agent to facilitate mucosal delivery of dop amine receptor agonists within the methods and compositions of the invention. The initial step in many viral infections is the binding of surface proteins (haemagglutinins) to mucosal cells. These binding proteins have been identified for most viruses, including rotaviruses, varicella zoster virus, semliki forest virus, adenoviruses, potato leafroll virus, and reovirus.

A variety of endogenous, selective transport-mediating factors are also available for use within the invention. Mammalian cells have developed an assortment of mechanisms to facilitate the internalization of specific substrates and target these to defined compartments. Collectively, these processes of membrane deformations are termed ‘endocytosis’ and comprise phagocytosis, pinocytosis, receptor-mediated endocytosis (clathrin-mediated RME), and potocytosis (non-clathrin-mediated RME). RME is a highly specific cellular biologic process by which, as its name implies, various ligands bind to cell surface receptors and are subsequently internalized and trafficked within the cell. In many cells the process of endocytosis is so active that the entire membrane surface is internalized and replaced in less than a half hour.

RME is initiated when specific ligands bind externally oriented membrane receptors. Binding occurs quickly and is followed by membrane invagination until an internal vesicle forms within the cell (the early endosome, “receptosome”, or CURL (compartment of uncoupling receptor and ligand). Localized membrane proteins, lipids and extracellular solutes are also internalized during this process. When the ligand binds to its specific receptor, the ligand-receptor complex accumulates in coated pits. Coated pits are areas of the membrane with high concentration of endocellular clathrin subunits. The assembly of clathrin molecules on the coated pit is believed to aid the invagination process. Specialized coat proteins called adaptins, trap specific membrane receptors which move laterally through the membrane in the coated pit area by binding to a signal sequence (Tyr-X-Arg-Phe, where X=any amino acid) at the endocellular carboxy terminus of the receptor. This process ensures that the correct receptors are concentrated in the coated pit areas and minimizes the amount of extracellular fluid that is taken up in the cell.

Following the internalization process, the clathrin coat is lost through the help of chaperone proteins, and proton pumps lower the endosomal pH to approximately 5.5, which causes dissociation of the receptor-ligand complex. CURL serves as a compartment to segregate the recycling receptor (e.g. transferrin) from receptor involved in transcytosis (e.g. transcoba-lamin). Endosomes may then move randomly or by saltatory motion along the microtubules until they reach the trans-Golgi reticulum where they are believed to fuse with Golgi components or other membranous compartments and convert into tubulovesicular complexes and late endosomes or multivesicular bodies. The fate of the receptor and ligand are determined in these sorting vesicles. Some ligands and receptors are returned to the cell surface where the ligand is released into the extracellular milieu and the receptor is recycled. Alternatively, the ligand is directed to lysosomes for destruction while the receptor is recycled to the cell membrane. The endocytotic recycling pathways of polarized epithelial cells are generally more complex than in non-polarized cells. In these enterocytes a common recycling compartment exists that receives molecules from both apical and basolateral membranes and is able to correctly return them to the appropriate membrane or membrane recycling compartment.

Current understanding of RME receptor structure and related structure-function relationships has been significantly enhanced by the cloning of mRNA sequences coding for endocytotic receptors. Most RME receptors share principal structural features, such as an extracellular ligand binding site, a single hydrophobic transmembrane domain (unless the receptor is expressed as a dimer), and a cytoplasmic tail encoding endocytosis and other functional signals. Two classes of receptors are proposed based on their orientation in the cell membrane; the amino terminus of Type I receptors is located on the extracellular side of the membrane, whereas Type II receptors have this same protein tail in the intracellular milieu.

As noted above, potocytosis, or non-clathrin coated endocytosis, takes place through caveolae, which are uniform omega- or flask-shaped membrane invaginations 50-80 nm in diameter. This process was first described as the internalization mechanism of the vitamin folic acid. Morphological studies have implicated caveolae in i) the transcytosis of macromolecules across endothelial cells; (ii) the uptake of small molecules via potocytosis involving GPI-linked receptor molecules and an unknown anion transport protein; iii) interactions with the actin-based cytoskeleton; and (iv) the compartmentalization of certain signaling molecules involved in signal transduction, including G-protein coupled receptors. Caveolae are characterized by the presence of an integral 22-kDa membrane protein termed VIP21-caveolin, which coats the cytoplasmic surface of the membrane. From a drug delivery standpoint, the advantage of potocytosis pathways over clathrin-coated RME pathways lies in the absence of the pH lowering step, which circumvents the endosomal/lysosomal pathway. This pathway for selective transporter-mediated delivery of biologically active agents is therefore particularly effective for enhanced delivery of pH-sensitive macromolecules.

Exemplary among potocytotic transport carriers mechanisms for use within the invention is the folate carrier system, which mediates transport of the vitamin folic acid (FA) into target cells via specific binding to the folate receptor (FR) (see, e.g., Reddy et al., Crit. Rev. Ther. Drug Car. Syst. 15:587-627, 1998, incorporated herein by reference). The cellular uptake of free folic acid is mediated by the folate receptor and/or the reduced folate carrier. The folate receptor is a glycosylphosphatidylinositol (GPI)-anchored 38 kDa glycoprotein clustered in caveolae mediating cell transport by potocytosis. While the expression of the reduced folate carrier is ubiquitously distributed in eukaryotic cells, the folate receptor is principally overexpressed in human tumors. Two homologous isoforms (α and β) of the receptor have been identified in humans. The α-isoform is found to be frequently overexprssed in epithelial tumors, whereas the β-form is often found in non-epithelial lineage tumors. Consequently, this receptor system has been used in drug-targeting approaches to cancer cells, but also in protein delivery, gene delivery, and targeting of antisense oligonucleotides to a variety of cell types.

Folate-drug conjugates are well suited for use within the mucosal delivery methods of the invention, because they allow penetration of target cells exclusively via FR-mediated endocytosis. When FA is covalently linked, for example, via its γ-carboxyl to a biologically active agent, FR binding affinity (KD˜10⁻¹⁰M) is not significantly compromised, and endocytosis proceeds relatively unhindered, promoting uptake of the attached active agent by the FR-expressing cell. Because FRs are significantly overexpressed on a large fraction of human cancer cells (e.g., ovarian, lung, breast, endometrial, renal, colon, and cancers of mycloid hematopoietic cells), this methodology allows for selective delivery of a wide range of therapeutic as well as diagnostic agents to tumors. Folate-mediated tumor targeting has been exploited to date for delivery of the following classes of molecules and molecular complexes that find use within the invention: (i) protein toxins, (ii) low-molecular-weight chemotherapeutic agents, (iii) radioimaging agents, (iv) MRI contrast agents, (v) radio-therapeutic agents, (vi) liposomes with entrapped drugs, (vii) genes, (viii) antisense oligonucleotides, (ix) ribozymes, and (x) immunotherapeutic agents (see, e.g., Swann, P A, Pharmaceutical Research 15:826-832, 1998, incorporated herein by reference). In virtually all cases, in vitro studies demonstrate a significant improvement in potency and/or cancer-cell specificity over the nontargeted form of the same pharmaceutical agent.

In addition to the folate receptor pathway, a variety of additional methods to stimulate transcytosis within the invention are directed to the transferrin receptor pathway, and the riboflavin receptor pathway. In one aspect, conjugation of a biologically active agent to riboflavin can effectuate RME-mediated uptake. Yet additional embodiments of the invention utilize vitamin B12 (cobalamin) as a specialized transport protein (e.g., conjugation partner) to facilitate entry of biologically active agents into target cells. This system has been shown to be useful for enhancing intestinal uptake of luteinizing hormone releasing factor (LHRH)-analogs, granulocyte colony stimulating factor, erythropoietin, a-interferon, and the LHRH-antagonist ANTIDE.

Still other embodiments of the invention utilize transferrin as carrier or stimulant of RME of mucosally delivered dopamine receptor agonists. Transferrin, an 80 kDa iron-transporting glycoprotein, is efficiently taken up into cells by RME. Transferrin receptors are found on the surface of most proliferating cells, in elevated numbers on erythroblasts and on many kinds of tumors. According to current knowledge of intestinal iron absorption, transferrin is excreted into the intestinal lumen in the form of apotransferrin and is highly stable to attacks from intestinal peptidases. In most cells, diferric transferrin binds to transferrin receptor (TfR), a dimeric transmembrane glycoprotein of 180 kDa, and the ligand-receptor complex is endocytosed within clathrin-coated vesicles. After acidification of these vesicles, iron dissociates from the transferrin/TfR complex and enters the cytoplasm, where it is bound by ferritin (Fn). Recently, it was reported that insulin covalently coupled to transferrin, was transported across Caco-2 cell monolayers by RME. More recently, it has been reported that oral administration of this complex to streptozotocin-induced diabetic mice significantly reduced plasma glucose levels (28%) which was further potentiated by BFA pretreatment (˜41%). The transcytosis of transferrin (Tf) and transferrin conjugates is enhanced in the presence of Brefeldin A (BFA), a fungal metabolite. In other studies, it is reported that BFA treatment rapidly increased apical endocytosis of both ricin and HRP in MDCK cells. Thus, BFA and other agents that stimulate receptor-mediated transport can be employed within the methods of the invention as coordinately administered or combinatorially agents to enhance receptor-mediated transport of dopamine receptor agonists.

Immunoglobulin transport mechanisms provide yet additional endogenous pathways and reagents for incorporation within the mucosal delivery methods and compositions of the invention. Receptor-mediated transcytosis of immunoglobulin G (IgG) across the neonatal small intestine serves to convey passive immunity to many newborn mammals. In rats, IgG in milk selectively binds to neonatal Fc receptors (FcRn) expressed on the surface of the proximal small intestinal enterocytes during the first three weeks after birth. FcRn binds IgG in a pH-dependent manner, with binding occurring at the luminal pH (approx. 6-6.5) of the jejunum and release at the pH of plasma (approx. 7.4). The Fc receptor resembles the major histocompatibility complex (MHC) class I antigens in that it consists of two subunits, a transmembrane glycoprotein (gp50) in association with β2-microglobulin. In mature absorptive cells both subunits are colocalized in each of the membrane compartments that mediate transcytosis of IgG. IgG administered in situ apparently causes both subunits to concentrate within endocytic pits of the apical plasma membrane, suggesting that ligand causes redistribution of receptors at this site. These results support a model for transport in which IgG is transferred across the cell as a complex with both subunits.

Within the methods and compositions of the present invention, IgG and other immune system-related carriers (including polyclonal and monoclonal antibodies and various fragments thereof) can be combinatorially formulated or otherwise coordinately administered with dopamine receptor agonists and, optionally, other biologically active agents, to provide for targeted delivery, typically by receptor-mediated transport, of the dopamine receptor agonist. For example, the dopamine receptor agonist may be covalently linked to the IgG or other immunological active agent or, alternatively, formulated in liposomes or other carrier vehicle which is in turn modified (e.g., coated or covalently linked) to incorporate IgG or other immunological transport enhancer. In certain embodiments, polymeric IgA and/or IgM transport agents are employed, which bind to the polymeric immunoglobulin receptors (pIgRs) of target epithelial cells. Within these methods, expression of pIgR can be enhanced by cytokines.

Within more detailed aspects of the invention, antibodies and other immunological transport agents may be themselves modified for enhanced mucosal delivery, for example, as described in detail elsewhere herein, antibodies may be more effectively administered within the methods and compositions of the invention by charge modifying techniques. In one such aspect, an antibody drug delivery strategy involving antibody cationization is utilized that facilitates both trans-endothelial migration and target cell endocytosis (see, e.g., Pardridge, et al., JPET 286:548-544, 1998, incorporated herein by reference). In this strategy, the pI of the antibody is increased by converting surface carboxyl groups of the protein to extended primary amino groups. These cationized homologous proteins have no measurable tissue toxicity and have minimal immunogenicity. In addition, monoclonal antibodies may be cationized with retention of affinity for the target protein.

Additional selective transport-enhancing agents for use within the invention comprise whole bacteria and viruses, including genetically engineered bacteria and viruses, as well as components of such bacteria and viruses. Aside from conventional gene delivery vectors (e.g., adenovirus), this aspect of the invention includes the use of bacterial ghosts and subunit constructs, e.g., as described by Huter et al., Journal of Controlled Release 61:51-63, 1999 (incorporated herein by reference). Bacterial ghosts are non-denatured bacterial cell envelopes, for example as produced by the controlled expression of the plasmid-encoded lysis gene E of bacteriophage PhiX174 in gram-negative bacteria. Protein E-specific lysis does not cause any physical or chemical denaturation to bacterial surface structures, and bacterial ghosts are therefore useful in development of inactivated whole-cell vaccines. Ghosts produced from Actinobacillus pleuropneumoniae, Pasteurella haemolytica and Salmonella sp. have proved successful in vaccination experiments. Recombinant bacterial ghosts can be created by the expression of foreign genes fused to a membrane-targeting sequence, and thus can carry foreign therapeutic proteins anchored in their envelope. The fact that bacterial ghosts preserve a native cell wall, including bioadhesive structures like fimbriae of their living counterparts, makes them suitable for the attachment to specific target tissues such as nasal mucosal surfaces. Bacterial ghosts have been shown to be readily taken up by macrophages, thus adhesion of ghosts to specific tissues can be followed by uptake through phagocytes.

In view of the foregoing, a wide variety of ligands involved in receptor-mediated transport mechanisms are known in the art and can be variously employed within the methods and compositions of the invention (e.g., as conjugate partners or coordinately administered mediators) to enhance receptor-mediated transport of dopamine receptor agonists. Generally, these ligands include hormones and growth factors, bacterial adhesins and toxins, lectins, metal ions and their carriers, vitamins, immunoglobulins, whole viruses and bacteria or selected components thereof. Exemplary ligands among these classes include, for example, calcitonin, prolactin, epidermal growth factor, glucagon, growth hormone, estrogen, lutenizing hormone, platelet derived growth factor, thyroid stimulating hormone, thyroid hormone, cholera toxin, diptheria toxin, E. coli heat labile toxin, Staphylococcal enterotoxins A and B, ricin, saporin, modeccin, nigrin, sarcin, concanavalin A, transcobalantin, catecholamines, transferrin, folate, riboflavin, vitamin B1, low density lipoprotein, maternal IgO, polymeric IgA, adenovirus, vesicular stomatitis virus, Rous sarcoma virus, V. cholerae, Kiebsiella strains, Serratia strains, parainfluenza virus, respiratory syncytial virus, Varicella zoster, and Enterobacter strains (see, e.g., Swann, P A, Pharmaceutical Research 15:826-832, 1998, incorporated herein by reference).

In certain additional embodiments of the invention, membrane-permeable peptides (e.g., “arginine rich peptides”) are employed to facilitate delivery of dopamine receptor agonists. While the mechanism of action of these peptides remains to be fully elucidated, they provide useful delivery enhancing adjuncts for use within the intranasal delivery compositions and methods herein. In one example, a basic peptide derived from human immunodeficiency virus (HIV)-1 Tat protein (e.g., residues 48-60) has been reported to translocate effectively through cell membranes and accumulate in the nucleus, a characteristic which can be utilized for the delivery of exogenous proteins into cells. The sequence of Tat (GRKKRRQRRRPPQ) comprises a highly basic and hydrophilic peptide, which contains 6 arginine and 2 lysine residues in its 13 amino acid residues. Various other arginine-rich peptides have been identified which have a translocation activity very similar to Tat-(48-60). These include such peptides as the D-amino acid- and arginine-substituted Tat-(48-60), the RNA-binding peptides derived from virus proteins, such as HIV-1 Rev, and flock house virus coat proteins, and the DNA binding segments of leucine zipper proteins, such as cancer-related proteins c-Fos and c-Jun, and the yeast transcription factor GCN4 (see, e.g., Futaki et al., Journal Biological Chemistry 276:5836-5840, 2000, incorporated herein by reference). These peptides reportedly have several arginine residues marking their only identified common structural characteristic, suggesting a common internalization mechanism ubiquitous to arginine-rich peptides, which is not explained by typical endocytosis. Using (Arg)ₙ (n=4-16) peptides, Futaki et al. teach optimization of arginine residues (n 8) for efficient translocation. Recently, methods have been developed for the delivery of exogenous proteins into living cells with the help of arginine rich membrane-permeable carrier peptides such as HIV-I Tat- and Antennapedia-(see, Futaki et al., supra, and references cited therein, incorporated herein by reference). By genetically or chemically hybridizing these carrier peptides with biologically active agents as described herein, additional methods and compositions are provided within the invention to enhance mucosal delivery of dopamine receptor agonists. These methods are generally exemplified by a reported Tat-β-galactosidasefusion protein which has a molecular mass as high as 120 kDa. Intraperitoneal injection of this protein resulted in delivery of the protein with β-galactosidase activity to various tissues in mice, including the brain.

Polymeric Delivery Vehicles and Methods

Within certain aspects of the invention, dopamine receptor agonists, and optionally, other biologically active agents and delivery-enhancing agents as described above, are incorporated within a mucosally (e.g., nasally) administered formulation which comprises a biocompatible polymer functioning as a carrier or base. Such polymer carriers include polymeric powders, matrices or microparticulate delivery vehicles, among other polymer forms. The polymer can be of plant, animal, or synthetic origin. Often the polymer is crosslinked. Additionally, in these delivery systems the biologically active agent can be functionalized in a manner where it can be covalently bound to the polymer and rendered inseparable from the polymer by simple washing. In other embodiments, the polymer is chemically modified with an inhibitor of enzymes or other agents which may degrade or inactivate the dopamine receptor agonist or other biologically active or delivery enhancing agent(s). In certain formulations, the polymer is a partially or completely water insoluble but water swellable polymer, e.g, a hydrogel. Polymers useful in this aspect of the invention are desirably water interactive and/or hydrophilic in nature to absorb significant quantities of water, and they often form hydrogels when placed in contact with water or aqueous media for a period of time sufficient to reach equilibrium with water. In more detailed embodiments, the polymer is a hydrogel which, when placed in contact with excess water, absorbs at least two times its weight of water at equilibrium when exposed to water at room temperature (see, e.g., U.S. Pat. No. 6,004,583, incorporated herein by reference).

Drug delivery systems based on biodegradable polymers are preferred in many biomedical applications because such systems are broken down either by hydrolysis or by enzymatic reaction into non-toxic molecules. The rate of degradation is controlled by manipulating the composition of the biodegradable polymer matrix. These types of systems can therefore be employed in certain settings for long-term release of biologically active agents. Biodegradable polymers such as poly(glycolic acid) (PGA), poly-(lactic acid) (PLA), and poly(D,L-lactic-co-glycolic acid) (PLGA), have received considerable attention as possible drug delivery carriers, since the degradation products of these polymers have been found to have low toxicity. During the normal metabolic function of the body these polymers degrade into carbon dioxide and water (Mehta et al, J. Control. Rel. 29:375-384, 1994). These polymers have also exhibited excellent biocompatibility.

For prolonging the biological activity of dopamine receptor agonists and other active and delivery-enhancing agents within the inveniton, their incorporation into polymeric matrices, e.g., polyorthoesters, polyanhydrides, or polyesters, yields sustained activity and release as determined by the degradation of the polymer matrix (Heller, Formulation and Delivery of Proteins and Peptides, pp. 292-305, Cleland et al., Eds., ACS Symposium Series 567, Washington D.C., 1994; Tabata et al., Pharm. Res.10:487-496, 1993; and Cohen et al., Pharm. Res.8:713-720, 1991, each incorporated herein by reference). Although the encapsulation of biotherapeutic molecules inside synthetic polymers may stabilize them during storage and delivery, the largest obstacle of polymer-based release technology is the activity loss of the therapeutic molecules during the formulation processes that often involve heat, sonication or organic solvents (Tabata et al., Pharm. Res.10:487-496, 1993; and Jones et al., Drug Targeting and Delivery Series New Delivery Systems for Recombinant Proteins—Practical Issues from Proof of Concept to Clinic, Vol. 4, pp. 57-67, Lee et al., Eds., Harwood Academic Publishers, 1995).

Absorption-promoting polymers contemplated for use within the invention may include derivatives and chemically or physically modified versions of the foregoing types of polymers, in addition to other naturally occurring or synthetic polymers, gums, resins, and other agents, as well as blends of these materials with each other or other polymers, so long as the alterations, modifications or blending do not adversely affect the desired properties, such as water absorption, hydrogel formation, and/or chemical stability for useful application. In more detailed aspects of the invention, polymers such as nylon, acrylan and other normally hydrophobic synthetic polymers may be sufficiently modified by reaction to become water swellable and/or form stable gels in aqueous media.

Suitable polymers for use within the invention should generally be stable alone and in combination with the selected dopamine receptor agonist and optional additional biologically active agent(s) and/or delivery-enhancing agent(s), and form stable hydrogels in a range of pH conditions from about pH 1 to pH 10. More typically, they should be stable and form polymers under pH conditions ranging from about 3 to 9, without additional protective coatings. However, desired stability properties may be adapted to physiological parameters characteristic of the targeted site of delivery (e.g., nasal mucosa or secondary site of delivery such as the systemic circulation of CNS). Therefore, in certain formulations higher or lower stabilities at a particular pH and in a selected chemical or biological environment will be more desirable.

Absorption-promoting polymers of the invention may include polymers from the group of homo- and copolymers based on various combinations of the following vinyl monomers: acrylic and methacrylic acids, acrylamide, methacrylamide, hydroxyethylacrylate or methacrylate, vinylpyrrolidones, as well as polyvinylalcohol and its co- and terpolymers, polyvinylacetate, its co- and terpolymers with the above listed monomers and 2-acrylamido-2-methyl-propanesulfonic acid (AMPS®). Very useful are copolymers of the above listed monomers with copolymerizable functional monomers such as acryl or methacryl amide acrylate or methacrylate esters where the ester groups are derived from straight or branched chain alkyl, aryl having up to four aromatic rings which may contain alkyl substituents of 1 to 6 carbons; steroidal, sulfates, phosphates or cationic monomers such as N,N-dimethylaminoalkyl(meth)acrylamide, dimethylaminoalkyl(meth)acrylate, (meth)acryloxyalkyltrimethylammonium chloride, (meth)acryloxyalkyldimethylbenzyl ammonium chloride.

Additional absorption-promoting polymers for use within the invention are those classified as dextrans, dextrins, and from the class of materials classified as natural gums and resins, or from the class of natural polymers such as processed collagen, chitin, chitosan, pullalan, zooglan, alginates and modified alginates such as “Kelcoloid” (a polypropylene glycol modified alginate) gellan gums such as “Kelocogel”, Xanathan gums such as “Keltrol”, estastin, alpha hydroxy butyrate and its copolymers, hyaluronic acid and its derivatives, polylactic and glycolic acids.

A very useful class of polymers applicable within the instant invention are olefinically-unsaturated carboxylic acids containing at least one activated carbon-to-carbon olefinic double bond, and at least one carboxyl group; that is, an acid or functional group readily converted to an acid containing an olefinic double bond which readily functions in polymerization because of its presence in the monomer molecule, either in the alpha-beta position with respect to a carboxyl group, or as part of a terminal methylene grouping. Olefinically-unsaturated acids of this class include such materials as the acrylic acids typified by the acrylic acid itself, alpha-cyano acrylic acid, beta methylacrylic acid (crotonic acid), alpha-phenyl acrylic acid, beta-acryloxy propionic acid, cinnamic acid, p-chloro cinnamic acid, 1-carboxy-4-phenyl butadiene-1,3, itaconic acid, citraconic acid, mesaconic acid, glutaconic acid, aconitic acid, maleic acid, fumaric acid, and tricarboxy ethylene. As used herein, the term “carboxylic acid” includes the polycarboxylic acids and those acid anhydrides, such as maleic anhydride, wherein the anhydride group is formed by the elimination of one molecule of water from two carboxyl groups located on the same carboxylic acid molecule.

Representative acrylates useful as absorption-promoting agents within the invention include methyl acrylate, ethyl acrylate, propyl acrylate, isopropyl acrylate, butyl acrylate, isobutyl acrylate, methyl methacrylate, methyl ethacrylate, ethyl methacrylate, octyl acrylate, heptyl acrylate, octyl methacrylate, isopropyl methacrylate, 2-ethylhexyl methacrylate, nonyl acrylate, hexyl acrylate, n-hexyl methacrylate, and the like. Higher alkyl acrylic esters are decyl acrylate, isodecyl methacrylate, lauryl acrylate, stearyl acrylate, behenyl acrylate and melissyl acrylate and methacrylate versions thereof. Mixtures of two or three or more long chain acrylic esters may be successfully polymerized with one of the carboxylic monomers. Other comonomers include olefins, including alpha olefins, vinyl ethers, vinyl esters, and mixtures thereof.

Other vinylidene monomers may also be used as absorption-promoting agents within the methods and compositions of the invention, including the acrylic nitriles. Useful alpha, beta-olefinically unsaturated nitriles are preferably monoolefinically unsaturated nitriles having from 3 to 10 carbon atoms such as acrylonitrile, methacrylonitrile, and the like. Most preferred are acrylonitrile and methacrylonitrile. Acrylic amides containing from 3 to 35 carbon atoms including monoolefinically unsaturated amides also may be used. Representative amides include acrylamide, methacrylamide, N-t-butyl acrylamide, N-cyclohexyl acrylamide, higher alkyl amides, where the alkyl group on the nitrogen contains from 8 to 32 carbon atoms, acrylic amides including N-alkylol amides of alpha, beta-olefinically unsaturated carboxylic acids including those having from 4 to 10 carbon atoms such as N-methylol acrylamide, N-propanol acrylamide, N-methylol methacrylamide, N-methylol maleimide, N-methylol maleamic acid esters, N-methylol-p-vinyl benzamide, and the like.

Yet additional useful absorption promoting materials are alpha-olefins containing from 2 to 18 carbon atoms, more preferably from 2 to 8 carbon atoms; dienes containing from 4 to 10 carbon atoms; vinyl esters and allyl esters such as vinyl acetate; vinyl aromatics such as styrene, methyl styrene and chloro-styrene; vinyl and allyl ethers and ketones such as vinyl methyl ether and methyl vinyl ketone; chloroacrylates; cyanoalkyl acrylates such as alpha-cyanomethyl acrylate, and the alpha-, beta-, and gamma-cyanopropyl acrylates; alkoxyacrylates such as methoxy ethyl acrylate; haloacrylates as chloroethyl acrylate; vinyl halides and vinyl chloride, vinylidene chloride and the like; divinyls, diacrylates and other polyfunctional monomers such as divinyl ether, diethylene glycol diacrylate, ethylene glycol dimethacrylate, methylene-bis-acrylamide, allylpentaerythritol, and the like; and bis (beta-haloalkyl) alkenyl phosphonates such as bis(beta-chloroethyl) vinyl phosphonate and the like as are known to those skilled in the art. Copolymers wherein the carboxy containing monomer is a minor constituent, and the other vinylidene monomers present as major components are readily prepared in accordance with the methods disclosed herein.

When hydrogels are employed as absorption promoting agents within the invention, these may be composed of synthetic copolymers from the group of acrylic and methacrylic acids, acrylamide, methacrylamide, hydroxyethylacrylate (HEA) or methacrylate (HEMA), and vinylpyrrolidones which are water interactive and swellable. Specific illustrative examples of useful polymers, especially for the delivery of peptides or proteins, are the following types of polymers: (meth)acrylamide and 0.1 to 99 wt. % (meth)acrylic acid; (meth)acrylamides and 0.1-75 wt % (meth)acryloxyethyl trimethyammonium chloride; (meth)acrylamide and 0. 1-75 wt % (meth)acrylamide; acrylic acid and 0. 1-75 wt % alkyl(meth)acrylates; (meth)acrylamide and 0.1-75 wt % AMPS.RTM. (trademark of Lubrizol Corp.); (meth)acrylamide and 0 to 30 wt % alkyl(meth)acrylamides and 0.1-75 wt % AMPS.RTM.; (meth)acrylamide and 0.1-99 wt. % HEMA; (meth)acrylamide and 0.1 to 75 wt % HEMA and 0.1 to 99%(meth)acrylic acid; (meth)acrylic acid and 0. 1-99 wt % HEMA; 50 mole % vinyl ether and 50 mole % maleic anhydride; (meth)acrylamide and 0.1 to 75 wt % (meth)acryloxyalky dimethyl benzylammonium chloride; (meth)acrylamide and 0.1 to 99 wt % vinyl pyrrolidone; (meth)acrylamide and 50 wt % vinyl pyrrolidone and 0.1-99.9 wt % (meth)acrylic acid; (meth)acrylic acid and 0.1 to 75 wt % AMPS.RTM. and 0.1-75 wt % alkyl(meth)acrylamide. In the above examples, alkyl means C₁ to C₃₀, preferably C₁ to C₂₂, linear and branched and C₄ to C₁₆ cyclic; where (meth) is used, it means that the monomers with and without the methyl group are included. Other very useful hydrogel polymers are swellable, but insoluble versions of poly(vinyl pyrrolidone) starch, carboxymethyl cellulose and polyvinyl alcohol.

Additional polymeric hydrogel materials useful within the invention include (poly) hydroxyalkyl (meth)acrylate: anionic and cationic hydrogels: poly(electrolyte) complexes; poly(vinyl alcohols) having a low acetate residual: a swellable mixture of crosslinked agar and crosslinked carboxymethyl cellulose: a swellable composition comprising methyl cellulose mixed with a sparingly crosslinked agar; a water swellable copolymer produced by a dispersion of finely divided copolymer of maleic anhydride with styrene, ethylene, propylene, or isobutylene; a water swellable polymer of N-vinyl lactams; swellable sodium salts of carboxymethyl cellulose; and the like.

Other gelable, fluid imbibing and retaining polymers useful for forming the hydrophilic hydrogel for intranasal delivery of biologically active agents within the invention include pectin; polysaccharides such as agar, acacia, karaya, tragacenth, algins and guar and their crosslinked versions; acrylic acid polymers, copolymers and salt derivatives, polyacrylamides; water swellable indene maleic anhydride polymers; starch graft copolymers; acrylate type polymers and copolymers with water absorbability of about 2 to 400 times its original weight; diesters of polyglucan; a mixture of crosslinked poly(vinyl alcohol) and poly(N-vinyl-2-pyrrolidone); polyoxybutylene-polyethylene block copolymer gels; carob gum; polyester gels; poly urea gels; polyether gels; polyamide gels; polyimide gels; polypeptide gels; polyamino acid gels; poly cellulosic gels; crosslinked indene-maleic anhydride acrylate polymers; and polysaccharides.

Synthetic hydrogel polymers for use within the invention may be made by an infinite combination of several monomers in several ratios. The hydrogel can be crosslinked and generally possesses the ability to imbibe and absorb fluid and swell or expand to an enlarged equilibrium state. The hydrogel typically swells or expands upon delivery to the nasal mucosal surface, absorbing about 2-5, 5-10, 10-50, up to 50-100 or more times fold its weight of water. The optimum degree of swellability for a given hydrogel will be determined for different biologically active agents depending upon such factors as molecular weight, size, solubility and diffusion characteristics of the active agent carried by or entrapped or encapsulated within the polymer, and the specific spacing and cooperative chain motion associated with each individual polymer.

Hydrophilic polymers useful within the invention are water insoluble but water swellable. Such water swollen polymers as typically referred to as hydrogels or gels. Such gels may be conveniently produced from water soluble polymer by the process of crosslinking the polymers by a suitable crosslinking agent. However, stable hydrogels may also be formed from specific polymers under defined conditions of pH, temperature and/or ionic concentration, according to know methods in the art. Typically the polymers are cross-linked, that is, cross-linked to the extent that the polymers possess good hydrophilic properties, have improved physical integrity (as compared to non cross-linked polymers of the same or similar type) and exhibit improved ability to retain within the gel network both the biologically active agent of interest and additional compounds for coadministration therewith such as a cytokine or enzyme inhibitor, while retaining the ability to release the active agent(s) at the appropriate location and time.

Generally hydrogel polymers for use within the invention are crosslinked with a difunctional cross-linking in the amount of from 0.01 to 25 weight percent, based on the weight of the monomers forming the copolymer, and more preferably from 0.1 to 20 weight percent and more often from 0. 1 to 15 weight percent of the crosslinking agent. Another useful amount of a crosslinking agent is 0.1 to 10 weight percent. Tri, tetra or higher multifunctional crosslinking agents may also be employed. When such reagents are utilized, lower amounts may be required to attain equivalent crosslinking density, i.e., the degree of crosslinking, or network properties that are sufficient to contain effectively the biologically active agent(s).

The crosslinks can be covalent, ionic or hydrogen bonds with the polymer possessing the ability to swell in the presence of water containing fluids. Such crosslinkers and crosslinking reactions are known to those skilled in the art and in many cases are dependent upon the polymer system. Thus a crosslinked network may be formed by free radical copolymerization of unsaturated monomers. Polymeric hydrogels may also be formed by crosslinking preformed polymers by reacting functional groups found on the polymers such as alcohols, acids, amines with such groups as glyoxal, formaldehyde or glutaraldehyde, bis anhydrides and the like.

The polymers also may be cross-linked with any polyene, e.g. decadiene or trivinyl cyclohexane; acrylamides, such as N,N-methylene-bis (acrylamide); polyfunctional acrylates, such as trimethylol propane triacrylate; or polyfunctional vinylidene monomer containing at least 2 terminal CH.sub.2<groups, including, for example, divinyl benzene, divinyl naphthlene, allyl acrylates and the like. In certain embodiments, cross-linking monomers for use in preparing the copolymers are polyalkenyl polyethers having more than one alkenyl ether grouping per molecule, which may optionally possess alkenyl groups in which an olefinic double bond is present attached to a terminal methylene grouping (e.g., made by the etherification of a polyhydric alcohol containing at least 2 carbon atoms and at least 2 hydroxyl groups). Compounds of this class may be produced by reacting an alkenyl halide, such as allyl chloride or allyl bromide, with a strongly alkaline aqueous solution of one or more polyhydric alcohols. The product may be a complex mixture of polyethers with varying numbers of ether groups. Efficiency of the polyether cross-linking agent increases with the number of potentially polymerizable groups on the molecule. Typically, polyethers containing an average of two or more alkenyl ether groupings per molecule are used. Other cross-linking monomers include for example, diallyl esters, dimethallyl ethers, allyl or methallyl acrylates and acrylamides, tetravinyl silane, polyalkenyl methanes, diacrylates, and dimethacrylates, divinyl compounds such as divinyl benzene, polyallyl phosphate, diallyloxy compounds and phosphite esters and the like. Typical agents are allyl pentaerythritol, allyl sucrose, trimethylolpropane triacrylate, 1,6-hexanediol diacrylate, trimethylolpropane diallyl ether, pentaerythritol triacrylate, tetramethylene dimethacrylate, ethylene diacrylate, ethylene dimethacrylate, triethylene glycol dimethacrylate, and the like. Allyl pentaerythritol, trimethylolpropane diallylether and allyl sucrose provide suitable polymers. When the cross-linking agent is present, the polymeric mixtures usually contain between about 0.01 to 20 weight percent, e.g., 1%, 5%, or 10% or more by weight of cross-linking monomer based on the total of carboxylic acid monomer, plus other monomers.

In more detailed aspects of the invention, mucosal delivery of dopamine receptor agonists is enhanced by retaining the receptor agonist and, optionally, other active and/or delivery enhancing agents, in a slow-release or enzymatically or physiologically protective carrier or vehicle, for example a hydrogel that shields the active agent from the action of the degradative enzymes. In certain embodiments, the dopamine receptor agonist is bound by chemical means to the carrier or vehicle, to which may also be admixed or bound additional agents such as enzyme inhibitors, cytokines, etc. The dopamine receptor agonist may alternately be immobilized through sufficient physical entrapment within the carrier or vehicle, e.g., a polymer matrix.

Polymers such as hydrogels useful within the invention may incorporate functional linked agents such as glycosides chemically incorporated into the polymer for enhancing intranasal bioavailability of active agents formulated therewith. Examples of such glycosides are glucosides, fructosides, galactosides, arabinosides, mannosides and their alkyl substituted derivatives and natural glycosides such as arbutin, phlorizin, amygdalin, digitonin, saponin, and indican. There are several ways in which a typical glycoside may be bound to a polymer. For example, the hydrogen of the hydroxyl groups of a glycoside or other similar carbohydrate may be replaced by the alkyl group from a hydrogel polymer to form an ether. Also, the hydroxyl groups of the glycosides may be reacted to esterify the carboxyl groups of a polymeric hydrogel to form polymeric esters in situ. Another approach is to employ condensation of acetobromoglucose with cholest-5-en-3beta-ol on a copolymer of maleic acid. N-substituted polyacrylamides can be synthesized by the reaction of activated polymers with omega-aminoalkylglycosides: (1) (carbohydrate-spacer)(n)-polyacrylamide, ‘pseudopolysaccharides’; (2) (carbohydrate spacer)(n)-phosphatidylethanolamine(m)-polyacrylamide, neoglycolipids, derivatives of phosphatidylethanolamine; (3) (carbohydrate-spacer)(n)-biotin(m)-polyacrylamide. These biotinylated derivatives may attach to lectins on the nasal mucosal surface facilitate absorption of the biologically active agent, e.g., a polymer encapsulated protein or peptide.

Within more detailed aspects of the invention, dopamine receptor agonists, and, optionally, additional, secondary active agents such as protease inhibitor(s), cytokine(s), modulator(s) of intercellular junctional physiology, etc., are modified and bound to a polymeric carrier or matrix. For example, this may be accomplished by chemically binding a peptide or protein active agent and other optional agent(s) within a crosslinked polymer network. It is also possible to chemically modify the polymer separately with an interactive agent such as a glycosidal containing molecule. In certain aspects, the dopamine receptor agonist and optional secondary active agent(s), may be functionalized, i.e., wherein an appropriate reactive group is identified or is chemically added to the active agent(s). Most often an ethylenic polymerizable group is added, and the functionalized active agent is then copolymerized with monomers and a crosslinking agent using a standard polymerization method such as solution polymerization (usually in water), emulsion, suspension or dispersion polymerization. Often, the functionalizing agent is provided with a high enough concentration of functional or polymerizable groups to insure that several sites on the active agent(s) are functionalized. For example, in a polypeptide comprising 16 amine sites, it is generally desired to functionalize at least 2, 4, 5, 7, up to 8 or more of said sites.

After functionalization, the functionalized active agent(s) is/are mixed with monomers and a crosslinking agent which comprise the reagents from which the polymer of interest is formed. Polymerization is then induced in this medium to create a polymer containing the bound active agent(s). The polymer is then washed with water or other appropriate solvents and otherwise purified to remove trace unreacted impurities and, if necessary, ground or broken up by physical means such as by stirring, forcing it through a mesh, ultrasonication or other suitable means to a desired particle size. The solvent, usually water, is then removed in such a manner as to not denature or otherwise degrade the active agent(s). One desired method is lyophilization (freeze drying) but other methods are available and may be used (e.g., vacuum drying, air drying, spray drying, etc.).

To introduce polymerizable groups in peptides, proteins and other active agents within the invention, it is possible to react available amino, hydroxyl, thiol and other reactive groups with electrophiles containing unsaturated groups. For example, unsaturated monomers containing N-hydroxy succinimidyl groups, active carbonates such as p-nitrophenyl carbonate, trichlorophenyl carbonates, tresylate, oxycarbonylimidazoles, epoxide, isocyanates and aldehyde, and unsaturated carboxymethyl azides and unsaturated orthopyridyl-disulfide belong to this category of reagents. Illustrative examples of unsaturated reagents are allyl glycidyl ether, allyl chloride, allylbromide, allyl iodide, acryloyl chloride, allyl isocyanate, allylsulfonyl chloride, maleic anhydride, copolymers of maleic anhydride and allyl ether, and the like.

All of the lysine active derivatives, except aldehyde, can generally react with other amino acids such as imidazole groups of histidine and hydroxyl groups of tyrosine and the thiol groups of cystine if the local environment enhances nucleophilicity of these groups. Aldehyde containing functionalizing reagents are specific to lysine. These types of reactions with available groups from lysines, cysteines, tyrosine have been extensively documented in the literature and are known to those skilled in the art.

In the case of biologically active agents which contain amine groups, it is convenient to react such groups with an acyloyl chloride, such as acryloyl chloride, and introduce the polymerizable acrylic group onto the reacted agent. Then during preparation of the polymer, such as during the crosslinking of the copolymer of acrylamide and acrylic acid, the functionalized active agent, through the acrylic groups, is attached to the polymer and becomes bound thereto.

In additionional aspects of the invention, dopamine receptor agonists and optional additional biologically active agents and/or delivery-enhancing agents, including peptides, proteins, nucleosides, and other molecules which are bioactive in vivo, are conjugation-stabilized by covalently bonding one or more of the active or enhancing agent(s) to a polymer incorporating as an integral part thereof both a hydrophilic moiety, e.g., a linear polyalkylene glycol, and a lipophilic moiety (see, e.g., U.S. Pat. No. 5,681,811, incorporated herein by reference). In one aspect, a biologically active agent is covalently coupled with a polymer comprising (i) a linear polyalkylene glycol moiety and (ii) a lipophilic moiety, wherein the active agent, linear polyalkylene glycol moiety, and the lipophilic moiety are conformationally arranged in relation to one another such that the active therapeutic agent has an enhanced in vivo resistance to enzymatic degradation (i.e., relative to its stability under similar conditions in an unconjugated form devoid of the polymer coupled thereto). In another aspect, the conjugation-stabilized formulation has a three-dimensional conformation comprising the biologically active agent covalently coupled with a polysorbate complex comprising (i) a linear polyalkylene glycol moiety and (ii) a lipophilic moiety, wherein the active agent, the linear polyalkylene glycol moiety and the lipophilic moiety are conformationally arranged in relation to one another such that (a) the lipophilic moiety is exteriorly available in the three-dimensional conformation, and (b) the active agent in the composition has an enhanced in vivo resistance to enzymatic degradation.

In a further related aspect, a multiligand conjugated complex is provided which comprises a dopamine receptor agonist and/or other biologically active or delivery-enhancing agent covalently coupled with a triglyceride backbone moiety through a polyalkylene glycol spacer group bonded at a carbon atom of the triglyceride backbone moiety, and at least one fatty acid moiety covalently attached either directly to a carbon atom of the triglyceride backbone moiety or covalently joined through a polyalkylene glycol spacer moiety (see, e.g., U.S. Pat. No. 5,681,811, incorporated herein by reference). In such multiligand conjugated therapeutic agent complex, the alpha′ and beta carbon atoms of the triglyceride bioactive moiety may have fatty acid moieties attached by covalently bonding either directly thereto, or indirectly covalently bonded thereto through polyalkylene glycol spacer moieties. Alternatively, a fatty acid moiety may be covalently attached either directly or through a polyalkylene glycol spacer moiety to the alpha and alpha′ carbons of the triglyceride backbone moiety, with the bioactive therapeutic agent being covalently coupled with the gamma-carbon of the triglyceride backbone moiety, either being directly covalently bonded thereto or indirectly bonded thereto through a polyalkylene spacer moiety. It will be recognized that a wide variety of structural, compositional, and conformational forms are possible for the multiligand conjugated therapeutic agent complex comprising the triglyceride backbone moiety, within the scope of the invention. It is further noted that in such a multiligand conjugated therapeutic agent complex, the biologically active agent(s) may advantageously be covalently coupled with the triglyceride modified backbone moiety through alkyl spacer groups, or alternatively other acceptable spacer groups, within the scope of the invention. As used in such context, acceptability of the spacer group refers to steric, compositional, and end use application specific acceptability characteristics.

In yet additional aspects of the invention, a conjugation-stabilized complex is provided which comprises a polysorbate complex comprising a polysorbate moiety including a triglyceride backbone having covalently coupled to alpha, alpha′ and beta carbon atoms thereof functionalizing groups including (i) a fatty acid group; and (ii) a polyethylene glycol group having a biologically active agent or moiety covalently bonded thereto, e.g., bonded to an appropriate functionality of the polyethylene glycol group (see, e.g., U.S. Pat. No. 5,681,811, incorporated herein by reference). Such covalent bonding may be either direct, e.g., to a hydroxy terminal functionality of the polyethylene glycol group, or alternatively, the covalent bonding may be indirect, e.g., by reactively capping the hydroxy terminus of the polyethylene glycol group with a terminal carboxy functionality spacer group, so that the resulting capped polyethylene glycol group has a terminal carboxy functionality to which the dopamine receptor agonist or other biologically active or delivery-enhancing agent or moiety may be covalently bonded.

In yet additional aspects of the invention, a stable, aqueously soluble, conjugation-stabilized complex is provided which comprises a dopamine receptor agonist and/or other biologically active or delivery-enhancing agent covalently coupled to a physiologically compatible polyethylene glycol (PEG) modified glycolipid moiety. In such complex, the biologically active agent may be covalently coupled to the physiologically compatible PEG modified glycolipid moiety by a labile covalent bond at a free amino acid group of the active agent, wherein the labile covalent bond is scissionable in vivo by biochemical hydrolysis and/or proteolysis. The physiologically compatible PEG modified glycolipid moiety may advantageously comprise a polysorbate polymer, e.g., a polysorbate polymer comprising fatty acid ester groups selected from the group consisting of monopalmitate, dipalmitate, monolaurate, dilaurate, trilaurate, monoleate, dioleate, trioleate, monostearate, distearate, and tristearate. In such complex, the physiologically compatible PEG modified glycolipid moiety may suitably comprise a polymer selected from the group consisting of polyethylene glycol ethers of fatty acids, and polyethylene glycol esters of fatty acids, wherein the fatty acids for example comprise a fatty acid selected from the group consisting of lauric, palmitic, oleic, and stearic acids.

In other detailed aspects of the invention, mucosal delivery of dopamine receptor agonists is enhanced by combining or coordinately administering the dopamine receptor agonist with a polypropylene-based or other membrane penetration-enhancing polymer or copolymer (e.g., a polypropylene glycol-(PPG)-PEG copolymer). A variety of such polymers (e.g., polypropylene oxides, polypropylene glycols) are known in the art and can provide for enhanced membrane permeation of dopamine receptor agonists (see, e.g., Vandorpe et al., Biomaterials 18:1147-1152, 1997; Kajihara et al., Biosci. Biotechnol. Biochem 61:197-9, 1997; Yeh et al., Pharm. Res. 13:1693-8, 1996; Rogers et al., J. Chromatogr. B. Biomed. Appl. 680:231-6, 1996; Kronick, Pharmacol. Res. Commun. 10:257-9, 1978, each incorporated herein by reference.

Bioadhesive Delivery Vehicles and Methods

In certain aspects of the invention, the methods and compositions for mucosal delivery of dop amine receptor agonists herein incorporate an effective amount of a nontoxic bioadhesive as a coordinately administered adjunct compound or carrier to enhance mucosal delivery of a dop amine receptor agonist. Alternatively, safe and effective bioadhesive agents may be incorporated as processing agents within the formulation methods of the invention, or as additives within the formulations of the invention to provide improved formulations for mucosal delivery of dopamine receptor agonists.

Bioadhesive agents in this context exhibit general or specific adhesion to one or more components or surfaces of mucosal epithelia. The bioadhesive maintains a desired concentration gradient of the dop amine receptor agonist across the mucosa to ensure penetration into or through the musosal epithelium. Typically, employment of a bio adhesive within the methods and compositions of the invention yields a two- to five-fold, often a five- to ten-fold increase in permeability for dopamine receptor agonists into or through mucosal epithelia. This enhancement of epithelial permeation often permits effective transmucosal delivery of dopamine receptor agonists, as well as optionalm, additional biologically active agents including large macromolecules, for example to the basal portion of the nasal epithelium or into the adjacent extracellular compartments, the systemic circulation or central nervous system.

This enhanced delivery provides for greatly improved effectiveness of delivery of dopamine receptor agonists and other, optional bioactive peptides, proteins and other macromolecular therapeutic species. These results will depend in part on the hydrophilicity of the dopamine receptor agonist or other compound, whereby greater penetration will be achieved with hydrophilic species compared to water insoluble compounds. In addition to these effects, employment of bioadhesives to enhance drug persistence at the mucosal surface can elicit a reservoir mechanism for protracted drug delivery, whereby compounds not only penetrate across the mucosal tissue but also back-diffuse toward the mucosal surface once the material at the surface is depleted.

A variety of suitable bioadhesives are disclosed in art for mucosal administration (see, e.g., U.S. Pat. Nos. 3,972,995; 4,259,314; 4,680,323; 4,740,365; 4,573,996; 4,292,299; 4,715,369; 4,876,092; 4,855,142; 4,250,163; 4,226,848; 4,948,580; U.S. Pat. Reissue No. 33,093; and Robinson, 18 Proc. Intern. Symp. Control. Rel. Bioact. Mater. 75 (1991), each incorporated herein by reference), which find use within the novel methods and compositions of the invention. The potential of various bioadhesive polymers as a mucosal delivery platform within the methods and compositions of the invention can be readily assessed by determining their ability to retain and release a specific biologically active agent, e.g., a therapeutic peptide or protein, as well as by their capacity to interact with the nasal mucosal surfaces following incorporation of the active agent therein. In addition, well known methods will be applied to determine the biocompatibility of selected polymers with the tissue at the site of mucosal administration. One aspect of polymer biocompatibility is the potential effect for the polymer to induce a cytokine response. In certain circumstances, implanted polymers have been shown to induce the release of inflammatory cytokines from adhering cells, such as monocytes and macrophages. Similar potential adverse reactions of mucosal epithelial cells in contact with candidate bioadhesive polymers will be determined using routine in vitro and in vivo assays. Since epithelial cells have the ability to secrete a number of cytokines, the induction of cytokine responses in epithelial cells will often provide an adequate measure of biocompatibility of a selected polymer delivery platform.

When the mucosal site of administration is covered by mucus (i.e., in the absence of mucolytic or mucus-clearing treatment), it can serve as a connecting link to underlying mucosal epithelium. Therefore, the term “bioadhesive” as used herein also covers mucoadhesive compounds useful for enhancing intranasal delivery of biologically active agents within the invention. However, adhesive contact to mucosal tissue which is mediated through adhesion to a mucus gel layer may be limited by incomplete or transient attachment between the mucus layer and the underlying tissue, particularly at nasal surfaces where rapid mucus clearance occurs. In this regard, mucin glycoproteins are continuously secreted and, immediately after their release from cells or glands, form a viscoelastic gel. The luminal surface of the adherent gel layer, however, is continuously eroded by mechanical, enzymatic and/or ciliary action. Where such activities are more prominent, or where longer adhesion times are desired, the coordinate administration methods and combinatorial formulation methods of the invention may further incorporate mucolytic and/or ciliostatic methods or agents as disclosed herein.

Bioadhesion involves the attachment of a natural or synthetic polymer to a biological substrate. It serves within the methods and compositions of the invention as a practical method for drug immobilization or localization at the nasal mucosal surface, thereby providing for enhanced absorption and better controlled drug delivery. In the latter context, the use of bioadhesive polymers and other combinatorial formulations within the invention provides for maintenance of a relatively constant effective drug concentration at the target site for action for an extended time period. For optimal performance, drug concentrations at the target site (e.g., a selected tissue or compartment such as the brain or systemic circulation) should be maintained above the effective concentration level for the drug and below a toxic or otherwise excessive dosage level. Using conventional formulations, when a drug is administered to a patient, particularly intravenously, the initial concentration of the drug in the body will peak above a toxic level before gradually diminishing to an ineffective level due to degradation, excretion and other factors.

Bioadhesive and other delivery components within the methods and compositions of the invention can improve the effectiveness of a treatment by helping maintain the drug concentration between effective and toxic levels, by inhibiting dilution of the drug away from the delivery point, and improving targeting and localization of the drug. In this context, bioadhesion increases the intimacy and duration of contact between a drug-containing polymer and the nasal mucosal surface. The combined effects of this enhanced, direct drug absorption, and the decrease in excretion rate that results from reduced diffusion and improved localization, significantly enhances bioavailability of the drug and allows for a smaller dosage and less frequent administration.

Typically, mucoadhesive polymers for use within the invention are natural or synthetic macromolecules which adhere to wet mucosal tissue surfaces by complex, but non-specific, mechanisms. In addition to these mucoadhesive polymers, the invention also provides methods and compositions incorporating bioadhesives that adhere directly to a cell surface, rather than to mucus, by means of specific, including receptor-mediated, interactions. One example of bioadhesives that function in this specific manner is the group of compounds known as lectins. These are glycoproteins with an ability to specifically recognize and bind to sugar molecules, e.g. glycoproteins or glycolipids, which form part of intranasal epithelial cell membranes and can be considered as “lectin receptors”.

In various embodiments, the coordinate administration methods of the instant invention optionally incorporate bioadhesive materials that yield prolonged residence time at the nasal mucosal surface or target site of action of the biologically active agent. Alternatively, the bioadhesive material may otherwise facilitate intranasal absorption of the biologically active agent, e.g., by facilitating localization of the active agent to a selected target site of activity (e.g., bloodstream or CNS). In additional aspects, adjunct delivery or combinatorial formulation of bioadhesive agent within the methods and compositions of the invention intensify contact of the dopamine receptor agonist or other biologically active agent with the mucosa, including by increasing epithelial permeability, (e.g., to effectively increase the drug concentration gradient). In further alternate embodiments, bioadhesives and other polymers disclosed herein serve to inhibit proteolytic or other enzymes that might degrade the biologically active agent. For a review of different approaches to bioadhesion that are useful within the coordinate administration, multi-processing and/or combinatorial formulation methods and compositions of the invention, see, e.g., Lehr C. M., Eur J. Drug Metab. Pharmacokinetics 21(2:139-148, 1996 (incorporated herein by reference).

In certain aspects of the invention, bioadhesive materials for enhancing mucosal delivery of dopamine receptor agonists and other biologically active agents comprise a matrix of a hydrophilic, e.g., water soluble or swellable, polymer or a mixture of polymers that can adhere to a wet mucous surface. These adhesives may be formulated as ointments, hydrogels (see above) thin films, and other application forms. Often, these adhesives have the biologically active agent mixed therewith to effectuate slow release or local delivery of the active agent. Some are formulated with additional ingredients to facilitate penetration of the active agent through the mucosa, e.g., into the circulatory system or central nervous system of the individual.

Various polymers, both natural and synthetic ones, show significant binding to mucus and/or mucosal epithelial surfaces under physiological conditions. The strength of this interaction can readily be measured by mechanical peel or shear tests. A variety of suitable test methods and instruments to serve such purposes are known in the art (see, e.g., Gu et al., Crit. Rev. Ther. Drug Carrier Syst. 5:21-67, 1988; Duchene et al., Drug Dev. Ind. Pharm. 14:283-318, 1988, incorporated herein by reference). When applied to a humid mucosal surface, many dry materials will spontaneously adhere, at least slightly. After such an initial contact, some hydrophilic materials start to attract water by adsorption, swelling or capillary forces, and if this water is absorbed from the underlying substrate or from the polymer-tissue interface, the adhesion may be sufficient to achieve the goal of enhancing mucosal absorption of dopamine receptor agonists and other, optional biologically active agents (see, e.g., Al-Dujaili et al., Int. J. Pharm. 34:75-79, 1986; Marvola et al., J. Pharm. Sci. 72:1034-1036, 1983; Marvola et al., J. Pharm. Sci. 71:975-977, 1982; and Swisher et al., Int. J. Pharm. 22:219, 1984; Chen, et al., Adhesion in Biological Systems, p. 172, Manly, Ed., Academic Press, London, 1970, each incorporated herein by reference). Such ‘adhesion by hydration’ can be quite strong, but formulations adapted to employ this mechanism must account for swelling which continues as the dosage transforms into a hydrated mucilage. This is projected for many hydrocolloids useful within the invention, especially some cellulose-derivatives, which are generally non-adhesive when applied in pre-hydrated state. Nevertheless, bioadhesive drug delivery systems for mucosal administration are effective within the invention when such materials are applied in the form of a dry polymeric powder, microsphere, or film-type delivery form.

Other polymers adhere to mucosal surfaces not only when applied in dry, but also in fully hydrated state, and in the presence of excess amounts of water. The selection of a mucoadhesive thus requires due consideration of the conditions, physiological as well as physico-chemical, under which the contact to the tissue will be formed and maintained. In particular, the amount of water or humidity usually present at the intended site of adhesion, and the prevailing pH, are known to largely affect the mucoadhesive binding strength of different polymers.

Several polymeric bioadhesive drug delivery systems have been fabricated and studied in the past 20 years, not always with success. A variety of such carriers are, however, currently used in clinical applications involving dental, orthopedic, ophthalmological, and surgical uses. For example, acrylic-based hydrogels have been used extensively for bioadhesive devices. Acrylic-based hydrogels are well-suited for bioadhesion due to their flexibility and nonabrasive characteristics in the partially swollen state which reduce damage-causing attrition to the tissues in contact (Park et al., J. Control. Release 2:47-57, 1985, incorporated herein by reference). Furthermore, their high permeability in the swollen state allows unreacted monomer, un-crosslinked polymer chains, and the initiator to be washed out of the matrix after polymerization, which is an important feature for selection of bioadhesive materials for use within the invention. Acrylic-based polymer devices exhibit very high adhesive bond strength, as determined by various known methods (Park et al., J. Control. Release 2:47-57, 1985; Park et al., Pharm. Res. 4:457-464, 1987; and Chung et al., J. Pharm. Sci. 74:399-405, 1985, each incorporated herein by reference).

For controlled mucosal delivery of dopamine receptor agonists and other, optional biologically active agents, including peptide and protein drugs, the methods and compositions of the invention optionally include the use of carriers, e.g., polymeric delivery vehicles, that function in part to shield the dopamine receptor agonist or other biologically active agent from enzymatic breakdown, while at the same time providing for enhanced penetration of the active agent(s) into or through the mucosa. In this context, bioadhesive polymers have demonstrated considerable potential for enhancing oral drug delivery. As an example, the bioavailability of 9-desglycinamide, 8-arginine vasopressin (DGAVP) intraduodenally administered to rats together with a 1% (w/v) saline dispersion of the mucoadhesive poly(acrylic acid) derivative polycarbophil, was 3-5-fold increased compared to an aqueous solution of the peptide drug without this polymer (Lehr et al., J. Pharm. Pharmacol.44:402-407, 1992, incorporated herein by reference). In this study, the drug was not bound to or otherwise integrally associated with the mucoadhesive polymer in the formulation, which would therefore not be expected to yield enhanced peptide absorption via prolonged residence time or intensified contact to the mucosal surface. Thus, certain bioadhesive polymers for use within the invention will directly enhance the permeability of the epithelial absorption barrier in part by protecting the dopamine receptor agonist and/or other active agent, e.g., peptide or protein, from enzymatic degradation.

Recent studies have shown that mucoadhesive polymers of the poly(acrylic acid)-type are potent inhibitors of some intestinal proteases (Lueβen et al., Pharm. Res. 12:1293-1298, 1995; Lueβen et al., J. Control. Rel. 29:329-338, 1994; and Bai et al., J. Pharm. Sci. 84:1291-1294; 1995, incorporated herein by reference). The mechanism of enzyme inhibition is explained by the strong affinity of this class of polymers for divalent cations, such as calcium or zinc, which are essential cofactors of metallo-proteinases, such as trypsin and chymotrypsin. Depriving the proteases of their cofactors by poly(acrylic acid) was reported to induce irreversible structural changes of the enzyme proteins which were accompanied by a loss of enzyme activity. At the same time, other mucoadhesive polymers (e.g., some cellulose derivatives and chitosan) may not inhibit proteolytic enzymes under certain conditions. In contrast to other enzyme inhibitors contemplated for use within the invention (e.g. aprotinin, bestatin), which are relatively small molecules, the trans-nasal absorption of inhibitory polymers is likely to be minimal in light of the size of these molecules, and thereby eliminate possible adverse side effects. Thus, mucoadhesive polymers, particularly of the poly(acrylic acid)-type, may serve both as an absorption-promoting adhesive and enzyme-protective agent to enhance controlled delivery of dopamine receptor agonists as well as peptide and protein drugs, especially when safety concerns are considered.

In addition to protecting against enzymatic degradation, bioadhesives and other polymeric or non-polymeric absorption-promoting agents for use within the invention may directly increase mucosal permeability to biologically active agents. To facilitate the transport of dopamine receptor agonists, as well as large and hydrophilic molecules, such as peptides and proteins, across the mucosal epithelial barrier, mucoadhesive polymers and other agents have been postulated to yield enhanced permeation effects beyond what is accounted for by prolonged premucosal residence time of the delivery system. For example, nasal administration of insulin to non-primate mammals in the presence of mucoadhesive starch microspheres yielded a steeply enhanced early absorption peak, followed by a continuous decline (Bjork et al., Int. J. Pharm. 47:233-238, 1988; Farraj et al., J. Control. Rel. 13:253-262, 1990, each incorporated herein by reference). The time course of drug plasma concentrations reportedly suggested that the bioadhesive microspheres caused an acute, but transient increase of insulin permeability across the nasal mucosa. In other studies using in vitro cultured epithelial cell monolayers (Bjork et al., J. Drug Targeting, 1995, incorporated herein by reference), it was reported that dry, swellable materials such as starch microspheres induce reversible focal dilations of the tight junctions, allowing for enhanced drug transport along the paracellular route. According to this adhesion-dehydration theory, the hydrophilic polymer, applied as a dry powder, absorbs water from the mucosal tissue in such a way that the epithelial cells are dehydrated and shrink until the normally tight intercellular junctions between the cells become physically separated. Because this effect is of relatively short duration and appears to be completely reversible, it provides yet another useful tool for incorporation within the coordinate administration, processing and/or combinatorial or coordinate formulations and methods of the invention.

Other mucoadhesive polymers for use within the invention, for example chitosan, reportedly enhance the permeability of certain mucosal epithelia even when they are applied as an aqueous solution or gel (Lehr et al., Int. J. Pharmaceut. 78:43-48, 1992; Illum et al., Pharm. Res.11:1186-1189, 1994; Artursson et al., Pharm. Res. 11:1358-1361, 1994; and Borchard, et al., J. Control. Release 39:131-138, 1996, each incorporated herein by reference). In one study, absorption of the peptide drugs insulin and calcitonin, and the hydrophilic compound phenol red, from an aqueous gel base of poly(acrylic acid) was reported after rectal, vaginal and nasal administration (Morimoto et al., Int. J. Pharm. 14:149-157, 1983; and Morimoto et al., J. Pharmacobiodyn. 10:85-91, 1987, each incorporated herein by reference). Another mucoadhesive polymer reported to directly affect epithelial permeability is hyaluronic acid. In particular, hyaluronic acid gel formulation reportedly enhanced nasal absorption of vasopressin and some of its analogues (Morimoto et al., Pharm. Res.8:471-474, 1991, incorporated herein by reference). Hyaluronic acid was also reported to increase the absorption of insulin from the conjunctiva in diabetic dogs (Nomura, et al., J. Pharm. Pharmacol. 46:768-770, 1994). Ester derivatives of hyaluronic acid in the form of lyophilized microspheres were described as a nasal delivery system for insulin (Illum et al., J. Contr. Rel. 29:133-141, 1994).

A particularly useful bioadhesive agent within the coordinate administration, multi-processing and/or combinatorial formulation methods and compositions of the invention is chitosan, as well as its analogs and derivatives. Chitosan is a non-toxic, biocompatible and biodegradable polymer that is widely used for pharmaceutical and medical applications because of its favorable properties of low toxicity and good biocompatibility (Yomota, Pharm. Tech. Japan 10:557-564, 1994, incorporated herein by reference). It is a natural polyaminosaccharide prepared from chitin by N-deacetylation with alkali. A wide variety of biomedical uses for chitosan have been reported over the last two decades, based for example on its reported wound healing, antimicrobial and hemostatic properties (Kas, J. Microencapsulation 14:689-711, 1997, incorporated herein by reference). Chitosan has also been used as a pharmaceutical excipient in conventional dosage forms as well as in novel applications involving bioadhesion and transmucosal drug transport (Illum, Pharm. Res. 15:1326-1331, 1998; and Olsen et al., Chitin and Chitosan-sources, Chemistry Biochemistry, Physical Properties and Applications, pp. 813-828, Skjak-Braek et al., Eds., Elsevier, London, 1989, each incorporated herein by reference). Furthermore, chitosan has been reported to promote absorption of small polar molecules and peptide and protein drugs through nasal mucosa in animal models and human volunteers (Illum et al., Pharm. Res. 11:1186-1189, 1994, incorporated herein by reference). Other studies have shown an enhancing effect on penetration of compounds across the intestinal mucosa and cultured Caco-2 cells (Schipper et al., Pharm. res. 14:23-29, 1997; and Kotze et al., Int. J. Pharm. 159:243-253, 1997, each incorporated herein by reference). Chitosan has also been proposed as a bioadhesive polymer for use in oral mucosal drug delivery (Miyazaki et al., Biol. Pharm. Bull. 17:745-747, 1994; Ikinci et al., Advances in Chitin Science, Vol. 4, Peter et al., Eds., University of Potsdam, in press; Senel, et al., Int. J. Pharm. 193:197-203, 2000; Needleman, et al., J. Clin. Periodontol.24:394-400, 1997, each incorporated herein by reference). Initial studies showed that chitosan has an extended retention time on the oral mucosa (Needleman et al., J. Clin. Periodontol. 25:74-82, 1998) and with its antimicrobial properties and biocompatibility is an excellent candidate for the treatment of oral mucositis. More recently, Senel et al., Biomaterials 21:2067-2071, 2000 (incorporated herein by reference) reported that chitosan provides an effective gel carrier for delivery of the bioactive peptide, transforming growth factor-β (TGF-β).

As used within the methods and compositions of the invention, chitosan increases the retention of dopamine receptor agonists and other, optional biologically active agents at a mucosal site of application. This is thought to be mediated in part by a positive charge characteristic of chitosan, which may influence epithelial permeability even after physical removal of chitosan from the surface (Schipper et al., Pharm. Res. 14:23-29, 1997, incorporated herein by reference). Another mechanism of action of chitosan for improving transport of biologically active agents across mucosal membranes may be attributed to transient opening of the tight junctions in the cell membrane to allow polar compounds to penetrate (Illum et al., Pharm. Res.11:1186-1189, 1994; Lueben et al., J. Control. Rel. 29:329-338, 1994, each incorporated herein by reference). Chitosan may also increase the thermodynamic activity of other absorption-promoting agents used in certain formulations of the invention, resulting in enhanced penetration. Lastly, as chitosan has been reported to disrupt lipid micelles in the intestine (Muzzarelli et al., EUCHIS'99, Third International Conference of the European Chitin Society, Abstract Book, ORAD-PS-059, Potsdam, Germany, 1999), its absorption-promoting effects may be due in part to its interference with the lipid organization in the mucosal epithelium.

As with other bioadhesive gels provided herein, the use of chitosan can reduce the frequency of application and the amount of dopamine receptor agonists and other, optional biologically active agents administered while yielding an effective delivery amount or dose. This mode of administration can also improve patient compliance and acceptance. The occlusion and lubrication of chitosan and other bioadhesive gels is expected to reduce the discomfort of inflammatory, allergic and ulcerative conditions of the nasal mucosa. In addition, chitosan acts non-specifically on certain deleterious microorganisms, including fungi (Knapczyk, Chitin World, pp. 504-511, Karnicki et al., Eds., Wirtschaftverlag N W, Germany, 1994, incorporated herein by reference), and may also beneficially stimulate cell proliferation and tissue organization by acting as an inductive primer to repair and physiologically rebuild damaged tissue (Muzzarelli et al. (Biomaterials 10:598-603, 1989, incorporated herein by reference).

As further provided herein, the methods and compositions of the invention will optionally include a novel chitosan derivative or chemically modified form of chitosan. One such novel derivative for use within the invention is denoted as a β-[1→4]-2-guanidino-2-deoxy-D-glucose polymer (poly-GuD). Chitosan is the N-deacetylated product of chitin, a naturally occurring polymer that has been used extensively to prepare microspheres for oral and intra-nasal formulations. The chitosan polymer has also been proposed as a soluble carrier for parenteral drug delivery. Within one aspect of the invention, o-methylisourea is used to convert a chitosan amine to its ganidinium moiety. The gaunidinium compound is prepared, for example, by the reaction between equi-normal solutions of chitosan and o-methylisourea at pH above 8.0, as depicted by the equation shown in FIG. 1.

The guanidinium product is -[14]-guanidino-2-deoxy-D-glucose polymer. It is abbreviated as Poly-GuD in this context (Monomer F. W. of Amine in Chitosan =161; Monomer F. W. of Guanidinium in Poly-GuD =203).

One exemplary Poly-GuD preparation method for use within the inveniton involves the following protocol.

Solutions:

Preparation of 0.5% Acetic Acid Solution (0.088N):

Pipette 2.5 mL glacial acetic acid into a 500 mL volumetric flask, dilute to volume with purified water.

Preparation of 2N NaOH Solution:

Transfer about 20 g NaOH pellets into a beaker with about 150 mL of purified water. Dissolve and cool to room temperature. Transfer the solution into a 250-mL volumetric flask, dilute to volume with purified water.

Preparation of O-Methylisourea Sulfate (0.4N Urea Group Equivalent):

Transfer about 493 mg of O-methylisourea sulfate into a 10-mL volumetric flask, dissolve and dilute to volume with purified water.

The pH of the solution is 4.2

Preparation of Barium Chloride Solution (0.2M):

Transfer about 2.086 g of Barium chloride into a 50-mL volumetric flask, dissolve and dilute to volume with purified water.

Preparation of Chitosan Solution (0.06N Amine Equivalent):

Transfer about 100 mg Chitosan into a 50 mL beaker, add 10 mL 0.5% Acetic Acid (0.088 N). Stir to dissolve completely.

The pH of the solution is about 4.5

Preparation of O-Methylisourea Chloride Solution (0.2N Urea Group Equivalent):

Pipette 5.0 mL of O-methylisourea sulfate solution (0.4 N urea group equivalent) and 5 mL of 0.2M Barium chloride solution into a beaker. A precipitate is formed. Continue to mix the solution for additional 5 minutes. Filter the solution through 0.45 m filter and discard the precipitate. The concentration of O-methylisourea chloride in the supernatant solution is 0.2 N urea group equivalent.

The pH of the solution is 4.2.

Procedure:

Add 1.5 mL of 2 N NaOH to 10 mL of the chitosan solution (0.06N amine equivalent) prepared as described in Section 2.5. Adjust the pH of the solution with 2N NaOH to about 8.2 to 8.4. Stir the solution for additional 10 minutes. Add 3.0 mL O-methylisourea chloride solution (0.2N urea group equivalent) prepared as described above. Stir the solution overnight.

Adjust the pH of solution to 5.5 with 0.5% Acetic Acid (0.088N).

Dilute the solution to a final volume of 25 mL using purified water.

The Poly-GuD concentration in the solution is 5 mg/mL, equivalent to 0.025 N (guanidium group).

Additional compounds classified as bioadhesive agents for use within the present invention act by mediating specific interactions, typically classified as “receptor-ligand interactions” between complementary structures of the bioadhesive compound and a component of the mucosal epithelial surface. Many natural examples illustrate this form of specific binding bioadhesion, as exemplified by lectin-sugar interactions. Lectins are (glyco)proteins of non-immune origin which bind to polysaccharides or glycoconjugates. By virtue of this binding potential, lectins may bind or agglutinate cells (Goldstein et al., Nature 285:66, 1980). Lectins are commonly of plant or bacterial origin, but are also produced by higher animals (so-called ‘endogenous or ‘reverse’ lectins), including mammals (Sharon et al., Lectins, Chapman and Hall, London, 1989; and Pasztai et al., Lectins. Biomedical Perspectives, Taylor & Francis, London, 1995, incorporated herein by reference).

Several plant lectins have been investigated as possible pharmaceutical absorption-promoting agents. One plant lectin, Phaseolus vulgaris hemagglutinin (PHA), exhibits high oral bioavailability of more than 10% after feeding to rats (Pusztai et al., Biochem. Soc. Trans. 17:81-82, 1988, incorporated herein by reference). However, PHA has been reported to cause digestive disorders following oral administration, and these side effects must be determined to be minimized by any nasal therapeutic application herein. In contrast, tomato (Lycopersicon esculeutum) lectin (TL) appears safe for various modes of administration. This glycoprotein (approximately 70 kDa) resists digestion and binds to rat intestinal villi without inducing any deleterious effects (Kilpatrick, et al., FEBS Lett. 185:5-10, 1985; Woodley et al., Int. J. Pharm. 110: 127-136, 1994; and Int. J. Pharm. 107:223-230, 1994, each incorporated herein by reference). However, GI transit of this radiolabeled lectin after intragastric administration to rats was not delayed compared to controls, and other studies showed that TL has a strong cross-reactivity with gastrointestinal mucus glycoproteins (Lehr, et al., Pharm. Res. 9:547-553, 1992). Thus, in spite of its favorable safety profile, the use of TL as a gastrointestinal bioadhesive, even though its action is “specific” (i.e., receptor-mediated) is limited by non-specific interactions with mucus—promoting rapid clearance.

Therefore, the invention provides for coordinate administration or combinatorial formulation of non-toxic lectins identified or obtained by modification of existing lectins which have a high specific affinity for nasal epithelial cells, but low cross reactivity with nasal mucus. In this regard, detailed teachings regarding lectin structure-activity relationships will allow selection of non-toxic, strongly bioadhesive candidates to produce optimized lectins for therapeutic purposes, which undertaking will be further facilitated by methods of recombinant gene technology (see, e.g., Lehr et al., Lectins: Biomedical Perspectives, pp. 117-140, Pustai et al., Eds., Taylor and Francis, London, 1995, incorporated herein by reference). In additional embodiments of the invention, mucolytic agents and/or ciliostatic agents are coordinately administered or combinatorially formulated with a biologically active agent and a lectin or other specific binding bioadhesive—in order to counter the effects of non-specific binding of the bioadhesive to mucus.

In addition to the use of lectins, certain antibodies or amino acid sequences exhibit high affinity binding to complementary elements on mucosal cell surfaces. Thus, for example, various adhesive amino acids sequences such as Arg-Gly-Asp and others, if attached to a carrier matrix, will promote adhesion by binding with specific cell surface glycoproteins. In other embodiments, adhesive ligand components are integrated in a carrier or delivery vehicle which selectively adhere to a particular cell type, or diseased target tissue. For example, certain diseases cause changes in cell surface glycoproteins. These distinct structural alterations can be readily targeted by complementary amino acid sequences bound to a drug delivery vehicle within the invention. In exemplary aspects, well known cancer-specific markers (e.g., CEA, HER2) may be targeted by complementary antibodies or peptides for specific drug targeting to diseased cells.

The foregoing bioadhesive agents are useful in the coordinate administration methods of the instant invention, which optionally incorporate an effective amount and form of a bioadhesive agent to prolong persistence or otherwise increase mucosal absorption of dopamine receptor agonists and other, optional biologically active agents. The bioadhesive agents may be coordinately administered as adjunct compounds or as additives within the combinatorial formulations of the invention. In certain embodiments, the bioadhesive agent acts as a ‘pharmaceutical glue’, whereas in other embodiments adjunct delivery or combinatorial formulation of the bioadhesive agent serves to intensify contact of the dopamine receptor agonist or other biologically active agent with the mucosa, in some cases by promoting specific receptor-ligand interactions with epithelial cell “receptors”, and in others by increasing epithelial permeability to significantly increase the drug concentration gradient measured at a target site of delivery (e.g., the CNS or in the systemic circulation). Yet additional bioadhesive agents for use within the invention act as enzyme (e.g., protease) inhibitors to enhance the stability of intranasally administered biotherapeutic agents delivered coordinately or in a combinatorial formulation with the bioadhesive agent.

Liposomes and Micellar Delivery Vehicles

The coordinate administration methods and combinatorial formulations of the instant invention optionally incorporate effective lipid or fatty acid based carriers, processing agents, or delivery vehicles, to provide improved formulations for mucosal delivery of dopamine receptor agonists and, optionally, other biotherapeutic compounds. For example, a variety of formulations and methods are provided for mucosal delivery which comprise a dopamine receptor agonist and, optionally, one or more additional biologically active agent(s), such as a peptide or protein, admixed or encapsulated by, or coordinately administered with, a liposome, mixed micellar carrier, or emulsion, to enhance chemical and physical stability and increase the half life of the dopamine receptor agonist or other biologically active agent(s) (e.g., by reducing susceptibility to proteolysis, chemical modification and/or denaturation) upon mucosal delivery.

Within certain aspects of the invention, specialized delivery systems for dopamine receptor agonists and other, optional biologically active agents comprise small lipid vesicles known as liposomes (see, e.g., Chonn et al., Curr. Opin. Biotechnol. 6:698-708, 1995; Lasic, Trends Biotechnol. 16:307-321, 1998; and Gregoriadis, Trends Biotechnol. 13:527-537, 1995, each incorporated herein by reference). These are typically made from natural, biodegradable, non-toxic, and non-immunogenic lipid molecules, and can efficiently entrap or bind drug molecules, including peptides and proteins, into, or onto, their membranes. The attractiveness of liposomes as a peptide and protein delivery system within the invention is increased by the fact that the encapsulated proteins can remain in their preferred aqueous environment within the vesicles, while the liposomal membrane protects them against proteolysis and other destabilizing factors. Even though not all liposome preparation methods known are feasible in the encapsulation of peptides and proteins due to their unique physical and chemical properties, several methods allow the encapsulation of these macromolecules without substantial deactivation (see, e.g., Weiner, Immunomethods 4:201-209, 1994, incorporated herein by reference).

A variety of methods are available for preparing liposomes for use within the invention (e.g., as described in Szoka et al., Ann. Rev. Biophys. Bioeng. 9:467, 1980; and U.S. Pat. Nos. 4,235,871, 4,501,728, and 4,837,028, each incorporated herein by reference). For use with liposome delivery, the dopamine receptor agonist and/or other biologically active agent is typically entrapped within the liposome, or lipid vesicle, or is bound to the outside of the vesicle. Several strategies have been devised to increase the effectiveness of liposome-mediated delivery by targeting liposomes to specific tissues and specific cell types. Liposome formulations, including those containing a cationic lipid, have been shown to be safe and well tolerated in human patients (Treat et al., J. Natl. Cancer Instit. 82:1706-1710, 1990, incorporated herein by reference).

Like liposomes, unsaturated long chain fatty acids, which also have enhancing activity for mucosal absorption, can form closed vesicles with bilayer-like structures (so called “ufasomes”). These can be formed, for example, using oleic acid to entrap dopamine receptor agonists, as well as biologically active peptides and proteins, for mucosal delivery within the invention.

Other delivery systems for use within the invention combine the use of polymers and liposomes seem to ally the advantageous properties of both vehicles. Exemplifying this type of hybrid delivery system, liposomes containing the model protein horseradish peroxidase (HRP) have been effectively encapsulated inside the natural polymer fibrin (Henschen et al., Blood Coagulation, pp. 171-241, Zwaal, et al., Eds., Elsevier, Amsterdam, 1986, incorporated herein by reference). Because of its biocompatibility and biodegradability, fibrin is a useful polymer matrix for drug delivery systems in this context (see, e.g., Senderoff, et al., J. Parenter. Sci. Technol. 45:2-6, 1991; and Jackson, Nat. Med.2:637-638, 1996, incorporated herein by reference). In addition, release of biotherapeutic compounds from this delivery system is controllable through the extent of covalent crosslinking and the addition of antifibrinolytic agents to the fibrin polymer (Uchino et al., Fibrinolysis 5:93-98, 1991, incorporated herein by reference).

More simplified delivery systems for use within the invention include the use of cationic lipids as delivery vehicles or carriers, which can be effectively employed to provide an electrostatic interaction between the lipid carrier and such charged biologically active agents (see, e.g., Hope et al., Molecular Membrane Biology 15:1-14, 1998, incorporated herein by reference). This allows efficient packaging of the drugs into a form suitable for mucosal administration and delivery to systemic compartments. These and related systems are particularly well suited for delivery of polymeric nucleic acids, e.g., in the form of gene constructs, antisense oligonucleotides and ribozymes. These drugs are large, usually negatively charged molecules with molecular weights on the order of 10₆ for a gene to 10₃ for an oligonucleotide. The targets for these drugs are intracellular, but their physical properties prevent them from crossing cell membranes by passive diffusion as with conventional drugs. Furthermore, unprotected DNA is degraded within minutes by nucleases present in normal plasma. To avoid inactivation by endogenous nucleases, antisense oligonucleotides and ribozymes can be chemically modified to be enzyme resistant by a variety of known methods, but plasmid DNA must ordinarily be protected by encapsulation in viral or non-viral envelopes, or condensation into a tightly packed particulate form by polycations such as proteins or cationic lipid vesicles. More recently, small unilamellar vesicles (SUVs) composed of a cationic lipid and dioleoylphosphatidylethanolamine (DOPE) have been successfully employed as vehicles for polynucleic acids, such as plasmid DNA, to form particles capable of transportation of the active polynucleotide across plasma membranes into the cytoplasm of a broad spectrum of cells. This process (referred to as lipofection or cytofection) is now widely employed as a means of introducing plasmid constructs into cells to study the effects of transient gene expression. Exemplary delivery vehicles of this type for use within the invention include cationic lipids (e.g., N-(2,3-(dioleyloxy)propyl)-N,N,N-trimethyl am-monium chloride (DOTMA)), quarternary ammonium salts (e.g., N,N-dioleyl-N, N-dimethylammonium chloride (DODAC)), cationic derivatives of cholesterol (e.g., 3β(N-(N′,N-dimethylaminoethane-carbamoyl-cholesterol (DC-chol)), and lipids characterized by multivalent headgroups (e.g., dioctadecyldimethylammonium chloride (DOGS), commercially available as Transfectam®).

Additional delivery vehicles for use within the invention include long and medium chain fatty acids, as well as surfactant mixed micelles with fatty acids (see, e.g., Muranishi, Crit. Rev. Ther. Drug Carrier Syst. 7:1-33, 1990, incorporated herein by reference). Most naturally occurring lipids in the form of esters have important implications with regard to their own transport across mucosal surfaces. Free fatty acids and their monoglycerides which have polar groups attached have been demonstrated in the form of mixed micelles to act on the intestinal barrier as penetration enhancers. This discovery of barrier modifying function of free fatty acids (carboxylic acids with a chain length varying from 12 to 20 carbon atoms) and their polar derivatives has stimulated extensive research on the application of these agents as mucosal absorption enhancers.

For use within the methods of the invention, long chain fatty acids, especially fusogenic lipids (unsaturated fatty acids and monoglycerides such as oleic acid, linoleic acid, linoleic acid, monoolein, etc.) provide useful carriers to enhance mucosal delivery of dopamine receptor agonists and other biologically active agents. Medium chain fatty acids (C6 to C12) and monoglycerides have also been shown to have enhancing activity in intestinal drug absorption and can be adapted for use within the intranasal delivery method of the invention. In addition, sodium salts of medium and long chain fatty acids are effective delivery vehicles and absorption-enhancing agents for intranasal delivery of biologically active agents within the invention. Thus, fatty acids can be employed in soluble forms of sodium salts or by the addition of non-toxic surfactants, e.g., polyoxyethylated hydrogenated castor oil, sodium taurocholate, etc. Mixed micelles of naturally occurring unsaturated long chain fatty acids (oleic acid or linoleic acid) and their monoglycerides with bile salts have been shown to exhibit absorption-enhancing abilities which are basically harmless to the intestinal mucosa (see, e.g., Muranishi, Pharm. Res. 2:108-118, 1985; and Crit. Rev. Ther. drug carrier Syst. 7:1-33, 1990, each incorporated herein by reference). Other fatty acid and mixed micellar preparations that are useful within the invention include, but are not limited to, Na caprylate (C8), Na caprate (C10), Na laurate (C12) or Na oleate (C18), optionally combined with bile salts, such as glycocholate and taurocholate.

PEGYLATION

Additional methods and compositions provided within the invention involve chemical modification of dopamine receptor agonists and, optionally, other biologically active molecules by covalent attachment of polymeric materials, for example dextrans, polyvinyl pyrrolidones, glycopeptides, polyethylene glycol and polyamino acids. The resulting conjugated active agents retain their biological activities and solubility for intranasal administration. In certain embodiments, dopamine receptor agonists or other molecules (e.g., biologically active peptides and proteins are conjugated to polyalkylene oxide polymers, particularly polyethylene glycols (PEG) (see, e.g., U.S. Pat. No. 4,179,337, incorporated herein by reference). Numerous reports in the literature describe the potential advantages of pegylated therapeutic compounds, which often exhibit increased resistance to enzymatic degradation, increased plasma half-life, increased solubility and decreased antigenicity and immunogenicity (Nucci, et al., Advanced Drug Deliver Reviews 6:133-155, 1991; Lu et al., Int. J. Peptide Protein Res. 43:127-138, 1994, each incorporated herein by reference). A number of proteins, including L-asparaginase, strepto-kinase, insulin, interleukin-2, adenosine deamidase, L-asparaginase, interferon alpha 2b, superoxide dismutase, streptokinase, tissue plasminogen activator (tPA), urokinase, uricase, hemoglobin, TGF-beta, EGF, and other growth factors, have been conjugated to PEG and evaluated for their altered biochemical properties as therapeutics (see, e.g., Ho, et al., Drug Metabolism and Disposition 14:349-352, 1986; Abuchowski et al., Prep. Biochem. 9:205-211, 1979; and Rajagopaian et al., J. Clin. Invest. 75:413-419, 1985, Nucci et al., Adv. Drug Delivery Rev. 4:133-151, 1991, each incorporated herein by reference). Although the in vitro biological activities of pegylated proteins may be decreased, this loss in activity is usually offset by the increased in vivo half-life in the bloodstream (Nucci, et al., Advanced Drug Deliver Reviews 6:133-155, 1991, incorporated herein by reference). Accordingly, these and other polymer-coupled therapeutic molecules within the invention exhibit enhanced properties, such as extended half-life and reduced immunogenicity, when administered mucosally according to the teachings herein.

Several procedures have been reported for the attachment of PEG to therapeutic compounds (e.g., proteins and peptides) and their subsequent purification (Abuchowski et al., J. Biol. Chem. 252:3582-3586,1977; Beauchamp et al., Anal. Biochem. 131:25-33, 1983, each incorporated herein by reference). For example, Lu et al., Int. J. Peptide Protein Res. 43:127-138, 1994 (incorporated herein by reference) describe various technical considerations and compare PEGylation procedures for proteins versus peptides (see also, Katre et al., Proc. Natl. Acad. Sci. USA 84:1487-1491, 1987; Becker et al., Makromol. Chem. Rapid Commun. 3:217-223, 1982; Mutter et al., Makromol. Chem. Rapid Commun. 13:151-157, 1992; Merrifield, R. B., J. Am. Chem. Soc. 85:2149-2154, 1993; Lu et al., Peptide Res. 6:142-146, 1993; Lee et al., Bioconjuzate Chem. 10:973-981, 1999, Nucci et al., Adv. Drug Deliv. Rev. 6:133-151, 1991; Francis et al., J. Drug Targeting 3:321-340, 1996; Zalipsky, S., Bioconiugate Chem. 6:150-165, 1995; Clark et al., J. Biol. Chem. 271:21969-21977, 1996; Pettit et al., J. Biol. Chem. 272:2312-2318, 1997; Delgado et al., Br. J. Cancer 73:175-182, 1996; Benhar et al., Bioconjugate Chem. 5:321-326, 1994; Benhar et al., J. Biol. Chem. 269:13398-13404, 1994; Wang et al., Cancer Res. 53:4588-4594, 1993; Kinstler et al., Pharm. Res. 13:996-1002, 1996, Filpula et al., Exp. Opin. Ther. Patents 9:231-245, 1999; Pelegrin et al., Hum. Gene Ther. 9:2165-2175, 1998, each incorporated herein by reference).

Following these and other teachings in the art, the conjugation of dopamine receptor agonists and other, optionalbiologically active peptides and proteins for with polyethyleneglycol polymers, is readily undertaken, with the expected result of prolonging circulating life and/or reducing immunogenicity while maintaining an acceptable level of activity of the PEGylated active agent. Amine-reactive PEG polymers for use within the invention include SC-PEG with molecular masses of 2000, 5000, 10000, 12000, and 20000; U-PEG-10000; NHS-PEG-3400-biotin; T-PEG-5000; T-PEG-12000; and TPC-PEG-5000. Chemical conjugation chemistries for these polymers have been published (see, e.g., Zalipsky, S., Bioconjugate Chem. 6:150-165, 1995; Greenwald et al., Bioconjugate Chem. 7:638-641, 1996; Martinez et al., Macromol. Chem. Phys. 198:2489-2498, 1997; Hermanson, G. T., Bioconjugate Techniques, pp. 605-618, 1996; Whitlow et al., Protein Eng. 6:989-995, 1993; Habeeb, A. F. S. A., Anal. Biochem. 14:328-336, 1966; Zalipsky et al., Poly(ethyleneglycol) Chemistry and Biological Applications, pp. 318-341, 1997; Harlow et al., Antibodies: a Laboratory Manual, pp. 553-612, Cold Spring harbor Laboratory, Plainview, N.Y., 1988; Milenic et al, Cancer Res. 51:6363-6371, 1991; Friguet et al., J. Immunol. Methods 77:305-319, 1985, each incorporated herein by reference). While phosphate buffers are commonly employed in these protocols, the choice of borate buffers may beneficially influence the PEGylation reaction rates and resulting products.

PEGylation of biologically active agents within the invention may be achieved by a variety of methods, for example by modification of carboxyl sites (e.g., aspartic acid or glutamic acid groups in addition to the carboxyl terminus). The utility of PEG-hydrazide in selective modification of carbodiimide-activated protein carboxyl groups under acidic conditions has been described (Zalipsky, S., Bioconjugate Chem. 6:150-165, 1995; Zalipsky et al., Poly(ethyleneglycol) Chemistry and Biological Applications, pp.318-341, American Chemical Society, Washington, D.C., 1997, incorporated herein by reference). Alternatively, bifunctional PEG modification of biologically active peptides and proteins can be employed. In some procedures, charged amino acid residues, including lysine, aspartic acid, and glutamic acid, have a marked tendency to be solvent accessible on protein surfaces. Conjugation to carboxylic acid groups of proteins is a less frequently explored approach for production of protein bioconjugates. However, the hydrazide/EDC chemistry described by Zalipsky and colleagues (Zalipsky, S., Bioconijugate Chem. 6:150-165, 1995; Zalipsky et al., Poly(ethyleneglycol) Chemistry and Biological Applications, pp. 318-341, American Chemical Society, Washington, D.C., 1997, each incorporated herein by reference) offers a practical method of linking PEG polymers to protein carboxylic sites. For example, this alternate conjugation chemistry has been shown to be superior to amine linkages for PEGylation of brain-derived neurotrophic factor (BDNF) while retaining biological activity (Wu et al., Proc. Natl. Acad. Sci. U.S.A. 96:254-259, 1999, incorporated herein by reference). Maeda and colleagues have also found carboxyl-targeted PEGylation to be the preferred approach for bilirubin oxidase conjugations (Maeda et al., Poly(ethylene glycol) Chemistry. Biotechnical and Biomedical Applications, J. M. Harris, Ed., pp. 153-169, Plenum Press, New York, 1992, incorporated herein by reference).

Often, PEGylation of biologically active agents for use within the invention involves activating PEG with a functional group that will react with lysine residues on the surface of the peptide or protein. Within certain alternate aspects of the invention, biologically active peptides and proteins are modified by PEGylation of other residues such as His, Trp, Cys, Asp, Glu, etc., without substantial loss of activity. If PEG modification of a selected peptide or protein proceeds to completion, the activity of the peptide or protein is often diminished. Therefore, PEG modification procedures herein are generally limited to partial PEGylation of the peptide or protein, resulting in less than about 50%, more commonly less than about 25%, loss of activity, while providing for substantially increased half-life (e.g., serum half life) and a substantially decreased effective dose requirement of the PEGylated active agent.

An unavoidable result of partial PEG modification is the production of a heterogenous mixture of PEGylated peptide or protein having a statistical distribution of the number of PEG groups bound per molecule. In addition, the usage of lysine residues within the peptide or protein is random. These two factors result in the production of a heterogeneous mixture of PEGylated proteins which differ in both the number and position of the PEG groups attached. For instance, when adenosine deaminase is optimally modified there is a loss of 50% activity when the protein has about 14 PEG per protein, with a broad distribution of the actual number of PEG moieties per individual protein and a broad distribution of the position of the actual lysine residues used. Such mixtures of diversely modified proteins are not optimally suited for pharmaceutical use. At the same time, purification and isolation of a class of PEGylated proteins (e.g., proteins containing the same number of PEG moieties) or a single type of PEGylated protein (e.g., proteins containing both the same number of moieties and having the PEG moieties at the same position) involves time-consuming and expensive procedures which result in an overall reduction in the yield of the specific PEGylated peptide or protein of interest.

Within certain alternate aspects of the invention, biologically active peptides and proteins are modified by PEGylation methods that employ activated PEG reagents that react with thio groups of the protein, resulting in covalent attachment of PEG to a cysteine residue, which residue may be inserted in place of a naturally-occurring lysine residue of the protein. As described, for example, in U.S. Pat. No. 5,166,322 (incorporated herein by reference) specific variants of IL-3 have been successfully produced which have a cysteine residue introduced at selected sites within the naturally occurring amino acid sequence. Sulfhydryl reactive compounds (e.g. activated polyethylene glycol) are then attached to these cysteines by reaction with the IL-3 variant. Additionally, U.S. Pat. No. 5,206,344 (incorporated herein by reference) describes specific IL-2 variants which contain a cysteine residue introduced at a selected sites within the naturally-occurring amino acid sequence. The IL-2 variant is subsequently reacted with an activated polyethylene glycol reagent to attach this moiety to a cysteine residue.

Yet additional methods employed within the invention for generating PEGylated peptides and proteins do not require extensive knowledge of protein structure-function (e.g., mapping amino acid residues essential for biological activity). Exemplifying these methods, U.S. Pat. No. 5,766,897 (incorporated herein by reference) describes methods for production and characterization of cysteine-PEGylated proteins suitable for therapeutic applications. These are produced by attaching a polyethylene glycol to a cysteine residue within the protein. To obtain the desired result of a stable, biologically active compound the PEG is attached in a specific manner, often to a cysteine residue present at or near a site which is normally glycosylated. Typically, the specific amino acid modified by glycosylation (e.g., asparagine in N-linked glycosylation or serine or threonine in O-linked glycosylation) is replaced by a cysteine residue, which is subsequently chemically modified by attachment of PEG. It may be useful for employment of this method to generation cysteine-containing mutants of selected biologically active peptides and proteins, which can be readily accomplished by, for example, site-directed mutagenesis using methods well known in the art (see, e.g., Kunkel, in Nucleic Acids and Molecular Biology, Eckstein, F. Lilley, D. M. J., eds., Springer-Verlag, Berling and Heidelberg, vol. 2, p. 124, 1988, incorporated herein by reference). In addition, if the active peptide or protein is one member of a family of structurally related proteins, glycosylation sites for any other member can be matched to an amino acid on the protein of interest, and that amino acid changed to cysteine for attachment of the polyethylene glycol. Alternatively, if a crystal structure has been determined for the protein of interest or a related protein, surface residues away from the active site or binding site can be changed to cysteine for the attachment of polyethylene glycol.

These strategies for identifying useful PEG attachment sites for use within the invention are advantageous in that they are readily implemented without extensive knowledge of protein structure-function details. Moreover, these strategies also take advantage of the fact that the presence and location of glycosylation residues are often related, as a natural evolutionary consequence, to increased stability and serum half-life of the subject peptide or protein. Replacement of these glycosylation residues by cysteine, followed by cysteine-specific PEGylation, commonly yields modified peptides and proteins which retain substantial biological activity while exhibiting significantly increased stability.

If a higher degree of PEG modification is required, and/or if the peptide or protein to be chemically modified is not normally glycosylated, other solvent accessible residues can be changed to cysteine, and the resultant protein subjected to PEGylation. Appropriate residues can easily be determined by those skilled in the art. For instance, if a three-dimensional structure is available for the protein of interest, or a related protein, solvent accessible amino acids are easily identified. Also, charged amino acids such as Lys, Arg, Asp and Glu are almost exclusively found on the surface of proteins. Substitution of one, two or many of these residues with cysteine will provide additional sites for PEG attachment. In addition, amino acid sequences in the native protein which are recognized by antibodies are usually on the surface of the protein. These and other methods for determining solvent accessible amino acids are well known to those skilled in the art.

Modification of biologically active agents with PEG can also be used to generate multimeric complexes which have increased biological stability and/or potency. For example, multimeric peptides and proteins may be naturally occurring dimeric or multimeric proteins. Dimeric peptides and proteins useful within the invention may be produced by reacting the peptide or protein with (Maleimido)₂-PEG, a reagent composed of PEG having two protein-reactive moieties. In the case of cysteine-pegylated peptides and proteins, the degree of multimeric cross-linking can be controlled by the number of cysteines either present and/or engineered into the peptide or protein, and by the concentration of reagents, e.g., (Maleimido)₂ PEG, used in the reaction mixture.

It is further contemplated to attach other groups to thio groups of cysteines present in biologically active peptides and proteins for use within the invention. For example, the peptide or protein may be biotinylated by attaching biotin to a thio group of a cysteine residue. Examples of cysteine-PEGylated proteins of the invention, as well as proteins having a group other than PEG covalently attached via a cysteine residue according to the invention, are as follows:

Other Stabilizing Modifications of Active Agents

In addition to PEGylation, dopamine receptor agonists and other biologically active agents, such as peptides and proteins, for use within the invention can be modified to enhance circulating half-life by shielding the active agent via conjugation to other known protecting or stabilizing compounds, for example by the creation of fusion proteins with an active peptide, protein, or analog linked to one or more carrier proteins, such as one or more immunoglobulin chains (see, e.g., U.S. Pat. Nos. 5,750,375; 5,843,725; 5,567,584 and 6,018,026, each incorporated herein by reference). These modifications will decrease the degradation, sequestration or clearance of the active agent and result in a longer half-life in a physiological environment (e.g., in the circulatory system, or at a mucosal surface). The active agents modified by these and other stabilizing conjugations methods are therefore useful with enhanced efficacy within the methods of the invention. In particular, the active agents thus modified maintain activity for greater periods at a target site of delivery or action compared to the unmodified active agent. Even when the active agent is thus modified, it retains substantial biological activity in comparison to a biological activity of the unmodified compound.

Thus, in certain aspects of the invention, biologically active agents for mucosal administration according to the methods of the invention are modified for enhanced activity, e.g., to increase circulating half-life, by shielding the active agent through conjugation to other known protecting or stabilizing compounds, or by the creation of fusion proteins with the peptide, protein or analog linked to one or more carrier proteins, such as one or more immunoglobulin chains (see, e.g., U.S. Pat. Nos. 5,750,375; 5,843,725; 5,567,584; and 6,018,026, each incorporated herein by reference). These modifications will decrease the degradation, sequestration or clearance of the active peptide or protein and result in a longer half-life in a physiological environment (e.g., at the nasal mucosal surface or in the systemic circulation). The active agents thus modified exhibit enhanced efficacy within the methods of the invention, for example by increased or temporally extended activity at a target site of delivery or action compared to the unmodified active agent.

In one aspect of the invention, active agents are conjugated for enhanced stability with relatively low molecular weight compounds, such as aminolethicin, fatty acids, vitamin B₁₂, and glycosides (see, e.g., Igarishi et al., Proc. Int. Symp. Control. Rel. Bioact. Materials, 17, 366, (1990). Additional exemplary modified active agents for use within the compositions and methods of the invention include known therapeutic agents modified for in vivo use by:

(a) chemical or recombinant DNA methods to link mammalian signal peptides (see, e.g., Lin et al., J. Biol. Chem. 270:14255, 1995, incorporated herein by reference) or bacterial peptides (see, e.g., Joliot et al., Proc. Natl. Acad. Sci. USA 88:1864, 1991, incorporated herein by reference) to the active peptide or protein, which serves to direct the active peptide or protein across cytoplasmic and organellar membranes and/or traffic the active peptide or protein to the a desired intracellular compartment (e.g., the endoplasmic reticulum (ER) of antigen presenting cells (APCs), such as dendritic cells for enhanced CTL induction);

(b) addition of a biotin residue to the active peptide or protein which serves to direct the active conjugate across cell membranes by virtue of its ability to bind specifically (i.e., with a binding affinity greater than about 10⁶, 10⁷, 10⁸, 10⁹, or 10¹⁰ M⁻¹) to a translocator present on the surface of cells (Chen et al., Analytical Biochem. 227:168, 1995, incorporated herein by reference);

(c) addition at either or both the amino- and carboxy-terminal ends of the active peptide or protein of a blocking agent in order to increase stability in vivo. This can be useful in situations in which the termini of the active peptide or protein tend to be degraded by proteases prior to cellular uptake or during intracellular trafficking. Such blocking agents can include, without limitation, additional related or unrelated peptide sequences that can be attached to the amino and/or carboxy terminal residues of the polypeptide or peptide to be administered. This can be done either chemically during the synthesis of the peptide or by recombinant DNA technology. Blocking agents such as pyroglutamic acid or other molecules known to those skilled in the art can be attached to the amino and/or carboxy terminal residues, or the amino group at the amino terminus or carboxyl group at the carboxy terminus can be replaced with a different moiety.

Biologically active agents modified by PEGylation and other stabilizing methods for use within the methods and compositions of the invention will preferably retain at least 25%, more preferably at least 50%, even more preferably between about 50% to 75%, most preferably 100% of the biological activity associated with the unmodified active agent, e.g., a native peptide or protein. Typically, the modified active agent, e.g., a conjugated peptide or protein, has a half-life (t_{1/2}), for example in serum following intranasal delivery, which is enhanced relative to the half-life of the unmodified active agent from which it was derived. In certain aspects, the half-life of a modified active agent for use within the invention is enhanced by at least 1.5-fold to 2-fold, often by about 2-fold to 3-fold, in other cases by about 5-fold to 10-fold, and up to 100-fold or more relative to the half-life of the unmodified active agent.

Prodrug Modifications

Yet another processing and formulation strategy useful within the invention is that of prodrug modification. By transiently (i.e., bioreversibly) derivatizing such groups as carboxyl, hydroxyl, and amino groups in small organic molecules, the undesirable physicochemical characteristics (e.g., charge, hydrogen bonding potential, etc. that diminish nasal mucosal penetration) of these molecules can be “masked” without permanently altering the pharmacological properties of the molecule. Bioreversible prodrug derivatives of therapeutic small molecule drugs has been shown to improve the physicochemical (e.g., solubility, lipophilicity) properties of numerous exemplary therapeutics, particularly those that contain hydroxyl and carboxylic acid groups.

One approach to making prodrugs of amine-containing active agents, such as peptides and proteins, is through the acylation of the amino group. Optionally, the use of acyloxyalkoxycarbamate derivatives of amines as prodrugs has been discussed. 3-(2′-hydroxy-4′,6′-dimethylphenyl)-3,3-dimethylpropionic acid has been employed to prepare linear, esterase-, phosphatase-, and dehydrogenase-sensitive prodrugs of amines (Amsberry et al., Pharm. Res. 8:455-461, 1991; Wolfe et al., J. Org. Chem. 57:6138, 1992, each incorporated herein by reference). These systems have been shown to degrade through a two step mechanism, with the first step being the slow, rate-determining enzyme-catalyzed (esterase, phosphatase, or dehydrogenase) step, and the second step being a rapid (t_{1/2}=100 sec., pH 7.4, 37° C.) chemical step (Amsberry et al., J. Org. Chem. 55:5867-5877, 1990, incorporated herein by reference). Interestingly, the phosphatase-sensitive system has recently been employed to prepare a very water-soluble (greater than 10 mg/ml) prodrug of TAXOL which shows significant antitumor activity in vivo. These and other prodrug modification systems and resultant therapeutic agents are useful within the methods and compositions of the invention.

For the purpose of preparing prodrugs of peptides that are useful within the invention, U.S. Pat. No. 5,672,584 (incorporated herein by reference) further describes the preparation and use of cyclic prodrugs of biologically active peptides and peptide nucleic acids (PNAs) To produce these cyclic prodrugs, the N-terminal amino group and the C-terminal carboxyl group of a biologically active peptide or PNA is linked via a linker, or the C-terminal carboxyl group of the peptide is linked to a side chain amino group or a side chain hydroxyl group via a linker, or the N-terminal amino group of said peptide is linked to a side chain carboxyl group via a linker, or a side chain carboxyl group of said peptide is linked to a side chain amino group or a side chain hydroxyl group via a linker. Useful linkers in this context include 3-(2′-hydroxy-4′,6′-dimethyl phenyl)-3,3-dimethyl propionic acid linkers and its derivatives, and acyloxyalkoxy derivatives. The incorporated disclosure provides methods useful for the production and characterization of cyclic prodrugs synthesized from linear peptides, e.g., opioid peptides that exhibit advantageous physicochemical features (e.g., reduced size, intramolecular hydrogen bond, and amphophilic characteristics) for enhanced cell membrane permeability and metabolic stability. These methods for peptide prodrug modification are also useful to prepare modified peptide therapeutic derivatives for use within the methods and compositions of the invention.

Purification and Preparation

Dopamine receptor agonists and other biologically active agents for mucosal administration according to the invention are generally provided for direct administration to subjects in a substantially purified form. The term “substantially purified” as used herein, is intended to refer to a compound that is isolated in whole or in part from naturally associated compounds and and other contaminants, wherein the active agent is purified to a measurable degree relative to its naturally-occurring state, e.g., relative to its purity within a cell extract.

In certain embodiments, the term “substantially purified” refers to a composition which has been subjected to fractionation to remove various contaminants, such as cell components. Of course, such purified preparations may include materials in covalent association with the active agent, such as glycoside residues or materials admixed or conjugated with the active agent, for example, to generate a modified derivative or analog of the active agent or produce a therapeutic formulation. The term purified thus includes variants wherein compounds such as polyethylene glycol, biotin or other moieties are bound to the active agent in order to allow for the attachment of other compounds and/or provide for formulations useful in therapeutic treatment or diagnostic procedures.

As applied to polynucleotides, the term substantially purified denotes that the polynucleotide is free of substances normally accompanying it, but may include additional sequence at the 5′ and/or 3′ end of the coding sequence which might result, for example, from reverse transcription of the noncoding portions of a message when the DNA is derived from a cDNA library, or might include the reverse transcript for the signal sequence as well as the mature protein encoding sequence.

When referring to peptides, proteins and peptide analogs (including peptide fusions with other peptides and/or proteins) of the invention, the term substantially purified typically means a composition which is partially to completely free of other cellular components with which the peptides, proteins or analogs are associated in a non-purified, e.g., native state or environment. Purified peptide is generally in a homogeneous state although it can be either in a dry state or in an aqueous solution. Purity and homogeneity are typically determined using analytical chemistry techniques such as polyacrylamide gel electrophoresis or high performance liquid chromatography.

Generally, substantially purified dopamine receptor agonists and other active compounds for use within the invention comprise more than 80% of all macromolecular species present in a preparation prior to admixture or formulation of the peptide or other active agent with a pharmaceutical carrier, excipient, buffer, absorption enhancing agent, stabilizer, preservative, adjuvant or other co-ingredient. More typically, the active agent is purified to represent greater than 90%, often greater than 95% of all macromolecular species present in a purified preparation. In other cases, the purified preparation of active agent may be essentially homogeneous, wherein other macromolecular species are not detectable by conventional techniques.

Various techniques suitable for use in purifying active agents for use within the invention are well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. Particularly useful purification methods include selective precipitation with such substances as ammonium sulfate; column chromatography; affinity methods, including immunopurification methods; and others (See, for example, R. Scopes, Protein Purification: Principles and Practice, Springer-Verlag: New York, 1982, incorporated herein by reference).

Peptides and proteins used in the methods and compositions of the invention can be obtained by a variety of means. Many peptides and proteins can be readily obtained in purified form from commercial sources. Smaller peptides (less than 100 amino acids long) can be conveniently synthesized by standard chemical methods familiar to those skilled in the art (e.g., see Creighton, Proteins: Structures and Molecular Principles, W. H. Freeman and Co., N.Y., 1983). Larger peptides (longer than 100 amino acids) can be produced by a number of methods including recombinant DNA technology (See, for example, the techniques described in Sambrook et al., Molecular Cloning A Laboratory Manual, Cold Spring Harbor Press, N.Y., 1989; and Ausubel et al., eds., Current Protocols in Molecular Biology, Green Publishing Associates, Inc., and John Wiley & Sons, Inc., N.Y, 1989, each incorporated herein by reference). Alternatively, RNA encoding the proteins can be chemically synthesized. See, for example, the techniques described in Oligonucleotide Synthesis, Gait, M. J., ed., IRL Press, Oxford, 1984 (incorporated herein by reference).

In certain embodiments of the invention, biologically active peptides or proteins will be constructed using peptide synthetic techniques, such as solid phase peptide synthesis (Merrifleld synthesis) and the like, or by recombinant DNA techniques, that are well known in the art. Peptide and protein analogs and mimetics will also be produced according to such methods. Techniques for making substitution mutations at predetermined sites in DNA include for example M13 mutagenesis. Manipulation of DNA sequences to produce substitutional, insertional, or deletional variants are conveniently described elsewhere such as Sambrook et al. (Molecular Cloning: A Laboratory Manual, Cold Spring Harbor Laboratories, Cold Spring Harbor, N.Y., 1989). In accordance with these and related teachings, defined mutations can be introduced into a biologically active peptide or protein to generate analogs and mimetics of interest by a variety of conventional techniques, e.g., site-directed mutagenesis of a cDNA copy of a portion of the gp120 gene encoding a selected peptide fragment, domain or motif. This can be achieved through and intermediate of single-stranded form, such as using the MUTA-gen® kit of Bio-Rad Laboratories (Richmond, Calif.), or a method using the double-stranded plasmid directly as a template such as the Chameleon® mutagenesis kit of Strategene (La Jolla, Calif.), or by the polymerase chain reaction employing either an oligonucleotide primer or a template which contains the mutation(s) of interest. A mutated subfragment can then be assembled into a complete peptide analog-encoding cDNA. A variety of other mutagenesis techniques are known and can be routinely adapted for use in producing mutations in biologically active peptides and proteins of interest for use within the invention.

In one method for obtaining purified active peptide or protein of interest, a polynucleotide molecule, for example a deoxyribonucleic acid (DNA) molecule, that defines a coding sequence for a peptide, protein, or peptide or protein analog is operably incorporated in a recombinant polynucleotide expression vector that directs expression of the peptide or analog in a suitable host cell. Exemplary methods for cloning and purifying peptides and analogs employing these novel polynucleotides and vectors are widely known in the art. Briefly, a polynucleotide encoding a selected peptide or protein is amplified by well known methods, such as the polymerase chain reaction (PCR). In this way the polynucleotide encoding the peptide or protein is obtained for expression and purification according to conventional methods. A DNA vector molecule that incorporates a DNA sequence encoding the subject peptide or protein can be operatively assembled, e.g., by linkage using appropriate restriction fragments from various plasmids which are described elsewhere. Also contemplated by the present invention are ribonucleic acid (RNA) equivalents of the above described polynucleotides comprising a coding sequence for a selected peptide or protein operatively linked in a polynucleotide expression construct for recombinant expression of the peptide or protein.

Once a polynucleotide molecule encoding an active peptide or protein is isolated and cloned, the peptide or protein can be expressed in a variety of recombinantly engineered cells. Numerous expression systems are available for expressing a DNA encoding a selected peptide. The expression of natural or synthetic nucleic acids encoding a biologically active peptide is typically achieved by operably linking the DNA to a promoter (which is either constitutive or inducible) within an expression vector. By expression vector is meant a polynucleotide molecule, linear or circular, that comprises a segment encoding the peptide of interest, operably linked to additional segments that provide for its transcription. Such additional segments include promoter and terminator sequences. An expression vector also may include one or more origins of replication, one or more selectable markers, an enhancer, a polyadenylation signal, etc. Expression vectors generally are derived from plasmid or viral DNA, and can contain elements of both. The term “operably linked” indicates that the segments are arranged so that they function in concert for their intended purposes, for example, transcription initiates in the promoter and proceeds through the coding segment to the terminator (see, e.g., Sambrook et al., Molecular Cloning: A Laboratory Manual, Cold Spring Harbor Laboratories, Cold Spring Harbor, N.Y., 1989, incorporated herein by reference).

Expression vectors can be constructed which contain a promoter to direct transcription, a ribosome binding site, and a transcriptional terminator. Examples of regulatory regions suitable for this purpose in E. coli are the promoter and operator region of the E. coli tryptophan biosynthetic pathway as described by Yanofsky, (J. Bacteriol, 158:1018-1024, 1984, incorporated herein by reference) and the leftward promoter of phage lambda (Pλ) as described by Herskowitz and Hagen (Ann. Rev. Genet. 14:399-445, 1980, incorporated herein by reference). The inclusion of selection markers in DNA vectors transformed in E. coli is also useful. Examples of such markers include genes specifying resistance to ampicillin, tetracycline, or chloramphenicol. Vectors used for expressing foreign genes in bacterial hosts generally will contain a selectable marker, such as a gene for antibiotic resistance, and a promoter which functions in the host cell. Plasmids useful for transforming bacteria include pBR322 (Bolivar, et al, Gene 2:95-113, 1977, incorporated herein by reference), the pUC plasmids (Messing, Meth. Enzymol. 101:20-77, 1983; Vieira and Messing, Gene 19:259-268. 1982, each incorporated herein by reference), pCQV2, and derivatives thereof. Plasmids may contain both viral and bacterial elements.

A variety of procaryotic expression systems can be used to express biologically active peptides and proteins for use within the invention. Examples include E. coli Bacillus, Streptomyces, and the like. Detection of the expressed peptide is achieved by methods such as radioimmunoassay, Western blotting techniques or immunoprecipitation. For expression in eukaryotes, host cells for use in practicing the invention include mammalian, avian, plant, insect, and fungal cells. Fungal cells, including species of yeast (e.g., Saccharomyces spp., Schizosaccharomyces spp.) or filamentous fungi (e.g., Aspergillus spp., Neurospora spp.) may be used as host cells within the present invention. Strains of the yeast Saccharomyces cerevisiae can be used. As explained briefly below, selected peptides and analogs can be expressed in these eukaryotic systems.

Suitable yeast vectors for use in the present invention include YRp7 (Struhl et al, Proc. Natl. Acad. Sci. USA 76:1035-1039, 1978, incorporated herein by reference), YEp13 (Broach et al., Gene 8:121-133, 1979, incorporated herein by reference), POT vectors (Kawasaki et al, U.S. Pat. No. 4,931,373, incorporated herein by reference), pJDB249 and pJDB219 (Beggs, Nature 275:104-108, 1978, incorporated herein by reference) and derivatives thereof. Such vectors generally include a selectable marker, which can be one of any number of genes that exhibit a dominant phenotype for which a phenotypic assay exists to enable transformants to be selected. Often, the selectable marker will be one that complements host cell auxotrophy, provides antibiotic resistance and/or enables a cell to utilize specific carbon sources, for example LEU2 (Broach et al., Gene 8:121-133, 1979), URA3 (Botstein et al., Gene 8:17, 1979, incorporated herein by reference), HIS3 (Struhl et al., Proc. Natl. Acad. Sci. USA 76:1035-1039, 1978) or POT1 (Kawasaki et al., U.S. Pat. No. 4,931,373). Another suitable selectable marker available for use within the invention is the CAT gene, which confers chloramphenicol resistance on yeast cells.

Examples of promoters for use in yeast include promoters from yeast glycolytic genes (Hitzeman et al., J. Biol. Chem. 255:12073-12080, 1980; Alber and Kawasaki, J. Mol. Appl. Genet. 1:419-434, 1982; Kawasaki, U.S. Pat. No. 4,599,311) or alcohol dehydrogenase genes (Young et al., Genetic Engineering of Microorganisms for Chemicals, Hollaender et al., eds., p. 355, Plenum, New York, 1982; Ammerer, Meth. Enzymol. 101:192-201, 1983). The TPI1 promoter (Kawasaki, U.S. Pat. No. 4,599,311) and the ADH2-4c promoter (Russell et al., Nature 304:652-654, 1983; and EP 284,044) also can be used. The expression units may also include a transcriptional terminator. An example of such a transcriptional terminator is the TPI1 terminator (Alber and Kawasaki, J. Mol. Appl. Genet. 1:419-434, 1982).

In addition to yeast, biologically active peptides and proteins of the present invention can be expressed in filamentous fungi, for example, strains of the fungi Aspergillus (McKnight et al., U.S. Pat. No. 4,935,349, which is incorporated herein by reference). Examples of useful promoters include those derived from Aspergillus nidulans glycolytic genes, such as the ADH3 promoter and the tpiA promoter. An example of a suitable terminator is the ADH3 terminator (McKnight et al., EMBO J. 4: 2093-2099, 1985, incorporated herein by reference). The expression units utilizing such components are cloned into vectors that are capable of insertion into the chromosomal DNA of Aspergillus.

Techniques for transforming fungi are well known in the literature, and have been described, for instance, by Beggs (Nature 275:104-108, 1978), Hinnen et al. (Proc. Natl. Acad. Sci. USA 75:1929-1933, 1978), Yelton et al. (Proc. Natl. Acad. Sci. USA 81:1740-1747, 1984), and Russell (Nature 301:167-169, 1983), each incorporated herein by reference. The genotype of the host cell generally contains a genetic defect that is complemented by the selectable marker present on the expression vector. Choice of a particular host and selectable marker is well within the level of ordinary skill in the art.

In addition to fungal cells, cultured mammalian cells can be used as host cells for expression of peptides and proteins useful within the present invention. Examples of cultured mammalian cells for use in the present invention include the COS-1 (ATCC CRL 1650), BHK, and 293 (ATCC CRL 1573; Graham et al., J. Gen. Virol. 6:59-72, 1977, incorporated herein by reference) cell lines. An example of a BHK cell line is the BHK 570 cell line (deposited with the American Type Culture Collection under accession number CRL 10314). In addition, a number of other mammalian cell lines can be used within the present invention, including rat Hep I (ATCC CRL 600), rat Hep II (ATCC CRL 1548), TCMK (ATCC CCL 139), human lung (ATCC CCL 75.1), human hepatoma (ATCC HTB-52), Hep G2 (ATCC HB 8065), mouse liver (ATCC CCL 29.1), NCTC 1469 (ATCC CCL 9.1) and DUKX cells (Urlaub and Chasin, Proc. Natl. Acad. Sci USA 77:4216-4220, 1980, incorporated herein by reference).

Mammalian expression vectors for use in expressing peptides and proteins useful within the invention include a promoter capable of directing the transcription of a cloned cDNA. Either viral promoters or cellular promoters can be used. Viral promoters include the immediate early cytomegalovirus (CMV) promoter (Boshart et al., Cell 41:521-530, 1985, incorporated herein by reference) and the SV40 promoter (Subramani et al., Mol. Cell. Biol. 1:854-864, 1981, incorporated herein by reference). Cellular promoters include the mouse metallothionein-1 promoter (Palmiter et al, U.S. Pat. No. 4,579,821, incorporated herein by reference), a mouse V [symbol] promoter (Bergman et al., Proc. Natl. Acad. Sci. USA 81:7041-7045, 1983; Grant et al., Nuc. Acids Res. 15:5496, 1987, each incorporated herein by reference), a mouse VH promoter (Loh et al., Cell 33:85-93, 1983, incorporated herein by reference), and the major late promoter from Adenovirus 2 (Kaufman and Sharp, Mol. Cell. Biol. 2:1304-13199, 1982, incorporated herein by reference).

Cloned DNA sequences can be introduced into cultured mammalian cells by, for example, calcium phosphate-mediated transfection (Wigler et al., Cell 14:725, 1978; Corsaro and Pearson, Somatic Cell Genetics 7:603, 1981; Graham and Van der Eb, Virology 52:456, 1973; each incorporated by reference herein in their entirety). Other techniques for introducing cloned DNA sequences into mammalian cells can also be used, such as electroporation (Neumann et al., EMBO J. 1:841-845, 1982, incorporated herein by reference) or cationic lipid-mediated transfection (Hawley-Nelson et al., Focus 15:73-79, 1993, incorporated herein by reference) using, e.g., a 3:1 liposome formulation of 2,3-dioleyloxy-N-[2 (sperminecarboxyamido)ethyl]-N,N-dimethyl-1-propanaminiumtrifluoroacetate and dioleoly-phosphatidylethanolamine in water Lipofectamine reagent, GIBCO-BRL). To identify cells that have integrated the cloned DNA, a selectable marker is generally introduced into the cells along with the gene or cDNA of interest. Examples of selectable markers for use in cultured mammalian cells include genes that confer resistance to drugs, such as neomycin, hygromycin, and methotrexate. The selectable marker can be an amplifiable selectable marker, for example the DHFR gene. Additional selectable markers are reviewed by Thilly (Mammalian Cell Technology, Butterworth Publishers, Stoneham, Mass., which is incorporated herein by reference). The choice of selectable markers is well within the level of ordinary skill in the art.

Selectable markers can be introduced into the cell on a separate plasmid at the same time as the polynucleotide encoding the selected peptide, or they may be introduced on the same plasmid. If on the same plasmid, the selectable marker and the peptide-encoding polynucleotide can be under the control of different promoters or the same promoter. Constructs of this latter type are known in the art (for example, Levinson and Simonsen, U.S. Pat. No. 4,713,339). It also can be advantageous to add additional DNA, known as “carrier DNA” to the mixture which is introduced into the cells.

Transfected mammalian cells are allowed to grow for a period of time, typically 1-2 days, to begin expressing the polynucleotide sequence(s) of interest. Drug selection is then applied to select for growth of cells that are expressing the selectable marker in a stable fashion. For cells that have been transfected with an amplifiable selectable marker the drug concentration is increased in a stepwise manner to select for increased copy number of the cloned sequences, thereby increasing expression levels.

Host cells containing polynucleotide constructs to direct expression of active peptides and protein are then cultured according to standard methods in a culture medium containing nutrients required for growth of the host cells. A variety of suitable media are known in the art and generally include a carbon source, a nitrogen source, essential amino acids, vitamins, minerals and growth factors. The growth medium generally selects for cells containing the DNA construct by, for example, drug selection or deficiency in an essential nutrient which is complemented by the selectable marker on the DNA construct or co-transfected with the DNA construct.

Recombinant peptides and proteins thus produced are purified by techniques well known to those of ordinary skill in the art. For example, the peptides or proteins can be directly expressed or expressed as fusion proteins. The proteins can then be purified by a combination of cell lysis (e.g., sonication) and affinity chromatography. For fusion products, subsequent digestion of the fusion protein with an appropriate proteolytic enzyme releases the desired peptide. Where the desired peptide or protein is soluble, it can be recovered from: (a) the culture, i.e., from the host cell in cases where the peptide or polypeptide is not secreted; or (b) from the culture medium in cases where the peptide or protein is secreted by the cells. Other expression systems comprise host cells that express a peptide or protein in situ, i.e., anchored in the cell membrane. Purification or enrichment of the peptide or protein from such an expression system can be accomplished using appropriate detergents and lipid micelles and methods well known to those skilled in the art.

Formulation and Administration

Mucosal delivery formulations of the present invention comprise the dopamine receptor agonist and, optionally, other biologically active agent to be administered, typically combined together with one or more pharmaceutically acceptable carriers and, optionally, other therapeutic ingredients. The carrier(s) must be “pharmaceutically acceptable” in the sense of being compatible with the other ingredients of the formulation and not deleterious to the subject. Such carriers are described herein above or otherwise well known to those skilled in the art of pharmacology. Desirably, the formulation should not include substances such as enzymes or oxidizing agents with which the biologically active agent to be administered is known to be incompatible. The formulations may be prepared by any of the methods well known in the art of pharmacy.

Compositions according to the present invention are often administered in an aqueous solution, e.g., as a nasal spray, and may be dispensed by a variety of methods known to those skilled in the art. Preferred systems for dispensing liquids as a spray are disclosed in U.S. Pat. No. 4,511,069. Such formulations may be conveniently prepared by dissolving compositions according to the present invention in water to produce an aqueous solution, and rendering said solution sterile. The formulations may be presented in multi-dose containers, for example in the sealed dispensing system disclosed in U.S. Pat. No. 4,511,069. Other suitable nasal spray delivery systems have been described in Transdermal Systemic Medication, Y. W. Chien Ed., Elsevier Publishers, New York, 1985; and in U.S. Pat. No. 4,778,810 (each incorporated herein by reference). Additional aerosol delivery forms may include, e.g., compressed air-, jet-, ultrasonic-, and piezoelectric nebulizers, which deliver the biologically active agent dissolved or suspended in a pharmaceutical solvent, e.g., water, ethanol, or a mixture thereof.

Nasal spray solutions of the present invention typically comprise the drug or drug to be delivered, optionally formulated with a surface active agent, such as a nonionic surfactant (e.g., polysorbate-80), and one or more buffers. In some embodiments of the present invention, the nasal spray solution further comprises a propellant. The pH of the nasal spray solution is optionally between about pH 6.8 and 7.2, but when desired the pH is adjusted to optimize delivery of a charged macromolecular species (e.g., a therapeutic protein or peptide) in a substantially unionized state. The pharmaceutical solvents employed can also be a slightly acidic aqueous buffer (pH 4-6). Suitable buffers for use within these compositions are as described above or as otherwise known in the art. Other components may be added to enhance or maintain chemical stability, including preservatives, surfactants, dispersants, or gases. Suitable preservatives include, but are not limited to, phenol, methyl paraben, paraben, m-cresol, thiomersal, benzylalkonimum chloride, and the like. Suitable surfactants include, but are not limited to, oleic acid, sorbitan trioleate, polysorbates, lecithin, phosphotidyl cholines, and various long chain diglycerides and phospholipids. Suitable dispersants include, but are not limited to, ethylenediaminetetraacetic acid, and the like. Suitable gases include, but are not limited to, nitrogen, helium, chlorofluorocarbons (CFCs), hydrofluorocarbons (HFCs), carbon dioxide, air, and the like.

Within alternate embodiments, mucosal formulations are administered as dry powder formulations comprising the dopamine receptor agonist and/or other biologically active agent in a dry, usually lyophilized, form of an appropriate particle size, or within an appropriate particle size range, for mucosal delivery. Minimum particle size appropriate for deposition within the nasal and pulmonary passages is often about 0.5μ°mass median equivalent aerodynamic diameter (MMEAD), commonly about 1μ MMEAD, and more typically about 2μ MMEAD. Maximum particle size appropriate for deposition within the nasal passages is often about 10μ MMEAD, commonly about 8μ MMEAD, and more typically about 4μ MMEAD. A particle size of about 3. Intranasally respirable powders within these size ranges can be produced by a variety of conventional techniques, such as jet milling, spray drying, solvent precipitation, supercritical fluid condensation, and the like. These dry powders of appropriate MMEAD can be administered to a patient via a conventional dry powder intranasal inhaler (DPI) which rely on the patient's breath, upon inhalation, to disperse the power into an aerosolized amount. Alternatively, the dry powder may be administered via air assisted devices that use an external power source to disperse the powder into an aerosolized amount, e.g., a piston pump.

Dry powder devices typically require a powder mass in the range from about 1 mg to 20 mg to produce a single aerosolized dose (“puff”). If the required or desired dose of the biologically active agent is lower than this amount, the powdered active agent will typically be combined with a pharmaceutical dry bulking powder to provide the required total powder mass. Preferred dry bulking powders include sucrose, lactose, dextrose, mannitol, glycine, trehalose, human serum albumin (HSA), and starch. Other suitable dry bulking powders include cellobiose, dextrans, maltotriose, pectin, sodium citrate, sodium ascorbate, and the like.

To formulate mucosal compositions for use within the present invention, the dopamine receptor agonist and/or other biologically active agent can be combined with various pharmaceutically acceptable additives, as well as a base or carrier for dispersion of the active agent(s). Desired additives include, but are not limited to, pH control agents, such as arginine, sodium hydroxide, glycine, hydrochloric acid, citric acid, etc. In addition, local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione) can be included. When the composition for mucosal delivery is a liquid, the tonicity of the formulation, as measured with reference to the tonicity of 0.9% (w/v) physiological saline solution taken as unity, is typically adjusted to a value at which no substantial, irreversible tissue damage will be induced in the nasal mucosa at the site of administration. Generally, the tonicity of the solution is adjusted to a value of about ⅓ to 3, more typically ½ to 2, and most often ¾ to 1.7.

The dopamine receptor agonist and/or other biologically active agent may be dispersed in a base or vehicle, which may comprise a hydrophilic compound having a capacity to disperse the active agent and any desired additives. The base may be selected from a wide range of suitable carriers, including but not limited to, copolymers of polycarboxylic acids or salts thereof, carboxylic anhydrides (e.g. maleic anhydride) with other monomers (e.g. methyl (meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters, etc. can be employed as carriers. Hydrophilic polymers and other carriers can be used alone or in combination, and enhanced structural integrity can be imparted to the carrier by partial crystallization, ionic bonding, crosslinking and the like. The carrier can be provided in a variety of forms, including, fluid or viscous solutions, gels, pastes, powders, microspheres and films for direct application to the nasal mucosa. The use of a selected carrier in this context may result in promotion of absorption of the dopamine receptor agonist and, optionally, other biologically active agent.

The dopamine receptor agonist and/or other biologically active agent can be combined with the base or carrier according to a variety of methods, and release of the active agent may be by diffusion, disintegration of the carrier, or associated formulation of water channels. In some circumstances, the active agent is dispersed in microcapsules (microspheres) or nanocapsules (nanospheres) prepared from a suitable polymer, e.g., isobutyl 2-cyanoacrylate (see, e.g., Michael et al., J. Pharmacy Pharmacol. 43: 1-5, 1991), and dispersed in a biocompatible dispersing medium applied to the mucosa, which yields sustained delivery and biological activity over a protracted time.

To further enhance mucosal delivery of pharmaceutical agents within the invention, formulations comprising the active agent may also contain a hydrophilic low molecular weight compound as a base or excipient. Such hydrophilic low molecular weight compounds provide a passage medium through which a water-soluble active agent, such as a physiologically active peptide or protein, may diffuse through the base to the body surface where the active agent is absorbed. The hydrophilic low molecular weight compound optionally absorbs moisture from the mucosa or the administration atmosphere and dissolves the water-soluble active peptide. The molecular weight of said hydrophilic low molecular weight compound is not more than 10000 and preferably not more than 3000. Exemplary hydrophilic low molecular weight compound include polyol compounds, such as oligo-, di- and monosaccarides such as sucrose, mannitol, lactose, L-arabinose, D-erythrose, D-ribose, D-xylose, D-mannose, D-galactose, lactulose, cellobiose, gentibiose, glycerin and polyethylene glycol. Other examples of hydrophilic low molecular weight compounds useful as carriers within the invention include N-methylpyrrolidone, and alcohols (e.g. oligovinyl alcohol, ethanol, ethylene glycol, propylene glycol, etc.) These hydrophilic low molecular weight compounds can be used alone or in combination with one another or with other active or inactive components of the mucosal formulation.

The compositions of the invention may alternatively contain as pharmaceutically acceptable carriers, substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

Therapeutic compositions for administering the dopamine receptor agonist and/or other biologically active agent can also be formulated as a solution, microemulsion, or other ordered structure suitable for high concentration of active ingredients. The carrier can be a solvent or dispersion medium containing, for example, water, ethanol, polyol (for example, glycerol, propylene glycol, and liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols such as mannitol, sorbitol, or sodium chloride in the composition. Prolonged absorption of the biologically active agent can be brought about by including in the composition an agent which delays absorption, for example, monostearate salts and gelatin.

In certain embodiments of the invention, the dopamine receptor agonist and/or other biologically active agent is administered in a time release formulation, for example in a composition which includes a slow release polymer. The active agent can be prepared with carriers that will protect against rapid release, for example a controlled release vehicle such as a polymer, microencapsulated delivery system or bioadhesive gel. Prolonged delivery of the active agent, in various compositions of the invention can be brought about by including in the composition agents that delay absorption, for example, aluminum monosterate hydrogels and gelatin. When controlled release formulations of the biologically active agent is desired, controlled release binders suitable for use in accordance with the invention include any biocompatible controlled-release material which is inert to the active agent and which is capable of incorporating the biologically active agent. Numerous such materials are known in the art. Useful controlled-release binders are materials which are metabolized slowly under physiological conditions following their mucosal delivery (e.g., at the nasal mucosal surface, or in the presence of bodily fluids following transmucosal delivery). Appropriate binders include but are not limited to biocompatible polymers and copolymers previously used in the art in sustained release formulations. Such biocompatible compounds are non-toxic and inert to surrounding tissues, and do not trigger significant adverse side effects such as nasal irritation, immune response, inflammation, or the like. They are metabolized into metabolic products which are also biocompatible and easily eliminated from the body.

Exemplary polymeric materials for use in this context include, but are not limited to, polymeric matrices derived from copolymeric and homopolymeric polyesters having hydrolysable ester linkages. A number of these are known in the art to be biodegradable and to lead to degradation products having no or low toxicity. Exemplary polymers include polyglycolic acids (PGA) and polylactic acids (PLA), poly(DL-lactic acid-co-glycolic acid)(DL PLGA), poly(D-lactic acid-coglycolic acid)(D PLGA) and poly(L-lactic acid-co-glycolic acid)(L PLGA). Other useful biodegradable or bioerodable polymers include but are not limited to such polymers as poly(epsilon-caprolactone), poly(epsilon-aprolactone-CO-lactic acid), poly(epsilon.-aprolactone-CO-glycolic acid), poly(beta-hydroxy butyric acid), poly(alkyl-2-cyanoacrilate), hydrogels such as poly(hydroxyethyl methacrylate), polyamides, poly(amino acids) (i.e., L-leucine, glutamic acid, L-aspartic acid and the like), poly (ester urea), poly (2-hydroxyethyl DL-aspartamide), polyacetal polymers, polyorthoesters, polycarbonate, polymaleamides, polysaccharides and copolymers thereof. Many methods for preparing such formulations are generally known to those skilled in the art (see, e.g., Sustained and Controlled Release Drug Delivery Systems, J. R. Robinson, ed., Marcel Dekker, Inc., New York, 1978, incorporated herein by reference). Other useful formulations include controlled-release compositions such as are known in the art for the administration of leuprolide (trade name: Lupron.RTM.), e.g., microcapsules (U.S. Pat. Nos. 4,652,441 and 4,917,893, each incorporated herein by reference), lactic acid-glycolic acid copolymers useful in making microcapsules and other formulations (U.S. Pat. Nos. 4,677,191 and 4,728,721, each incorporated herein by reference), and sustained-release compositions for water-soluble peptides (U.S. Pat. No. 4,675,189, incorporated herein by reference).

The mucosal formulations of the invention typically must be sterile and stable under all conditions of manufacture, storage and use. Sterile solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders, methods of preparation include vacuum drying and freeze-drying which yields a powder of the active ingredient plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

In more detailed aspects of the invention, the dopamine receptor agonist and/or other biologically active agent is stabilized to extend its effective half-life following delivery to the subject, particularly for extending metabolic persistence in an active state within the physiological environment (e.g., at the nasal mucosal surface, in the bloodstream, or within a connective tissue compartment or fluid-filled body cavity). For this purpose, the biologically active agent may be modified by chemical means, e.g., chemical conjugation, N-terminal capping, PEGylation, or recombinant means, e.g., site-directed mutagenesis or construction of fusion proteins, or formulated with various stabilizing agents or carriers. Thus stabilized, the active agent administered as above retains biological activity for an extended period (e.g., 2-3, up to 5-10 fold greater stability) under physiological conditions compared to its non-stabilized form.

In accordance with the various treatment methods of the invention, the dopamine receptor agonist and/or other biologically active agent is delivered to a mammalian subject in a manner consistent with conventional methodologies associated with management of the disorder for which treatment or prevention is sought. In accordance with the disclosure herein, a prophylactically or therapeutically effective amount of the dopamine receptor agonist and, optionally, other biologically active agent is administered to a subject in need of such treatment for a time and under conditions sufficient to prevent, inhibit, and/or ameliorate a selected disease or condition.

The term “subject” as used herein means any mammalian patient to which the compositions of the invention may be administered. Typical subjects intended for treatment with the compositions and methods of the present invention include humans, as well as non-human primates and other animals. To identify subject patients for prophylaxis or treatment according to the methods of the invention, accepted screening methods are employed to determine risk factors associated with a targeted or suspected disease of condition (e.g., sexual dysfunction, Parkinson's disease, etc.), or to determine the status of an existing disease or condition in a subject. These screening methods include, for example, conventional work-ups to determine familial, sexual, drug-use and other such risk factors that may be associated with the targeted or suspected disease or condition, as well as diagnostic methods such as various ELISA immunoassay methods, which are available and well known in the art to detect and/or characterize disease-associated markers (e.g., amyloid protein forms or HIV viral antigens). These and other routine methods allow the clinician to select patients in need of therapy using the mucosal methods and formulations of the invention. In accordance with these methods and principles, dopamine receptor agonists and other biologically active agents may be mucosally administered according to the teachings herein as an independent prophylaxis or treatment program, or as a follow-up, adjunct or coordinate treatment regimen to other treatments (for example to other anti-HIV treatments such as AZT and other anti-retroviral drug therapy), including surgery, vaccination, immunotherapy, hormone treatment, cell, tissue, or organ transplants, and the like.

Mucosal administration according to the invention allows effective self-administration of treatment by patients, provided that sufficient safeguards are in place to control and monitor dosing and side effects. Mucosal administration also overcomes certain drawbacks of other administration forms, such as injections, that are painful and expose the patient to possible infections and may present drug bioavailability problems. Systems for controlled aerosol dispensing of therapeutic liquids as a spray are well known. In one embodiment, metered doses of active agent are delivered by means of a specially constructed mechanical pump valve (U.S. Pat. No. 4,511,069, incorporated herein by reference). This hand-held delivery device is uniquely nonvented so that sterility of the solution in the aerosol container is maintained indefinitely.

Dosage

It is well known in the medical arts that dosages for human and other mammalian subjects depend on many factors. These subjective factors include, for exaple, the particular dop amine receptor agonist or other biologically active agent to be administered, the disease indication and particular status of the subject (e.g., the subject's age, size, fitness, extent of symptoms, susceptibility factors, etc), time and route of administration, and other drugs or treatments being administered concurrently. Dosages for peptide and protein therapeutics within the invention, including soluble antigens, will therefore vary, but can be approximately 0.01 mg to 100 mg per administration. Dosages for mucosal adjuvants will be approximately 0.001 mg to 100 mg per administration. Dosages for dopamine receptor agonists will typically be less than about 2 mg per administration. Dosages for cytokines, e.g., IL-12, will be approximately 25 μg/kg to 500 μg/kg per administration. Methods of determining optimal doses are well known to pharmacologists and physicians of ordinary skill. Thus, desired concentration of biologically active agents within the compositions of the present invention can be readily determined by those skilled in the art of pharmacology. These dosage determinations can be evaluated in animal models or human trials based on desired outcomes.

For prophylactic and treatment purposes, dopamine receptor agonists and other biologically active agents (e.g., immunogenic peptides, including anti-amyloid peptides) may be administered to the subject in a single bolus delivery, via continuous delivery (in an sustained release intranasal formulation) over an extended time period, or in a repeated administration protocol (e.g., on an hourly, daily or weekly basis). The various dosages and delivery protocols contemplated for administration of dopamine receptor agonists are therapeutically or prophylactically effective, at dosages and for periods of time necessary, to elicit an effective response in the subject, or to prevent or alleviate disease initiation or progression, or a related condition in the subject.

Dosage regimens may be adjusted to provide an optimal prophylactic therapeutic response. A therapeutically effective amount of a dopamine receptor agonist or other active agent is also one in which any toxic or detrimental side effects of the active agent is outweighed by therapeutically beneficial effects. A non-limiting range for a therapeutically effective amount of a biologically active agent within the invention is between about 0.01 μg/kg-0 mg/kg, more typically between about 0.05 and 5 mg/kg, and in certain embodiments between about 0.2 and 2 mg/kg. Dosages within this range can be achieved by single or multiple administrations, including, e.g., multiple administrations per day, daily or weekly administrations. Per administration, it is desirable to administer at least one microgram of a peptide or protein active agent, more typically between about 10 μg and 5.0 mg, and in certain embodiments between about 100 μg and 1.0 or 2.0 mg to an average human subject. It is to be further noted that for each particular subject, specific dosage regimens should be evaluated and adjusted over time according to the individual need and professional judgment of the person administering or supervising the administration of the biologically active agent. Dosage of the active agent may be varied by the attending clinician to maintain a desired concentration at the target site for drug action. For example, a predetermined desired concentration of the biologically active agent in the bloodstream may be between about 1-50 nanomoles per liter, sometimes between about 1.0 nanomole per liter and 10, 15 or 25 nanomoles per liter, depending on the subject's status and projected or measured response. Higher or lower concentrations may be selected based on the nature and stability of the active agent, and the content and method of the intranasal formulation. For example, dosage may be determined in part based on the release rate of the administered formulation, e.g., nasal spray versus powder, sustained release versus rapid-dissociation formulations, etc. To achieve the same serum concentration level, for example, slow-release particles with a release rate of 5 nanomolar (under standard conditions) would be administered at about twice the dosage of particles with a release rate of 10 nanomolar.

Additional guidance as to particular dosages for selected biologically active agents for use within the invention may be found widely disseminated in the literature. This is true for many of the particular peptide and protein reagents disclosed herein. For example, guidance for administration of human growth hormone (hGH) in the treatment of individuals intoxicated with poisonous substances may be found in U.S. Pat. Nos. 5,140,008 and 4,816,439; guidance for administration of hGH in the treatment of topical ulcers may be found in U.S. Pat. No. 5,006,509; guidance for administration of GM-CSF, G-CSF, and multi-CSF for treatment of pancytopenia may be found in U.S. Pat. No. 5,198,417; guidance for delivery of asparaginase for treatment of neoplasms may be found in U.S. Pat. Nos. 4,478,822 and 4,474,752; guidance for administration of L-asparaginase in the treatment of tumors is found in U.S. Pat. No. 5,290,773; guidance for administration of prostaglandin E1, prostaglandin E2, prostaglandin F2 alpha, prostaglandin 12, pepsin, pancreatin, rennin, papain, trypsin, pancrelipase, chymopapain, bromelain, chymotrypsin, streptokinase, urokinase, tissue plasminogen activator, fibrinolysin, deoxyribonuclease, sutilains, collagenase, asparaginase, or heparin in topical formulations may be found in U.S. Pat. No. 5,260,066; guidance for the administration of superoxide dismutase, glucocerebrosides, asparaginase, adenosine deaminase, interleukin (1,2,3,4,5,6,7), tissue necrosis factor (TNF-alpha or TNF-beta), and colony stimulating factors (CSF, G-CSF, GM-CSF) in liposomes may be found in U.S. Pat. No. 5,225,212; guidance for administration of asparaginase in the treatment of neoplastic lesions may be found in U.S. Pat. No. 4,978,332; guidance for administration of asparaginase in the reduction of tumor growth may be found in U.S. Pat. No. 4,863,910; guidance for the administration of antibodies in the prevention of transplant rejection may be found in U.S. Pat. Nos. 4,657,760 and 5,654,210; guidance for the administration of interleukin-1 as a therapy for immunomodulatory conditions including T cell mutagenesis, induction of cytotoxic T cells, augmentation of natural killer cell activity, induction of interferon-gamma, restoration or enhancement of cellular immunity, and augmentation of cell-mediated anti-tumor activity may be found in U.S. Pat. No. 5,206,344; guidance for the administration of interleukin-2 in the treatment of tumors may be found in U.S. Pat. No. 4,690,915; and guidance for administration of interleukin-3 in the stimulation of hematopoiesis, as a cancer chemotherapy, and in the treatment of immune disorders may be found in U.S. Pat. No. 5,166,322. Each of the foregoing U.S. patents is incorporated herein by reference with respect to the guidance provided for formulation and administration of particular biologically active agents therein).

Kits

The instant invention also includes kits, packages and multicontainer units containing the above described pharmaceutical compositions, active ingredients, and/or means for administering the same for use in the prevention and treatment of diseases and other conditions in mammalian subjects. Briefly, these kits include a container or formulation which contains a dopamine receptor agonist formulated in a pharmaceutical preparation for mucosal delivery. The dopamine receptor agonist is optionally contained in a bulk dispensing container or unit or multi-unit dosage form. Optional dispensing means may be provided, for example an intranasal spray applicator. Packaging materials optionally include a label or instruction which indicates that the dopamine receptor agonist packaged therewith can be used mucosally for treating or preventing a specific disease or condition (e.g., Parkinson's or erectile dysfunction). In more detailed embodiments of the invention, kits include a dopamine receptor agonist combined with one or more mucosal delivery-enhancing agents selected from: (a) aggregation inhibitory agents; (b) charge modifying agents; (c) pH control agents; (d) degradative enzyme inhibitors; (e) mucolytic or mucus clearing agents; (f) ciliostatic agents; (g) membrane penetration-enhancing agents (e.g., (i) a surfactant, (ii) a bile salt, (ii) a phospholipid or fatty acid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a clyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, (x) an inhibitor of cholesterol synthesis; or (xi) any combination of the membrane penetration enhancing agents of (i)-(x)); (h) modulatory agents of epithelial junction physiology, such as nitric oxide (NO) stimulators, chitosan, and chitosan derivatives; (i) vasodilator agents; (j) selective transport-enhancing agents; and (k) stabilizing delivery vehicles, carriers, supports or complex-forming species with which the dopamine receptor agonist(s) is/are effectively combined, associated, contained, encapsulated or bound for enhanced mucosal delivery.

The following examples are provided by way of illustration, not limitation.

### EXAMPLE I

Mucosal Delivery—Permeation Kinetics and Cytotoxicity

1. Organotypic Model

The following methods are generally useful for evaluating mucosal delivery parameters, kinetics and side effects for dopamine receptor agonists within the formulations and method of the invention, as well as for determining the efficacy and characteristics of the various mucosal delivery-enhancing agents disclosed herein for combinatorial formulation or coordinate administration with dopamine receptor agonists.

The EpiAirway system was developed by MatTek Corp (Ashland, Mass.) as a model of the pseudostratified epithelium lining the respiratory tract. The epithelial cells are grown on porous membrane-bottomed cell culture inserts at an air-liquid interface, which results in differentiation of the cells to a highly polarized morphology. The apical surface is ciliated with a microvillous ultrastructure and the epithelium produces mucus (the presence of mucin has been confirmed by immunoblotting). The inserts have a diameter of 0.875 cm, providing a surface area of 0.6 cm². The cells are plated onto the inserts at the factory approximately three weeks before shipping. One “kit” consists of 24 units.

a. On arrival, the units are placed onto sterile supports in 6-well microplates. Each well receives 5 mL of proprietary culture medium. This DMEM-based medium is serum free but is supplemented with epidermal growth factor and other factors. The medium is always tested for endogenous levels of any cytokine or growth factor which is being considered for intranasal delivery, but has been free of all cytokines and factors studied to date except insulin. The 5 mL volume is just sufficient to provide contact to the bottoms of the units on their stands, but the apical surface of the epithelium is allowed to remain in direct contact with air. Sterile tweezers are used in this step and in all subsequent steps involving transfer of units to liquid-containing wells to ensure that no air is trapped between the bottoms of the units and the medium.

b. The units in their plates are maintained at 37° C. in an incubator in an atmosphere of 5% CO₂ in air for 24 hours. At the end of this time the medium is replaced with fresh medium and the units are returned to the incubator for another 24 hours.

2. Experimental Protocol—Permeation Kinetics

a. A “kit” of 24 EpiAirway units can routinely be employed for evaluating five different formulations, each of which is applied to quadruplicate wells. Each well is employed for determination of permeation kinetics (4 time points), transepithelial resistance, mitochondrial reductase activity as measured by MTT reduction, and cytolysis as measured by release of LDH. An additional set of wells is employed as controls, which are sham treated during determination of permeation kinetics, but are otherwise handled identically to the test sample-containing units for determinations of transepithelial resistance and viability. The determinations on the controls are routinely also made on quadruplicate units, but occasionally we have employed triplicate units for the controls and have dedicated the remaining four units in the kit to measurements of transepithelial resistance and viability on untreated units or we have frozen and thawed the units for determinations of total LDH levels to serve as a reference for 100% cytolysis.

b. In all experiments, the mucosal delivery formulation to be studied is applied to the apical surface of each unit in a volume of 100 μL, which is sufficient to cover the entire apical surface. An appropriate volume of the test formulation at the concentration applied to the apical surface (no more than 100 μL is generally needed) is set aside for subsequent determination of concentration of the active material by ELISA or other designated assay.

c. The units are placed in 6 well plates without stands for the experiment: each well contains 0.9 mL of medium which is sufficient to contact the porous membrane bottom of the unit but does not generate any significant upward hydrostatic pressure on the unit.

d. In order to minimize potential sources of error and avoid any formation of concentration gradients, the units are transferred from one 0.9 mL-containing well to another at each time point in the study. These transfers are made at the following time points, based on a zero time at which the 100 μL volume of test material was applied to the apical surface: 15 minutes, 30 minutes, 60 minutes, and 120 minutes.

e. In between time points the units in their plates are kept in the 37° C. incubator. Plates containing 0.9 mL medium per well are also maintained in the incubator so that minimal change in temperature occurs during the brief periods when the plates are removed and the units are transferred from one well to another using sterile forceps.

f. At the completion of each time point, the medium is removed from the well from which each unit was transferred, and aliquotted into two tubes (one tube receives 700 μL and the other 200 μL) for determination of the concentration of permeated test material and, in the event that the test material is cytotoxic, for release of the cytosolic enzyme, lactic dehydrogenase, from the epithelium. These samples are kept in the refrigerator if the assays are to be conducted within 24 hours, or the samples are subaliquotted and kept frozen at −80° C. until thawed once for assays. Repeated freeze-thaw cycles are to be avoided.

g. In order to minimize errors, all tubes, plates, and wells are prelabeled before initiating an experiment.

h. At the end of the 120 minute time point, the units are transferred from the last of the 0.9 mL containing wells to 24-well microplates, containing 0.3 mL medium per well. This volume is again sufficient to contact the bottoms of the units, but not to exert upward hydrostatic pressure on the units. The units are returned to the incubator prior to measurement of transepithelial resistance.

3. Experimental Protocol—Transepithelial Resistance

a. Respiratory airway epithelial cells form tight junctions in vivo as well as in vitro, restricting the flow of solutes across the tissue. These junctions confer a transepithelial resistance of several hundred ohms×cm² in excised airway tissues; in the MatTek EpiAirway units, the transepithelial resistance (TER) is claimed by the manufacturer to be routinely around 1000 ohms x cm². We have found that the TER of control EpiAirway units which have been sham-exposed during the sequence of steps in the permeation study is somewhat lower (700-800 ohms×cm²), but, since permeation of small molecules is proportional to the inverse of the TER, this value is still sufficiently high to provide a major barrier to permeation. The porous membrane-bottomed units without cells, conversely, provide only minimal transmembrane resistance (5-20 ohms×cm²).

b. Accurate determinations of TER require that the electrodes of the ohmmeter be positioned over a significant surface area above and below the membrane, and that the distance of the electrodes from the membrane be reproducibly controlled. The method for TER determination recommended by MatTek and employed for all experiments here employs an “EVOM”™ epithelial voltohmmeter and an “ENDOHM”™ tissue resistance measurement chamber from World Precision Instruments, Inc.

c. The chamber is initially filled with Dulbecco's phosphate buffered saline (PBS) for at least 20 minutes prior to TER determinations in order to equilibrate the electrodes.

d. Determinations of TER are made with 1.5 mL of PBS in the chamber and 350 μL of PBS in the membrane-bottomed unit being measured. The top electrode is adjusted to a position just above the membrane of a unit containing no cells (but containing 350 μL of PBS) and then fixed to ensure reproducible positioning. The resistance of a cell-free unit is typically 5-20 ohms×cm² (“background resistance”).

e. Once the chamber is prepared and the background resistance is recorded, units in a 24-well plate which had just been employed in permeation determinations are removed from the incubator and individually placed in the chamber for TER determinations.

f. Each unit is first transferred to a petri dish containing PBS to ensure that the membrane bottom is moistened. An aliquot of 350 μL PBS is added to the unit and then carefully aspirated into a labeled tube to rinse the apical surface. A second wash of 350 μL PBS is then applied to the unit and aspirated into the same collection tube.

g. The unit is gently blotted free of excess PBS on its exterior surface only before being placed into the chamber (containing a fresh 1.5 mL aliquot of PBS). An aliquot of 350 μL PBS is added to the unit before the top electrode is placed on the chamber and the TER is read on the EVOM meter.

h. After the TER of the unit is read in the ENDOHM chamber, the unit is removed, the PBS is aspirated and saved, and the unit is returned with an air interface on the apical surface to a 24-well plate containing 0.3 mL medium per well.

i. The units are read in the following sequence: all sham-treated controls, followed by all formulation-treated samples, followed by a second TER reading of each of the sham-treated controls. After all the TER determinations are complete, the units in the 24-well microplate are returned to the incubator for determination of viability by MTT reduction.

4. Experimental Protocol—Viability by MTT Reduction

MTT is a cell-permeable tetrazolium salt which is reduced by mitochondrial dehydrogenase activity to an insoluble colored formazan by viable cells with intact mitochondrial function or by nonmitochondrial NAD(P)H dehydrogenase activity from cells capable of generating a respiratory burst. Formation of formazan is a good indicator of viability of epithelial cells since these cells do not generate a significant respiratory burst. We have employed a MTT reagent kit prepared by MatTek Corp for their units in order to assess viability.

a. The MTT reagent is supplied as a concentrate and is diluted into a proprietary DMEM-based diluent on the day viability is to be assayed (typically the afternoon of the day in which permeation kinetics and TER were determined in the morning). Insoluble reagent is removed by a brief centrifugation before use. The final MTT concentration is 1 mg/mL

b. The final MTT solution is added to wells of a 24-well microplate at a volume of 300 μL per well. As has been noted above, this volume is sufficient to contact the membranes of the EpiAirway units but imposes no significant positive hydrostatic pressure on the cells.

c. The units are removed from the 24-well plate in which they were placed after TER measurements, and after removing any excess liquid from the exterior surface of the units, they are transferred to the plate containing MTT reagent. The units in the plate are then placed in an incubator at 37° C. in an atmosphere of 5% CO₂ in air for 3 hours.

d. At the end of the 3-hour incubation, the units containing viable cells will have turned visibly purple. The insoluble formazan must be extracted from the cells in their units to quantitate the extent of MTT reduction. Extraction of the formazan is accomplished by transferring the units to a 24-well microplate containing 2 mL extractant solution per well, after removing excess liquid from the exterior surface of the units as before. This volume is sufficient to completely cover both the membrane and the apical surface of the units. Extraction is allowed to proceed overnight at room temperature in a light-tight chamber. MTT extractants traditionally contain high concentrations of detergent, and destroy the cells.

e. At the end of the extraction, the fluid from within each unit and the fluid in its surrounding well are combined and transferred to a tube for subsequent aliquotting into a 96-well microplate (200 μL aliquots are optimal) and determination of absorbance at 570 nm on a VMax multiwell microplate spectrophotometer. To ensure that turbidity from debris coming from the extracted units does not contribute to the absorbance, the absorbance at 650 nm is also determined for each well in the VMax and is automatically subtracted from the absorbance at 570 nm. The “blank” for the determination of formazan absorbance is a 200 μL aliquot of extractant to which no unit had been exposed. This absorbance value is assumed to constitute zero viability.

f. Two units from each kit of 24 EpiAirway units are left untreated during determination of permeation kinetics and TER. These units are employed as the positive control for 100% cell viability. In all the studies we have conducted, there has been no statistically significant difference in the viability of the cells in these untreated units vs cells in control units which had been sham treated for permeation kinetics and on which TER determinations had been performed. The absorbance of all units treated with test formulations is assumed to be linearly proportional to the percent viability of the cells in the units at the time of the incubation with MTT. It should be noted that this assay is carried out typically no sooner than four hours after introduction of the test material to the apical surface, and subsequent to rinsing of the apical surface of the units during TER determination.

5. Determination of Viability by LDH Release

While measurement of mitochondrial reductase activity by MTT reduction is a sensitive probe of cell viability, the assay necessarily destroys the cells and therefore can be carried out only at the end of each study. When cells undergo necrotic lysis, their cytotosolic contents are spilled into the surrounding medium, and cytosolic enzymes such as lactic dehydrogenase (LDH) can be detected in this medium. An assay for LDH in the medium can be performed on samples of medium removed at each time point of the two-hour determination of permeation kinetics. Thus, cytotoxic effects of formulations which do not develop until significant time has passed can be detected as well as effects of formulations which induce cytolysis with the first few minutes of exposure to airway epithelium.

a. The recommended LDH assay for evaluating cytolysis of the EpiAirway units is based on conversion of lactate to pyruvate with generation of NADH from NAD. The NADH is then reoxidized along with simultaneous reduction of the tetrazolium salt INT, catalyzed by a crude “diaphorase” preparation. The formazan formed from reduction of INT is soluble, so that the entire assay for LDH activity can be carried out in a homogenous aqueous medium containing lactate, NAD, diaphorase, and INT.

b. The assay for LDH activity is carried out on 50 μL aliquots from samples of “supernatant” medium surrounding an EpiAirway unit and collected at each time point. These samples were either stored for no longer than 24 h in the refrigerator or were thawed after being frozen within a few hours after collection. Each EpiAirway unit generates samples of supernatant medium collected at 15 min, 30 min, 1 h, and 2 h after application of the test material. The aliquots are all transferred to a 96 well microplate.

c. A 50 μL aliquot of medium which had not been exposed to a unit serves as a “blank” or negative control of 0% cytotoxicity. We have found that the apparent level of “endogenous” LDH present after reaction of the assay reagent mixture with the unexposed medium is the same within experimental error as the apparent level of LDH released by all the sham-treated control units over the entire time course of 2 hours required to conduct a permeation kinetics study. Thus, within experimental error, these sham-treated units show no cytolysis of the epithelial cells over the time course of the permeation kinetics measurements.

d. To prepare a sample of supernatant medium reflecting the level of LDH released after 100% of the cells in a unit have lysed, a unit which had not been subjected to any prior manipulations is added to a well of a 6-well microplate containing 0.9 mL of medium as in the protocol for determination of permeation kinetics, the plate containing the unit is frozen at −80° C., and the contents of the well are then allowed to thaw. This freeze-thaw cycle effectively lyses the cells and releases their cytosolic contents, including LDH, into the supernatant medium. A 50 μL aliquot of the medium from the frozen and thawed cells is added to the 96-well plate as a positive control reflecting 100% cytotoxicity.

e. To each well containing an aliquot of supernatant medium, a 50 μL aliquot of the LDH assay reagent is added. The plate is then incubated for 30 minutes in the dark.

f. The reactions are terminated by addition of a “stop” solution of 1 M acetic acid, and within one hour of addition of the stop solution, the absorbance of the plate is determined at 490 nm.

g. Computation of percent cytolysis is based on the assumption of a linear relationship between absorbance and cytolysis, with the absorbance obtained from the medium alone serving as a reference for 0% cytolysis and the absorbance obtained from the medium surrounding a frozen and thawed unit serving as a reference for 100% cytolysis.

6. ELISA Determinations

The procedures for determining the concentrations of active test material which have permeated the epithelial cells into the surrounding medium over the multiple time points are generally as described by the manufacturers of the specific ELISA kits employed for assay. These kits are typically two-step sandwich ELISAs: the immunoreactive form of the agent being studied is first “captured” by an antibody immobilized on a 96-well microplate and after washing unbound material out of the wells, a “detection” antibody is allowed to react with the bound immunoreactive agent. This detection antibody is typically conjugated to an enzyme (most often horseradish peroxidase) and the amount of enzyme bound to the plate in immune complexes is then measured by assaying its activity with a chromogenic reagent. In addition to samples of supernatant medium collected at each of the time points in the permeation kinetics studies, appropriately diluted samples of the formulation which was applied to the apical surface of the units at the start of the kinetics study are also assayed in the ELISA plate, along with a set of manufacturer-provided standards. Each supernatant medium sample is generally assayed in duplicate wells by ELISA (it will be recalled that quadruplicate units are employed for each formulation in a permeation kinetics determination, generating a total of sixteen samples of supernatant medium collected over all four time points).

a. It is not uncommon for the apparent concentrations of active test agent in samples of supernatant medium or in diluted samples of material applied to the apical surface of the units to lie outside the range of concentrations of the standards after completion of an ELISA. No concentrations of material present in experimental samples are determined by extrapolation beyond the concentrations of the standards; rather, samples are rediluted appropriately to generate concentrations of the test material which can be more accurately determined by interpolation between the standards in a repeat ELISA.

b. The ELISA for human growth hormone (hGH) is unique in its design and recommended protocol. Unlike most kits, the hGH ELISA employs two monoclonal antibodies, one for capture and another, directed towards a nonoverlapping hGH determinant, as the detection antibody (this antibody is conjugated to horseradish peroxidase). As long as concentrations of hGH which lie below the upper limit of the assay are present in experimental samples, the assay protocol can be employed as per the manufacturer's instructions, which allow for incubation of the samples on the ELISA plate with both antibodies present simultanously. When the hGH levels in a sample are significantly higher than this upper limit, the levels of immunoreactive hGH may exceed the amounts of the antibodies in the incubation mixture, and some hGH which has no detection antibody bound will be captured on the plate, while some hGH which has detection antibody bound may not be captured. This leads to serious underestimation of the hGH levels in the sample (it will appear that the hGH levels in such a sample lie significantly below the upper limit of the assay). To eliminate this possibility, the assay protocol has been modified:

b.1. The diluted samples are first incubated on the ELISA plate containing the immobilized capture antibody for one hour in the absence of any detection antibody. After the one hour incubation, the wells are washed free of unbound material.

b.2. The detection antibody is incubated with the plate for one hour to permit formation of immune complexes with all captured antigen. The concentration of detection antibody is sufficient to react with the maximum level of hGH which has been bound by the capture antibody. The plate is then washed again to remove any unbound detection antibody.

b.3. The peroxidase substrate is added to the plate and incubated for fifteen minutes to allow color development to take place.

p.4. The “stop” solution is added to the plate, and the absorbance is read at 450 as well as 490 nm in the VMax microplate spectrophotometer. The absorbance of the colored product at 490 nm is much lower than that at 450 nm, but the absorbance at each wavelength is still proportional to concentration of product. The two readings ensure that the absorbance is linearly related to the amount of bound hGH over the working range of the VMax instrument (we routinely restrict the range from 0 to 2.5 OD, although the instrument is reported to be accurate over a range from 0 to 3.0 OD). The amount of hGH in the samples is determined by interpolation between the OD values obtained for the different standards included in the ELISA. Samples with OD readings outside the range obtained for the standards are diluted and run in a repeat ELISA.

### EXAMPLE II

Exemplary Formulations of Apomorphine for Enhanced Mucosal Delivery

An exemplary formulation for enhanced mucosal delivery of apomorphine following the teachings of the instant specification was prepared and evaluated as follows:

Formulation Composition

| # | Items | % w/w |
|---|---|---|
| 1 | Apomorphine HCL, USP | 0.25 or 0.50 |
| 2 | Citric Acid Anhydrous, USP | 0.68 |
| 3 | Sodium Citrate Dihydrate, USP | 0.44 |
| 4 | Propylene Glycol, USP | 7.0 |
| 5 | Glycerin, USP | 5.0 |
| 6 | L-Ascorbic Acid, USP | 0.012 |
| 7 | Sodium Metabisulfite, NF | 0.088 |
| 8 | Edetate Disodium, USP | 0.02 |
| 9 | Benzalkonium Chloride, NF (50% Soln) | 0.04 |
| 10 | Sodium Hydroxide, NF or Hydrochloric | To adjust pH to pH 3.5 |
| | Acid, NF | |
| 11 | Purified Water, USP (qs) | to 100 ml |

This exemplary formulation was demonstrated to exhibit greatly enhanced stability (marked by a clear to yellow solution color) compared to the Illum and Merkus et al. formulations (described further above). In particular, this formulation exhibited stability at an “accelerated” temperature storage condition of 40° C. for up to 30 weeks. The apomorphine concentration can be varied to allow delivery of between about 0.25 mg and about 2.0 mg with each spray.

Additional exemplary formulations were made and tested which demonstrated that a pH of about 3.0 also provides for a highly stabilized formulation of apomorphine for intranasal delivery (even when formulated with only a single reducing agent—sodium metabisulfite). Two such exemplary formulations (pH 3.07, and pH 3.01; formulated with apomorphine, metabisulfite, EDTA, and polysorbate, and without ascorbate, citric acid, sodium laurate, benzalkonium chloride, propylene glycol or sodium hydroxide) exhibited stability (marked by a clear to yellow solution color) at an “accelerated” temperature storage condition of 40° C. for up to 17 and 23 weeks, respectively. Additional comparative formulations and results obtained for formulations having a pH outside of the claimed range (exemplary outside pH values tested in this context included pH 4.45, 4.84, 4.95, and 5.01) demonstrate that a preferred pH range of about pH 3.0 to about pH 3.5 provides for an unexpectedly significant degree of stabilization of the dopamine receptor agonist in the claimed formulations, independent of other factors including the presence or absence of multiple reducing agents. For additional disclosure regarding the effects of pH, and the relative efficacy of different concentrations of apomorphine in mucosal formulations, see, e.g., copending U.S. patent application Ser. No. 09/334,304, filed Jun. 16, 1999 (and its corresponding priority U.S. Provisional Application Serial No. 60/096,545, filed Aug. 14, 1998 and corresponding PCT Publication WO 00/76509, published Dec. 21, 2000); and U.S. patent application Ser. No. 09/665,500, filed Sep. 19, 2000, each incorporated herein by reference. These incorporated disclosures also describe the utility of providing a plurality of reducing agents to stabilize apomorphine formulations, which is disclosed herein to involve a coupled redox protection of apomorphine against oxidation by coupling apomorphine as a redox exchange substrate or catalyst between two reducing agents having different redox potentials—one of which is greater and one of which is lesser than redox potentials for two alternating redox states of apomorphine in the redox-coupled reaction.

The use of a plurality of reducing agents inan apomorphine formulation (i.e., two or more reducing agents exemplified by sodium ascorbate, ascorbic acid, and sodium metabisulfite) provides superior stabilization compared to stabilization that is achieved using only a single reducing agent (e.g., sodium metabisulfite only). This increased stabilization is unexpected in the sense that reducing agents would generally not be predicted to have an additive or synergistic effect beyond mere additive concentration effects. Also it is generally counterintuitive to use multiple reagents having reducing activity in pharmaceutical formulations, unless their activities were otherwise predicted to be differentially advantageous. Using multiple ingredients with a common activity would unnecessarily increase the complexity and cost of preparing the formulation, and would also increase the likelihood of problems in using the formulation (such as incompatibility of ingredients and other quality control problems, potential for adverse side effects, etc.) One skilled in the art, without further information, would therefore ordinarily select only one such ingredient having the best activity or degree of characterization among known ingredients to achieve the desired (e.g., reducing) effect, which would generally be viewed as the most reliable way to optimize the formulation.

### EXAMPLE IV

Enhanced Mucosal Delivery of Apomorphine into the Cerebral Spinal Fluid of Human Subjects

The formulation of Example II, above, was administered in the following study:

STUDY SYNOPSIS. The present example provides a non-blinded study to determine the uptake of intranasally administered apomorphine hydrochloride into the cerebrospinal fluid (C SF) in healthy male volunteers. The study involved administration of apomorphine hydrochloride nasal formulation, as described above.

Six healthy male subjects, ages 18-40, were enrolled in the study. Each subject received a single dose of intranasal apomorphine hydrochloride. Subsequently, each subject underwent lumbar puncture, with the retrieval of 4.0 mL of CSF (4 tubes, 1.0 mL per tube). CSF samples were obtained at 20 minute time point post dosing.

The cerebrospinal fluid was evaluated for total apomorphine hydrochloride content, as well as glucose, protein, and cell count.

The specific objectives of this study were to obtain cerebrospinal fluid levels of apomorphine hydrochloride from six healthy male volunteers each of whom have received apomorphine hydrochloride intranasally.

The intent of the study, the study protocol, and the Informed Consent Form to be used in the study was approved in writing by the IRB prior to initiation of the study.

Subject Inclusion Criteria. Healthy, non-smoking (greater than 6 months), male volunteers, ages 18-40, were drawn from the population at large. Medical histories, physical examinations, and ancillary screenings were performed. Demographic data, subject initials, gender, age, weight, height, body build and statement of non-smoking status were recorded. The male subjects had a normal nasal mucosa. The male subjects read, signed and received a copy of the Informed Consent Form prior to initiation of any study procedure.

Subject Exclusion Criteria. The following exclusion criteria were used:

Any significant underlying medical pathology, as determined by history, physical examination, or ancillary pre-trial testing

Known hypersensitivity or idiosyncratic response to apomorphine

History of epistaxis or allergic rhinitis

Sulfite allergy

Any history of antecedent back surgery

Any history of signs or symptoms consistent with a diagnosis of lumbosacral radiculopathy

Known or suspected bleeding diathesis

Recent ingestion of any non-steroidal anti-inflammatory agents

Anti-coagulation of any type

History of alcoholism or drug abuse

Any significant psychiatric disorder

Any subject may be excluded at the discretion of the Principal Investigator, on an historical, clinical, or ancillary basis

Treatment Plan. Subjects were instructed to refrain from strenuous exertion activity for a minimum of three hours prior to testing. Also, they were instructed to refrain from all prescription, non-prescription, and holistic therapies for a minimum of three days prior to testing and antibiotics for at least two days.

When receiving the intranasal formulations, subjects were seated and instructed to gently blow their nose prior to dosing. During intranasal dosing, the contralateral nostril was occluded, with pressure applied by the subject's forefinger. Subjects remained in a seated position, with head upright, for 5 minutes post-dosing. Subjects informed the clinician if they sneeze or if the product drips from their nostril(s). The on-site clinician or designee using a metered-dose nasal applicator administered the doses.

After having been given informed consent, subjects were administered apomorphine hydrochloride, at a dose of 0.25-0.50 mg, in an intranasal formulation. Subsequently, the investigator positioned the patient appropriately in order to proceed with lumbar puncture.

The lumbar area was prepared and draped in the usual aseptic fashion. Local anesthesia was utilized (1% Xylocaine (lidocaine)), 1-5 mL, to be obtained from a commercial distributor). Upon adequate anesthesia, a spinal needle (20 or 22G) was introduced into the spinal canal, at the level deemed appropriate by the Investigator. No indwelling CSF catheters were used. The CSF samples were withdrawn 10 or 20 minutes after the administration of the nasal apomorphine. A total of 4.0 mL of CSF were collected from each patient, and distributed into 4 separate collection tubes. The tubes were appropriately labeled with a patient identifier and submitted for bioanalytical analysis.

Upon completion of CSF collection, the spinal needle was removed. The area was cleaned with antiseptic solution, and a sterile dressing was applied. Afterward, the subjects were required to rest in a supine position for an observation period. The subjects were offered food and drink during this time period.

The confinement extended for approximately 4 hours after completion of the specimen collection and removal of the spinal catheter, and concluded at the discretion of the Principal Investigator. A final set of vital signs were obtained and recorded prior to subject discharge.

Subjects were instructed to refrain from any significant physical activity for the ensuing 48 hours. Specific written instructions were supplied. A follow-up by telephone call with each subject within 24-48 hours upon completion of the procedure was performed.

The investigator retained a frozen sample of each subjects CSF. Apomorphine concentrations in the CSF will be measured by LC-MS-MS. All laboratory analysis for apomorphine concentration will be performed by Keystone Analytical Laboratories, Inc., 113 Dickerson Road, Unit 6, North Wales, Pa. 19454.

The results of the clinical study described above were as follows:

TABLE 1

| | CSF level at | Plasma level at | |
|---|---|---|---|
| | 20 min | 20 min | CSF to Plasma ratio |
| Subject # | ng/mL | ng/mL | (%) |
| 1 | 0.115 | 0.323 | 35.6% |
| 2 | 0.115 | 0.261 | 44.1% |
| 3 | 0.072 | 0.270 | 26.7% |
| 4 | 0.080 | 0.283 | 28.3% |
| 5 | 0.099 | 0.250 | 39.6% |
| Prior Art: | | | |
| SC Inj (ref. 1) | 1.08 | 25.04 | 4.3% |
| SC Inj (ref. 2) | | | 2.5 to 3.6% |

Reference 1 (involving 6 patients): Przedborski et al., Mov. Disord. 10: 28-36, 1995.
Reference 2 (involving 2 patients): Hofstee et al. Clin. Neuropharmacol. 17: 45-52, 1994

As can be seen by the data shown above in Table 1, while the prior art formulations provide 2.5 to 4.3% levels in the cerebral spinal fluid compared to the plasma, the exemplary formulation of the instant invention provided CNS levels of 26.7% to 44.1% relative to plasma levels under comparable experimental conditions. In fact, the formulations of the instant invention provide an average apomporphine concentration at the times indicated of 34.9%, with a standard deviation of 7%, to the CSF relative to the plasma concentration. Thus, a value 4.3% for the prior art formulations is over four standard deviations below the formulations in the instant example. These results are surprising and fully consistent with the foregoing disclosure, and no clinically significant adverse side effects were observed during the study.

Additional advantages and modifications of the invention disclosed herein will occur to those persons skilled in the art. Accordingly, the invention in its broader aspects is not limited to the specific details or illustrated examples described herein. Therefore, all departures made from the detail are deemed to be within the scope of the invention as defined by the appended claims.

What is claimed is:

1. A stable pharmaceutical formulation comprising a dopamine receptor agonist and one or more delivery-enhancing agent(s), wherein said formulation following mucosal adminstration to a mammalian subject yields a peak concentration of said dopamine receptor agonist in a central nervous system tissue or fluid of said subject that is 5% or greater compared to a peak concentration of said dopamine receptor agonist in a blood plasma of said subject.

2. The formulation of claim 1, wherein said mucosal administration involves delivery of said formulation to a nasal mucosal surface of said subject.

3. The formulation of claim 1, wherein the dopamine receptor agonist is apomorphine or a pharmaceutically acceptable salt or derivative thereof.

4. The formulation of claim 1, wherein said dopamine receptor agonist is administered to said subject in an effective dose of between about 0.25 and 2.0 mg.

5. The formulation of claim 1, wherein said delivery-enhancing agent(s) is/are selected from:
(a) an aggregation inhibitory agent;
(b) a charge modifying agent;
(c) a pH control agent;
(d) a degradative enzyme inhibitory agent;
(e) a mucolytic or mucus clearing agent;
(f) a ciliostatic agent;
(g) a membrane penetration-enhancing agent selected from (i) a surfactant, (ii) a bile salt, (ii) a phospholipid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a clyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, or (x) an inhibitor of cholesterol synthesis; or (xi) any combination of the membrane penetration enhancing agents recited in (i)-(x);
(h) a modulatory agent of epithelial junction physiology;
(i) a vasodilator agent;
(j) a selective transport-enhancing agent; and
(k) a stabilizing delivery vehicle, carrier, support or complex-forming species with which the dopamine receptor agonist is effectively combined, associated, contained, encapsulated or bound resulting in stabilization of the dopamine receptor agonist for enhanced mucosal delivery, wherein the formulation of said dopamine receptor agonist with said one or more delivery-enhancing agents provides for increased bioavailability of the dopamine receptor agonist in a central nervous system tissue or fluid of said subject.

6. The formulation of claim 1, wherein said delivery-enhancing agent(s) is/are selected from the group consisting of citric acid, sodium citrate, propylene glycol, glycerin, L-ascorbic acid, sodium metabisulfite, edetate disodium, benzalkonium chloride, sodium hydroxide and mixtures thereof.

7. The formulation of claim 1, wherein said formulation mucosally administered to said subject yields a peak dopamine receptor agonist concentration in a cerebral spinal fluid of said subject that is between about 5-10% of the peak dopamine receptor agonist concentration in the blood plasma of said subject.

8. The formulation of claim 1, wherein said formulation mucosally administered to said subject yields a peak dopamine receptor agonist concentration in a cerebral spinal fluid of said subject that is about 10% or greater compared to the peak dopamine receptor agonist concentration in the blood plasma of said subject.

9. The formulation of claim 1, wherein said formulation mucosally administered to said subject yields a peak dopamine receptor agonist concentration in a cerebral spinal fluid of said subject that is about 15% or greater compared to the peak dopamine receptor agonist concentration in the blood plasma of said subject.

10. The formulation of claim 1, wherein said formulation mucosally administered to said subject yields a peak dopamine receptor agonist concentration in a cerebral spinal fluid of said subject that is about 20% or greater compared to the peak dopamine receptor agonist concentration in the blood plasma of said subject.

11. The formulation of claim 1, wherein said formulation mucosally administered to said subject yields a peak dopamine receptor agonist concentration in a cerebral spinal fluid of said subject that is about 25% or greater compared to the peak dopamine receptor agonist concentration in the blood plasma of said subject

12. The formulation of claim 1, wherein said formulation mucosally administered to said subject yields a peak dopamine receptor agonist concentration in a cerebral spinal fluid of said subject that is about 30% or greater compared to the peak dopamine receptor agonist concentration in the blood plasma of said subject.

13. The formulation of claim 1, wherein said formulation mucosally administered to said subject yields a peak dopamine receptor agonist concentration in a cerebral spinal fluid of said subject that is about 35% or greater compared to the peak dopamine receptor agonist concentration in the blood plasma of said subject.

14. The formulation of claim 1, wherein said formulation mucosally administered to said subject yields a peak dopamine receptor agonist concentration in a cerebral spinal fluid of said subject that is about 40% or greater compared to the peak dopamine receptor agonist concentration in the blood plasma of said subject.

15. A stable pharmaceutical formulation comprising apomorphine and one or more delivery-enhancing agents, wherein said formulation following mucosal adminstration to a mammalian subject yields a peak concentration of said apomorphine in a central nervous system tissue or fluid of said subject that is 10% or greater compared to a peak concentration of said apomorphine in a blood plasma of said subject.

16. A stable pharmaceutical formulation comprising one or more dopamine receptor agonist(s) and one or more delivery-enhancing agent(s), wherein said formulation following mucosal adminstration to a mammalian subject yields a peak concentration of said dopamine receptor agonist in a central nervous system tissue or fluid of said subject that is greater than a peak concentration of said dopamine receptor agonist in the central nervous system tissue or fluid of said subject following administration to said subject of the same concentration concentration or dose of said dopamine receptor agonist to said subject by injection.

17. The formulation of claim 16, wherein the dopamine receptor agonist is apomorphine or a pharmaceutically acceptable salt or derivative thereof.

18. The formulation of claim 16, wherein said delivery-enhancing agent(s) is/are selected from:
(a) an aggregation inhibitory agent;
(b) a charge modifying agent;
(c) a pH control agent;
(d) a degradative enzyme inhibitory agent;
(e) a mucolytic or mucus clearing agent;
(f) a ciliostatic agent;
(g) a membrane penetration-enhancing agent selected from (i) a surfactant, (ii) a bile salt, (ii) a phospholipid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a clyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, or (x) an inhibitor of cholesterol synthesis; or (xi) any combination of the membrane penetration enhancing agents recited in (i)-(x);
(h) a modulatory agent of epithelial junction physiology;
(i) a vasodilator agent;
(j) a selective transport-enhancing agent; and
(k) a stabilizing delivery vehicle, carrier, support or complex-forming species with which the dopamine receptor agonist is effectively combined, associated, contained, encapsulated or bound resulting in stabilization of the dopamine receptor agonist for enhanced mucosal delivery, wherein the formulation of said dopamine receptor agonist with said one or more delivery-enhancing agents provides for increased bioavailability of the dopamine receptor agonist in a central nervous system tissue or fluid of said subject.

19. The formulation of claim 16, wherein said delivery-enhancing agent(s) is/are selected from the group consisting of citric acid, sodium citrate, propylene glycol, glycerin, L-ascorbic acid, sodium metabisulfite, edetate disodium, benzalkonium chloride, sodium hydroxide and mixtures thereof.

20. The formulation of claim 1 or 16, wherein said formulations are substantially particulate free.

21. A method for treating or preventing a disease or condition in a mammalian subject amenable to treatment by therapeutic administration of a dopamine receptor agonist, comprising mucosally administering to said subject a pharmaceutical formulation comprising a dopamine receptor agonist and one or more delivery-enhancing agent(s) resulting in delivery of a peak concentration of said dopamine receptor agonist in a central nervous system tissue or fluid of said subject that is 5% or greater compared to a peak concentration of said dopamine receptor agonist in a blood plasma of said subject.

22. The method of claim 21, wherein said disease or condition amenable to treatment by therapeutic administration of said dopamine receptor agonist is Parkinson's disease.

23. The method of claim 21, wherein said disease or condition amenable to treatment by therapeutic administration of said dopamine receptor agonist is male or female erectile dysfunstion.

24. The method of claim 21, wherein said disease or condition amenable to treatment by therapeutic administration of said dopamine receptor agonist is sexual dysfunction.

25. The method of claim 21, wherein said disease or condition amenable to treatment by therapeutic administration of said dopamine receptor agonist is male or female erectile dysfunction marked by engorgement of a male or female erectile tissue erectile tissue and/or enhanced neural stimulation potential of said erectile tissue, diminished sexual desire, or a diminished ability to reach orgasm during sexual stimulation in a male or female mammalian subject.

26. The method of claim 21, wherein said mucosal administration involves delivery of said formulation to a nasal mucosal surface of said subject.

27. The method of claim 21, wherein the dopamine receptor agonist is apomorphine or a pharmaceutically acceptable salt or derivative thereof.

28. The method of claim 21, wherein said dopamine receptor agonist is administered to said subject in an effective dose of between about 0.25 and 2.0 mg.

29. The method of claim 21, wherein said delivery-enhancing agent(s) is/are selected from:
(a) an aggregation inhibitory agent;
(b) a charge modifying agent;
(c) a pH control agent;
(d) a degradative enzyme inhibitory agent;
(e) a mucolytic or mucus clearing agent;
(f) a ciliostatic agent;
(g) a membrane penetration-enhancing agent selected from (i) a surfactant, (ii) a bile salt, (ii) a phospholipid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a clyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, or (x) an inhibitor of cholesterol synthesis; or (xi) any combination of the membrane penetration enhancing agents recited in (i)-(x);
(h) a modulatory agent of epithelial junction physiology;
(i) a vasodilator agent;
(j) a selective transport-enhancing agent; and
(k) a stabilizing delivery vehicle, carrier, support or complex-forming species with which the dopamine receptor agonist is effectively combined, associated, contained, encapsulated or bound resulting in stabilization of the dopamine receptor agonist for enhanced mucosal delivery, wherein the formulation of said dopamine receptor agonist with said one or more delivery-enhancing agents provides for increased bioavailability of the dopamine receptor agonist in a central nervous system tissue or fluid of said subject.

30. The method of claim 21, wherein said delivery-enhancing agent(s) is/are selected from the group consisting of citric acid, sodium citrate, propylene glycol, glycerin, L-ascorbic acid, sodium metabisulfite, edetate disodium, benzalkonium chloride, sodium hydroxide and mixtures thereof.

31. The method of claim 21, wherein said formulation mucosally administered to said subject yields a peak dopamine receptor agonist concentration in a cerebral spinal fluid of said subject that is between about 5-10% of the peak dopamine receptor agonist concentration in the blood plasma of said subject.

32. The method of claim 21, wherein said formulation mucosally administered to said subject yields a peak dopamine receptor agonist concentration in a cerebral spinal fluid of said subject that is about 10% or greater compared to the peak dopamine receptor agonist concentration in the blood plasma of said subject.

33. The method of claim 21, wherein said formulation mucosally administered to said subject yields a peak dopamine receptor agonist concentration in a cerebral spinal fluid of said subject that is about 15% or greater compared to the peak dopamine receptor agonist concentration in the blood plasma of said subject.

34. The method of claim 21, wherein said formulation mucosally administered to said subject yields a peak dopamine receptor agonist concentration in a cerebral spinal fluid of said subject that is about 20% or greater compared to the peak dopamine receptor agonist concentration in the blood plasma of said subject.

35. The method of claim 21, wherein said formulation mucosally administered to said subject yields a peak dopamine receptor agonist concentration in a cerebral spinal fluid of said subject that is about 25% or greater compared to the peak dopamine receptor agonist concentration in the blood plasma of said subject

36. The method of claim 21, wherein said formulation mucosally administered to said subject yields a peak dopamine receptor agonist concentration in a cerebral spinal fluid of said subject that is about 30% or greater compared to the peak dopamine receptor agonist concentration in the blood plasma of said subject.

37. The method of claim 21, wherein said formulation mucosally administered to said subject yields a peak dopamine receptor agonist concentration in a cerebral spinal fluid of said subject that is about 35% or greater compared to the peak dopamine receptor agonist concentration in the blood plasma of said subject.

38. The method of claim 21, wherein said formulation mucosally administered to said subject yields a peak dopamine receptor agonist concentration in a cerebral spinal fluid of said subject that is about 40% or greater compared to the peak dopamine receptor agonist concentration in the blood plasma of said subject.

39. The method of claim 21, which yields a peak concentration of said dopamine receptor agonist in a central nervous system tissue or fluid of said subject that is 10% or greater compared to a peak concentration of said apomorphine in a blood plasma of said subject.

40. The method of claim 21, which yields a peak concentration of said dopamine receptor agonist in a central nervous system tissue or fluid of said subject that is greater than a peak concentration of said dopamine receptor agonist in the central nervous system tissue or fluid of said subject following administration to the subject of the same concentration or dose of said dopamine receptor agonist by injection.

41. The method of claim 21, wherein the dopamine receptor agonist is apomorphine or a pharmaceutically acceptable salt or derivative thereof.

42. The method of claim 21, wherein said delivery-enhancing agent(s) is/are selected from:
(a) an aggregation inhibitory agent;
(b) a charge modifying agent;
(c) a pH control agent;
(d) a degradative enzyme inhibitory agent;
(e) a mucolytic or mucus clearing agent;
(f) a ciliostatic agent;
(g) a membrane penetration-enhancing agent selected from (i) a surfactant, (ii) a bile salt, (ii) a phospholipid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a clyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, or (x) an inhibitor of cholesterol synthesis; or (xi) any combination of the membrane penetration enhancing agents recited in (i)-(x);
(h) a modulatory agent of epithelial junction physiology;
(i) a vasodilator agent;
(j) a selective transport-enhancing agent; and
(k) a stabilizing delivery vehicle, carrier, support or complex-forming species with which the dopamine receptor agonist is effectively combined, associated, contained, encapsulated or bound resulting in stabilization of the dopamine receptor agonist for enhanced mucosal delivery, wherein the formulation of said dopamine receptor agonist with said one or more delivery-enhancing agents provides for increased bioavailability of the dopamine receptor agonist in a central nervous system tissue or fluid of said subject.

43. The method of claim 21, wherein said delivery-enhancing agent(s) is/are selected from the group consisting of citric acid, sodium citrate, propylene glycol, glycerin, L-ascorbic acid, sodium metabisulfite, edetate disodium, benzalkonium chloride, sodium hydroxide and mixtures thereof.

44. The formulation of claim 1, which is substantially particulate free.

45. A method for treating or preventing a disease or condition in a mammalian subject amenable to treatment by therapeutic administration of a dopamine receptor agonist, comprising coordinately, mucosally administering to said subject a dopamine receptor agonist and one or more delivery-enhancing agent(s) resulting in delivery of a peak concentration of said dopamine receptor agonist in a central nervous system tissue or fluid of said subject that is 5% or greater compared to a peak concentration of said dopamine receptor agonist in a blood plasma of said subject.

46. The method of claim 45, wherein said dopamine receptor agonist and said delivery enhancing agent are each administered to a mucosal tissue of said subject.

47. The method of claim 45, wherein delivery enhancing agent is administered to said subject by a different route of delivery than mucosal administration.

48. The method of claim 45, wherein said dopamine receptor agonist and said delivery enhancing agent are each administered simultaneously to a mucosal tissue of said subject.

49. The method of claim 45, wherein said dopamine receptor agonist and said delivery enhancing agent are administered to said subject together in a combinatorial formulation.

50. A method for preparing a phamaceutical formulation of a dopamine receptor agonist for mucosal administration and enhanced delivery to a central nervous system fluid or tissue of a mammalian subject comprising preparing a combinatorial formulation of at least one dopamine receptor agonist and at least one delivery-enhancing agent selected from:
(a) an aggregation inhibitory agent;
(b) a charge modifying agent;
(c) a pH control agent;
(d) a degradative enzyme inhibitory agent;
(e) a mucolytic or mucus clearing agent;
(f) a ciliostatic agent;
(g) a membrane penetration-enhancing agent selected from (i) a surfactant, (ii) a bile salt, (ii) a phospholipid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a clyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, or (x) an inhibitor of cholesterol synthesis; or (xi) any combination of the membrane penetration enhancing agents recited in (i)-(x);
(h) a modulatory agent of epithelial junction physiology;
(i) a vasodilator agent;
(j) a selective transport-enhancing agent; and
(k) a stabilizing delivery vehicle, carrier, support or complex-forming species with which the dopamine receptor agonist is effectively combined, associated, contained, encapsulated or bound resulting in stabilization of the active agent for enhanced intranasal delivery, wherein the formulation of said dopamine receptor agonist with said delivery-enhancing agent(s) provides for increased bioavailability of the dopamine receptor agonist in a central nervous system tissue or fluid of said subject.

51. The pharmaceutcal formulation of claim 1, comprising a plurality of mucosal delivery-enhancing agents selected from:
(a) an aggregation inhibitory agent;
(b) a charge modifying agent;
(c) a pH control agent;
(d) a degradative enzyme inhibitory agent;
(e) a mucolytic or mucus clearing agent;
(f) a ciliostatic agent;
(g) a membrane penetration-enhancing agent selected from (i) a surfactant, (ii) a bile salt, (ii) a phospholipid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a clyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, or (x) an inhibitor of cholesterol synthesis; or (xi) any combination of the membrane penetration enhancing agents recited in (i)-(x);
(h) a modulatory agent of epithelial junction physiology;
(i) a vasodilator agent;
(j) a selective transport-enhancing agent; and
(k) a stabilizing delivery vehicle, carrier, support or complex-forming species with which the dopamine receptor agonist is effectively combined, associated, contained, encapsulated or bound resulting in stabilization of the dopamine receptor agonist for enhanced mucosal delivery, wherein said plurality of mucosal delivery-enhancing agents comprises any combination of two or more of said mucosal delivery-enhancing agents recited in (a)-(k), and wherein coordinate administration of said dopamine receptor agonist with said plurality of mucosal delivery-enhancing agents provides for increased bioavailability of the dopamine receptor agonist delivered to a mucosal mucosal surface of said subject.

52. The pharmaceutical formulation of claim 1, which incorporates a plurality of reducing agents to stabilize the dopamine receptor agonist.

53. The pharmaceutical formulation of claim 1, which incorporates a chitosan or chitosan derivative.

54. The pharmaceutical formulation of claim 1, wherein said chitosan or chitosan derivative is poly-GuD.

55. The pharmaceutical formulation of claim 1, which is pH adjusted to between about pH 3.0-6.0.

56. The pharmaceutical formulation of claim 1, which is pH adjusted to between about pH 3.0-5.0.

57. The pharmaceutical formulation of claim 1, which is pH adjusted to between about pH 3.0-4.0.

58. The pharmaceutical formulation of claim 1, which is pH adjusted to about pH 3.0-3.5.
